(19) United States
(12) Patent Application Publication
(10) Pub. No.: US 2026/0193278 A1
(43) Pub. Date: Jul. 9, 2026

(54) NOVEL PHTHALOCYANINE COMPOUNDS AND THE USE THEREOF FOR THE TREATMENT OF DISEASES

(71) Applicant: RHODES UNIVERSITY, Makhanda (ZA)

(72) Inventors: Tebello Nyokong, Makhanda (ZA); Pinar Sen, Istanbul (TR)

(73) Assignee: RHODES UNIVERSITY, Makhanda (ZA)

(21) Appl. No.: 19/134,436

(22) PCT Filed: Jan. 16, 2023
(86) PCT No.: PCT/IB2023/050363

§ 371 Date: May 30, 2025

(30) Foreign Application Priority Data
Dec. 2, 2022 (ZA) 2022/13068

(51) Int. Cl. C07F 3/06; A61K 31/555; A61K 41/00; A61P 31/04; C07F 1/08; C07F 15/06

(52) U.S. Cl. CPC C07F 3/06; A61K 31/555; A61K 41/0071; A61P 31/04; C07F 1/08; C07F 15/065

(57) ABSTRACT

The present invention relates to novel phthalocyanines compounds and the use thereof to treat diseases, and more particularly, to treat diseases that include bacterial infections, cancer, cardiovascular, high blood pressure, viral infections and the like. The compounds have a general formula (I) wherein M is metal selected from the group consisting of zinc, cobalt, copper and nickel; R₁-R₄ is selected from the group consisting of 4-(5-chloro-1H-benzo[d]imidazole-2-yl)phenoxy tetraiodide; 4-(4-bromo-6-fluoro-1H-benzo[d]imidazole)phenoxy tetraiodide; 4-(4,6-dichloro-1H-benzo[d]imidazole-2-yl)phenoxy tetraiodide; 4-(5-bromo-1H-benzo[d]imidazole-2-yl)phenoxy tetraiodide; 4-(5,6-dichloro-1H-benzo[d]imidazole-2-yl)phenoxy tetraiodide; 4-(6-bromo-4-chloro-1H-benzo[d]imidazole-2-yl)phenoxy tetraiodide, and combinations thereof.

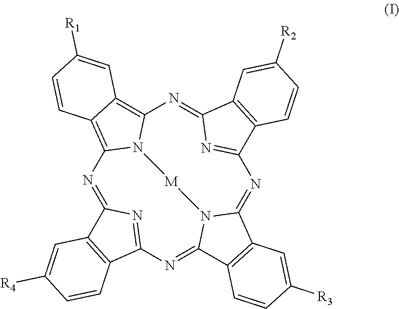

### FIELD OF APPLICATION OF THE INVENTION

The present invention relates to novel phthalocyanines compounds and the use thereof to treat diseases, and more particularly, to treat diseases that include bacterial infections, cancer, cardiovascular, high blood pressure, viral infections and the like.

### BACKGROUND TO THE INVENTION

Pathogenic microbes including bacteria, viruses, protozoans and fungi are responsible for a number of infectious diseases. Treatment of diseases caused by such pathogens, as well as the sterilization of surfaces and foods as a prophylactic measure, currently include the use of antimicrobial drugs, UV irradiation, thermotherapy and the like.

However, drug-resistant infections are a growing concern and, with specific regard to drug resistant bacterial activity. A 2019 report estimated that more than 2.8 million antibiotic-resistant infections occur in the US each year, and more than 35 000 people die as a result (https://www.cdc.gov/drugresistance/biggest-threats). As such, the treatment of microbial infections, without causing drug resistance, has become a necessity.

Furthermore, the current sterilization techniques as mentioned above require excessive amounts of energy and so are neither feasible nor are they sustainable.

Currently, a method of anti-drug-resistant antimicrobial therapy known as photodynamic therapy (PDT) has been increasingly utilized; with a closely related method known as photodynamic antimicrobial chemotherapy (PACT) being utilized to treat microbial infections. PACT utilizes a photosensitive compound (photosensitizer) which, when irradiated by incident light of a particular wavelength, gets to an excited singlet state which may subsequently undergo intersystem crossing to the triplet state. Thereafter, the triplet state photosensitizer transfers its excess energy to a ground state molecular oxygen to form a singlet oxygen, which itself is capable of inducing oxidative damage and is thus toxic when it interacts with cells or tissues of the targeted microbes and/or other target cells. The photodynamic activity damage occurs mainly in the cytoplasmic membrane and in the DNA of the target microbe, and may include leakage of cellular contents, inactivation of membrane transport systems and enzymes, and the inactivation of DNA repair mechanisms.

As such, PACT is considered a highly effective, alternative method of antibacterial therapy to the conventional use of antibiotic drugs, as it has several advantages over antibiotics, which notably includes the fact that it is not possible for the microbes to develop a resistance to the photosensitizers. In addition to the use of these compounds to kill microbial infections, these compounds are often also efficient in PDT to treat a variety of cancer types.

Phthalocyanines are multifunctional macrocycle compounds which have been shown to be efficient photosensitizers in the treatment of pathogenic microbes through the use of PACT. They can form long-lived triplet excited states, and as a result show high singlet oxygen production which in turn causes the oxidative damage on the target cells and tissues. Furthermore, it has been found that phthalocyanines carrying cationic substituents are more successful at passing through the anionic outer bacterial surface and thus have enhanced solubility and cellular uptake.

Whilst the PACT method provides an efficient means of treating microbial infections without the consequent result of antimicrobial resistance, the mechanism of action of such therapy is heavily reliant on the presence of light in order to stimulate the photosensitizer and subsequently induce the oxidative damage. As such, whilst it is clearly an effective means of surface sterilization, as well as that of topical treatment on patients, it is difficult to use this method of treatment successfully in regions of the human body which are difficult to access.

Given the above, it is clear that there exists a present need for antimicrobial compounds, the use of which does not subsequently cause antimicrobial drug resistance, which are capable of efficiently eradicating the infectious pathogens without light stimulation, thereby permitting their use in regions of the human body which are inaccessible to light. Moreover, there is a need for a compound that is capable of disinfecting surfaces and killing microbial infections without the need for high-energy ultraviolet (UV) irradiation or thermotherapy.

### OBJECT OF THE INVENTION

It is accordingly an object of the present invention to provide novel compounds and their uses that seek to, at least partially, overcome or lessen the above disadvantages and/or will be a useful alternative to the existing antimicrobial therapies and sterilization techniques.

### SUMMARY OF THE INVENTION

According to a first aspect of the present invention, there is provided a compound of general Formula (I)

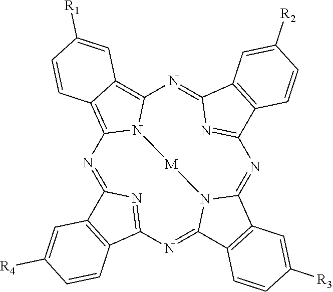

wherein
M is metal selected from the group consisting of zinc, indium, gallium cobalt, copper and nickel;
R₁-R₄ is selected from the group consisting of

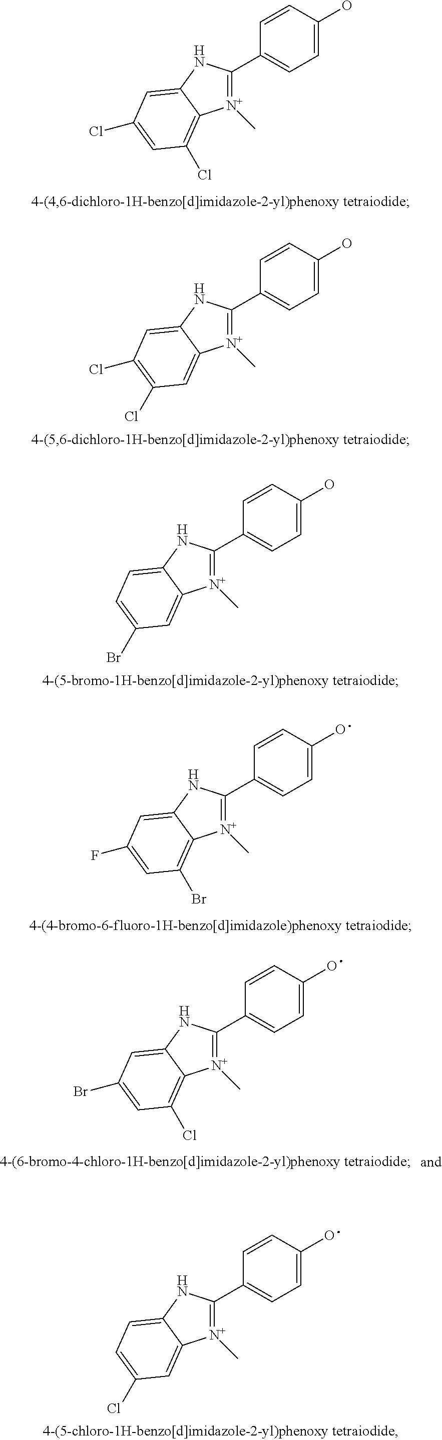

and combinations thereof.

The substituents at R₁-R₄ may be selected from the group consisting of

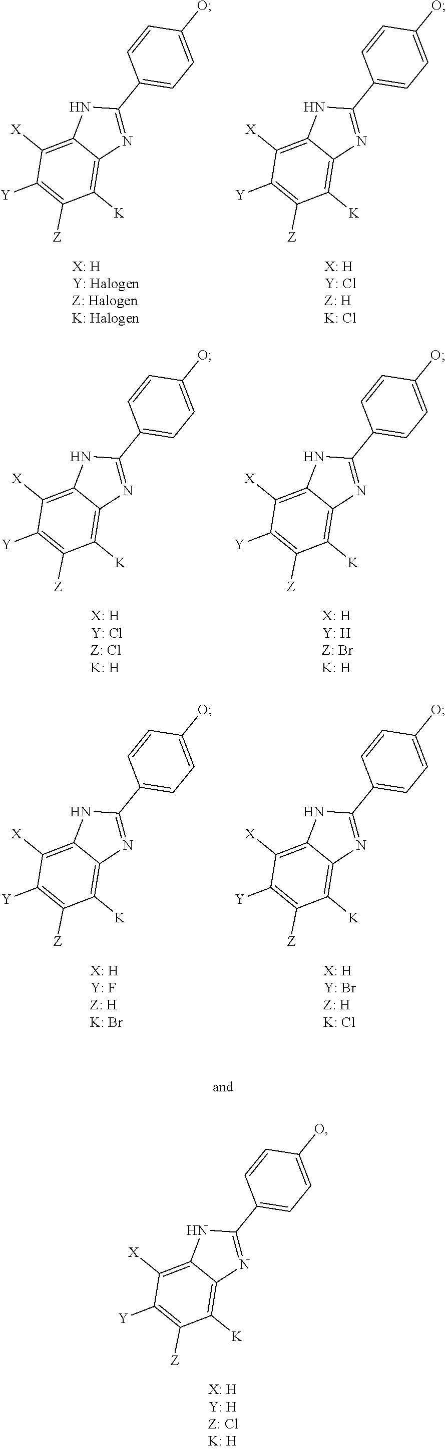

and combinations thereof.

The compound according to Formula I may be used in the treatment of microbial infections.

The compound according to Formula I may be used in the treatment of a variety of cancer and pre-cancer types.

In a further embodiment of the invention, the compound according to Formula I may be used as an antifungal treatment or as a fungicide.

In an alternative embodiment of the invention, the compound according to Formula I as described herein may be used as an antioxidant reducing the effects of reactive oxygen species on the human body.

The compound according to Formula I may be used in the treatment of diseases resulting from oxidative damage, which may be selected from the group consisting of atherosclerosis, inflammatory injury, cancer and cardiovascular disease.

The compound according to Formula I may be used as an analgesic.

There is provided for the compound according to Formula I to be used as an anti-inflammatory agent for reducing inflammation.

There is further provided for the compound according to Formula I to be used as an anti-ulcer agent.

The compound according to Formula I may be used as an antiviral for the treatment of one or more viruses.

The invention provides for the compound according to Formula I to be used as an antiprotozoal agent.

In an embodiment of the invention, the compound according to Formula I may be used in the treatment of a protozoan infection. Alternatively, the compound may be used as an antihistaminic drug. In a further embodiment of the invention, the compound may be used as an antihypertensive drug for the treatment of hypertension.

According to a second aspect of the invention, there is provided for use of the compound according to Formula I, as described herein, in the treatment of one or more conditions in a subject.

The condition may be selected from the group consisting of microbial infection, cancer, fungal infection, fungal disease, oxidative damage, oxidative-related disease, atherosclerosis, inflammatory injury, cardiovascular disease, analgesic disease, inflammation, ulcer, ulcer-related disease, viral infection, protozoal-related disease, protozoan infection, allergy, fever, hypertension, and a combination thereof.

According to a third aspect of the invention, there is provided for use of the compound according to Formula I, as described herein, in the manufacture of a medicament for the treatment of one or more conditions in a subject as set out above.

According to a fourth aspect of the invention, there is provided for a method of treating one or more conditions in a subject, as described herein, the method comprising the steps of administering to the subject a therapeutically effective amount of the compound according to Formula I as described herein.

The compounds of the invention be used as an aqueous solution, cream, gel, ointment, liposomal formulation, tablet, capsule, suspension and a combination thereof.

The invention further provides for the compound to be combined with a secondary compound selected from the group consisting of cellulose, chitosan, Cyclodextrin, Nucleic acid aptamers, silver nanoparticles, iron/silver nanoparticles, Exopolysaccharides, Alginates, Proteins, Polylactic acid, Lignin, and a combination thereof.

The combination of the compound according to Formula I with the secondary compound may be used to improve drug delivery.

### BRIEF DESCRIPTION OF THE FIGURES

The invention will now further be described, by way of example only, with reference to the accompanying Figures wherein:

FIG. 1 Graphs showing the antimicrobial and anticancer studies of compound 193;

FIG. 2 Graphs showing the antimicrobial and anticancer studies of compound 200;

FIG. 3 Graphs showing the antimicrobial and anticancer studies of compound 201;

FIG. 4(a) Graph showing the concentration studies for compounds 193, 200 and 201;

FIG. 4(b) Graph showing the concentration studies for compounds 193, 200 and 201;

FIG. 5 Agar plate micrographs (a); (b); (c) and (d) showing the visualization of bacteria cell death of compounds 193, 200 and 201;

FIG. 6(a) Graph showing the concentration studies for compounds 207, 208 and 209;

FIG. 6(b) Graph showing the concentration studies for compounds 207, 208 and 209;

FIG. 7 Agar plate micrographs (a); (b); (c) and (d) showing the visualization of bacteria cell death of compounds 207, 208 and 209;

FIG. 8(a) Graph showing the concentration studies for compounds 217, 218 and 219;

FIG. 8(b) Graph showing the concentration studies for compounds 217, 218 and 219;

FIG. 9 Agar plate micrographs (a); (b); (c) and (d) showing the visualization of bacteria cell death of compounds 217, 218 and 219;

FIG. 10(a) Graph showing the concentration studies for compounds 229, 230 and 231;

FIG. 10(b) Graph showing the concentration studies for compounds 229, 230 and 231;

FIG. 11 Agar plate micrographs (a); (b); (c) and (d) showing the visualization of bacteria cell death of compounds 229, 230 and 231;

FIG. 12(a) Graph showing the concentration studies for compounds 233, 234 and 235;

FIG. 12(b) Graph showing the concentration studies for compounds 233, 234 and 235;

FIG. 13 Agar plate micrographs (a); (b); (c) and (d) showing the visualization of bacteria cell death of compounds 233, 234 and 235;

FIG. 14(a) Graph showing the concentration studies for compounds 242, 243, 244;

FIG. 14(b) Graph showing the concentration studies for compounds 242, 243, 244;

FIG. 15 Agar plate micrographs (a); (b); (c) and (d) showing the visualization of bacteria cell death of compounds 242, 243, 244;

FIG. 16 FT-IR spectrum of compound 180;

FIG. 17 FT-IR spectrum of compound 181;

FIG. 18 FT-IR spectrum of compound 182;

FIG. 19 FT-IR spectrum of compound 182;

FIG. 20 FT-IR spectrum of compound 185;

FIG. 21 FT-IR spectrum of compound 215;

FIG. 22 FT-IR spectrum of compound 186;

FIG. 23 FT-IR spectrum of compound 187;

FIG. 24 FT-IR spectrum of compound 188;

FIG. 25 FT-IR spectrum of compound 190;

FIG. 26 FT-IR spectrum of compound 191;

FIG. 27 FT-IR spectrum of compound 216;

FIG. 28 FT-IR spectrum of compound 189;

FIG. 29 FT-IR spectrum of compound 203;

FIG. 30 FT-IR spectrum of compound 211;

FIG. 31 FT-IR spectrum of compound 221;

FIG. 32 FT-IR spectrum of compound 225;

FIG. 33 FT-IR spectrum of compound 237;

FIG. 34 FT-IR spectrum of compound 197;

FIG. 35 FT-IR spectrum of compound 204;

FIG. 36 FT-IR spectrum of compound 212;

FIG. 37 FT-IR spectrum of compound 222;

FIG. 38 FT-IR spectrum of compound 226;

FIG. 39 FT-IR spectrum of compound 238;

FIG. 40 FT-IR spectrum of compound 198;

FIG. 41 FT-IR spectrum of compound 205;

FIG. 42 FT-IR spectrum of compound 213;

FIG. 43 FT-IR spectrum of compound 223;

FIG. 44 FT-IR spectrum of compound 227;

FIG. 45 FT-IR spectrum of compound 239;

FIG. 46 FT-IR spectrum of compound 193;

FIG. 47 FT-IR spectrum of compound 207;

FIG. 48 FT-IR spectrum of compound 217;

FIG. 49 FT-IR spectrum of compound 229;

FIG. 50 FT-IR spectrum of compound 233;

FIG. 51 FT-IR spectrum of compound 242;

FIG. 52 FT-IR spectrum of compound 200;

FIG. 53 FT-IR spectrum of compound 208;

FIG. 54 FT-IR spectrum of compound 218;

FIG. 55 FT-IR spectrum of compound 230;

FIG. 56 FT-IR spectrum of compound 243;

FIG. 57 FT-IR spectrum of compound 201;

FIG. 58 FT-IR spectrum of compound 209;

FIG. 59 FT-IR spectrum of compound 219;

FIG. 60 FT-IR spectrum of compound 231;

FIG. 61 FT-IR spectrum of compound 235;

FIG. 62 FT-IR spectrum of compound 244;

FIG. 63 MALDI-TOF MS spectrum of compound 180;

FIG. 64 MALDI-TOF MS spectrum of compound 181;

FIG. 65 MALDI-TOF MS spectrum of compound 182;

FIG. 66 MALDI-TOF MS spectrum of compound 184;

FIG. 67 MALDI-TOF MS spectrum of compound 185;

FIG. 68 MALDI-TOF MS spectrum of compound 215;

FIG. 69 MALDI-TOF MS spectrum of compound 186;

FIG. 70 MALDI-TOF MS spectrum of compound 187;

FIG. 71 MALDI-TOF MS spectrum of compound 188;

FIG. 72 MALDI-TOF MS spectrum of compound 190;

FIG. 73 MALDI-TOF MS spectrum of compound 191;

FIG. 74 MALDI-TOF MS spectrum of compound 216;

FIG. 75 MALDI-TOF MS spectrum of compound 189;

FIG. 76 MALDI-TOF MS spectrum of compound 203;

FIG. 77 MALDI-TOF MS spectrum of compound 211;

FIG. 78 MALDI-TOF MS spectrum of compound 221;

FIG. 79 MALDI-TOF MS spectrum of compound 225;

FIG. 80 MALDI-TOF MS spectrum of compound 237;

FIG. 81 MALDI-TOF MS spectrum of compound 197;

FIG. 82 MALDI-TOF MS spectrum of compound 204;

FIG. 83 MALDI-TOF MS spectrum of compound 212;

FIG. 84 MALDI-TOF MS spectrum of compound 222;

FIG. 85 MALDI-TOF MS spectrum of compound 226;

FIG. 86 MALDI-TOF MS spectrum of compound 238;

FIG. 87 MALDI-TOF MS spectrum of compound 198;

FIG. 88 MALDI-TOF MS spectrum of compound 205;

FIG. 89 MALDI-TOF MS spectrum of compound 213;

FIG. 90 MALDI-TOF MS spectrum of compound 223;

FIG. 91 MALDI-TOF MS spectrum of compound 227;

FIG. 92 MALDI-TOF MS spectrum of compound 239;

FIG. 93 MALDI-TOF MS spectrum of compound 193;

FIG. 94 MALDI-TOF MS spectrum of compound 207;

FIG. 95 MALDI-TOF MS spectrum of compound 217;

FIG. 96 MALDI-TOF MS spectrum of compound 229;

FIG. 97 MALDI-TOF MS spectrum of compound 233;

FIG. 98 MALDI-TOF MS spectrum of compound 242;

FIG. 99 MALDI-TOF MS spectrum of compound 200;

FIG. 100 MALDI-TOF MS spectrum of compound 208;

FIG. 101 MALDI-TOF MS spectrum of compound 218;

FIG. 102 MALDI-TOF MS spectrum of compound 230;

FIG. 103 MALDI-TOF MS spectrum of compound 234;

FIG. 104 MALDI-TOF MS spectrum of compound 243;

FIG. 105 MALDI-TOF MS spectrum of compound 201;

FIG. 106 MALDI-TOF MS spectrum of compound 209;

FIG. 107 MALDI-TOF MS spectrum of compound 219;

FIG. 108 MALDI-TOF MS spectrum of compound 231;

FIG. 109 MALDI-TOF MS spectrum of compound 235;

FIG. 110 MALDI-TOF MS spectrum of compound 244;

FIG. 111 Normalized visible absorption spectra for compounds 189, 197, 198, 193, 200, 201 in DMSO;

FIG. 112 Normalized visible absorption spectra for compounds 203, 204, 205, 207, 208, 209 in DMSO;

FIG. 113 Normalized visible absorption spectra for compounds 211, 212, 213, 217, 218, 219 in DMSO;

FIG. 114 Normalized visible absorption spectra for compounds 221, 222, 223, 229, 230, 231 in DMSO;

FIG. 115 Normalized visible absorption spectra for compounds 237, 238, 239, 242, 243, 244 in DMSO;

FIG. 116 ¹H-NMR spectrum of compound 180;

FIG. 117 ¹H-NMR spectrum of compound 186;

FIG. 118 ¹H-NMR spectrum of compound 181;

FIG. 119 ¹H-NMR spectrum of compound 187;

FIG. 120 ¹H-NMR spectrum of compound 182;

FIG. 121 ¹H-NMR spectrum of compound 188;

FIG. 122 ¹H-NMR spectrum of compound 184;

FIG. 123 ¹H-NMR spectrum of compound 190;

FIG. 124 ¹H-NMR spectrum of compound 185; and

FIG. 125 ¹H-NMR spectrum of compound 191.

### DETAILED DESCRIPTION OF THE INVENTION

The presently disclosed subject matter will now be described more fully hereinafter, in which representative embodiments are discussed. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

### Experimental

3,5-Dichloro-1,2-diaminobenzene, 4,5-Dichloro-1,2-diaminobenzene, 4-Bromo-1,2-diaminobenzene, 3-Bromo-1,2-diamino-5-fluorobenzene, 1,2-Diamino-5-bromo-3-chlorobenzene, 4-Chloro-1,2-diaminobenzene, Na₂S₂O₅, 4-nitrophthalonitrile, 4-Hydroxybenzaldehyde, methyl iodide (CH₃I), dimethylformamide (DMF), K₂CO₃, ethanol (EtOH), chloroform (CHCl₃), acetone, anhydrous ZnCl₂, Co(OAc)₂ and CuCl₂, 1,8-diazabicyclo [5.4.0]undec-7-ene (DBU), 2-Dimethylaminoethanol (DMAE).

Phosphate-buffered saline (PBS) solution pH 7.4 was prepared using appropriate amounts of Na₂HPO₄ and KH₂PO₄ in ultra-pure water from a Millipore water was from ELGA, Veolia water PURELAB, flex system (Marlow, UK). Nutrient agar and agar bacteriological BBL Muller Hinton broth were purchased from Merck. E. coli (ATCC 25922) was obtained from Microbiologic and S. aureus (ATCC 25923) was obtained Davies Diagnostics.

Cultures of MCF-7 cell were obtained from Cellonex®. Heat-inactivated fetal calf serum (FCS) and 100 unit·mL⁻¹ penicillin-100 mg·mL⁻¹ streptomycinamphotericin B were obtained from Biowest®. Dulbecco's phosphate-buffered saline (DPBS) and Dulbecco's modified Eagle's medium (DMEM) were obtained from Lonza®. Cell proliferation neutral red reagent (WST-1 assay) was obtained from Sigma-Aldrich.

### Series 1: Synthesis of Compounds 193, 200 and 201

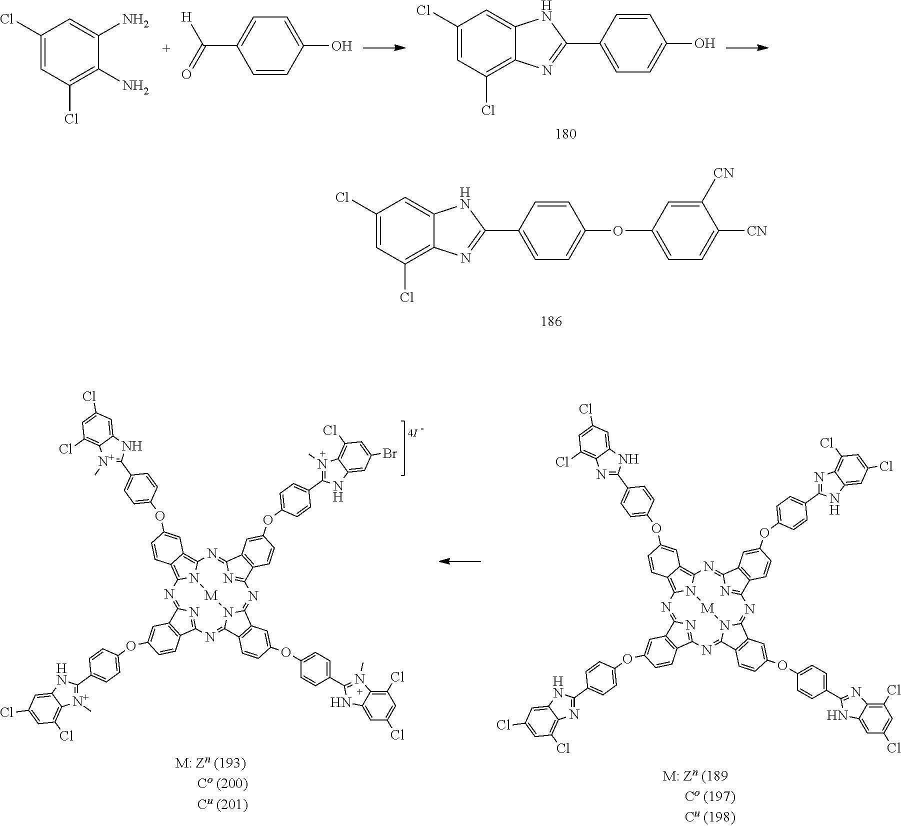

### Series 2: Synthesis of Compounds 207, 208 and 209

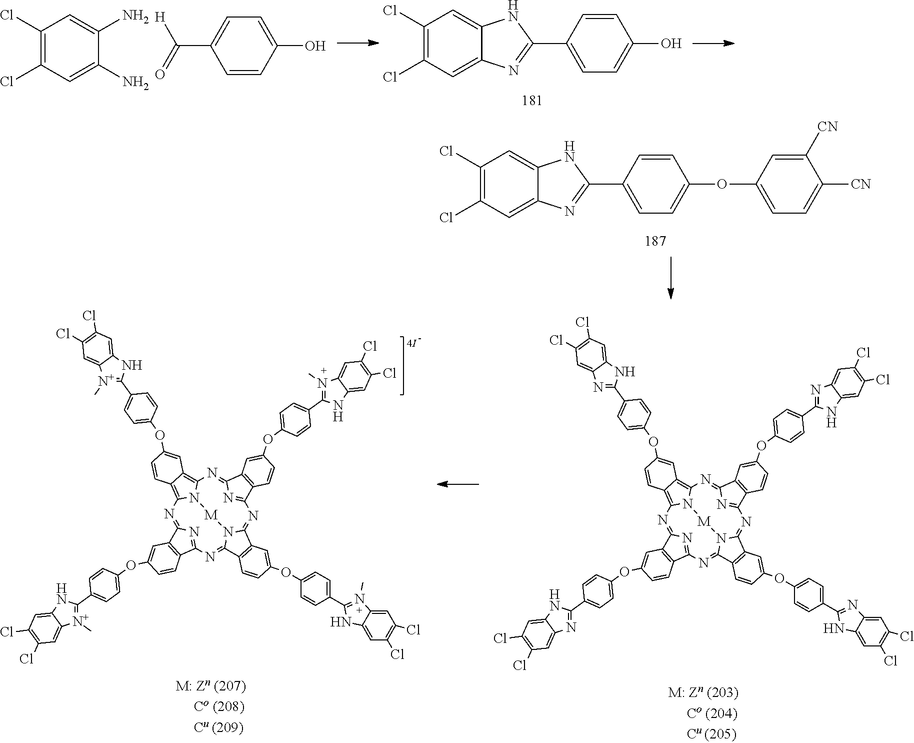

### Series 3: Synthesis of Compounds 217, 218 and 218

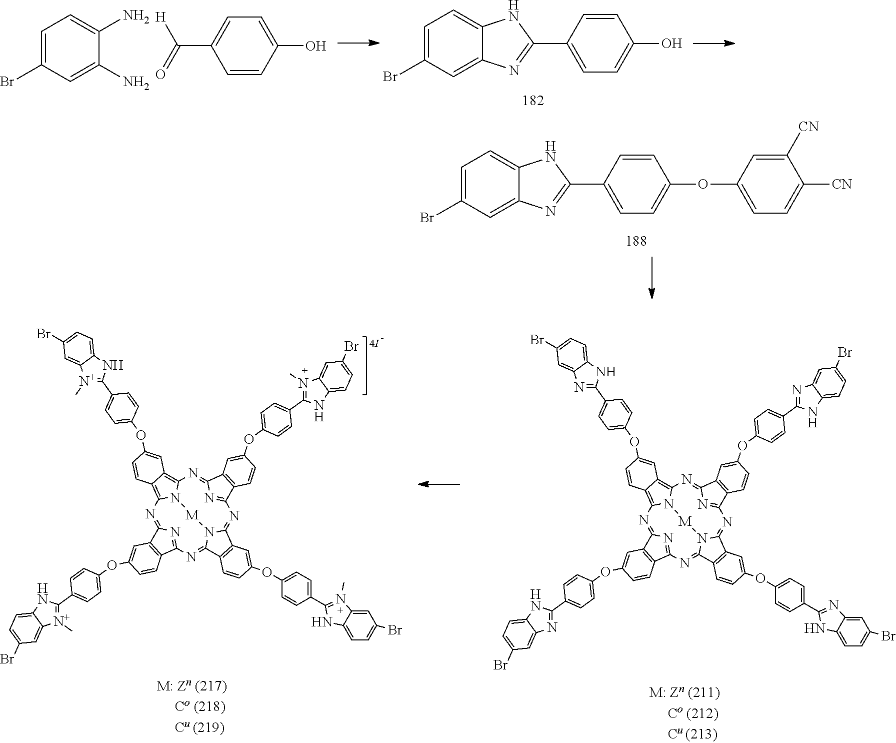

### Series 4: Synthesis of Compounds 229, 230 and 231

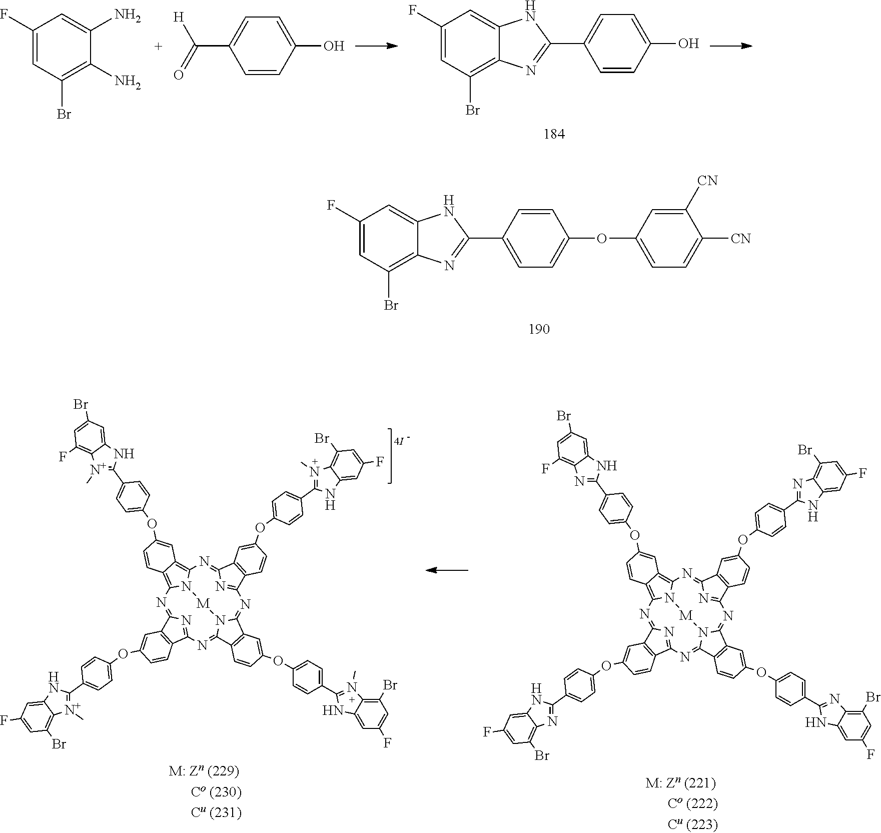

### Series 5: Synthesis of Compounds 233, 234 and 235

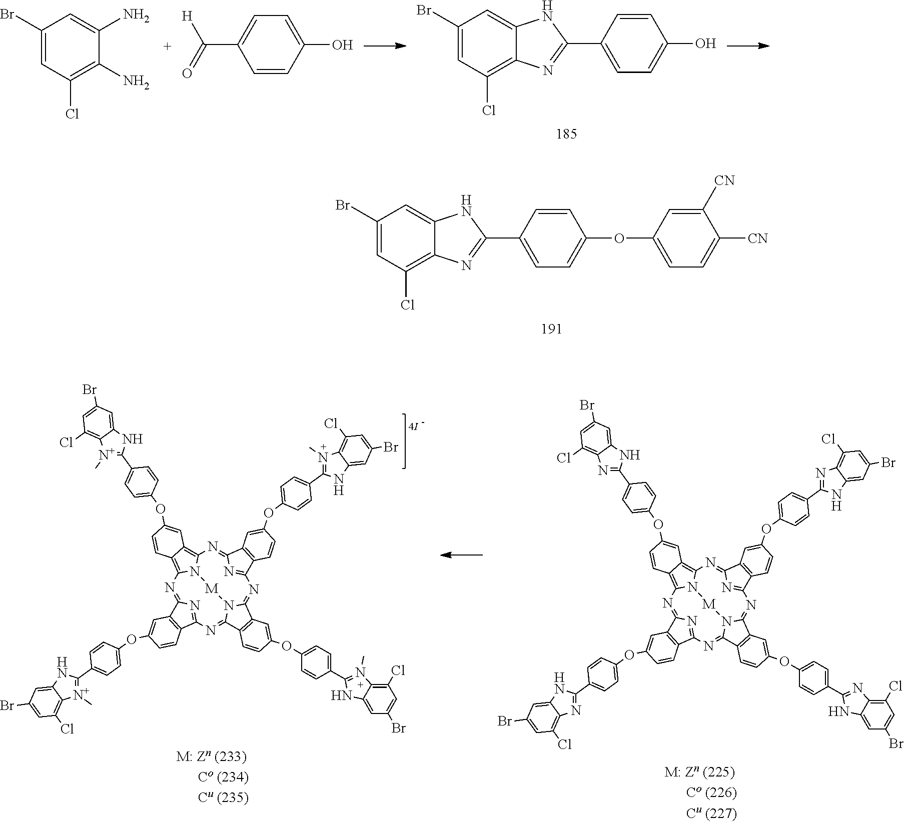

### Series 6: Synthesis of Compounds 242, 243 and 244

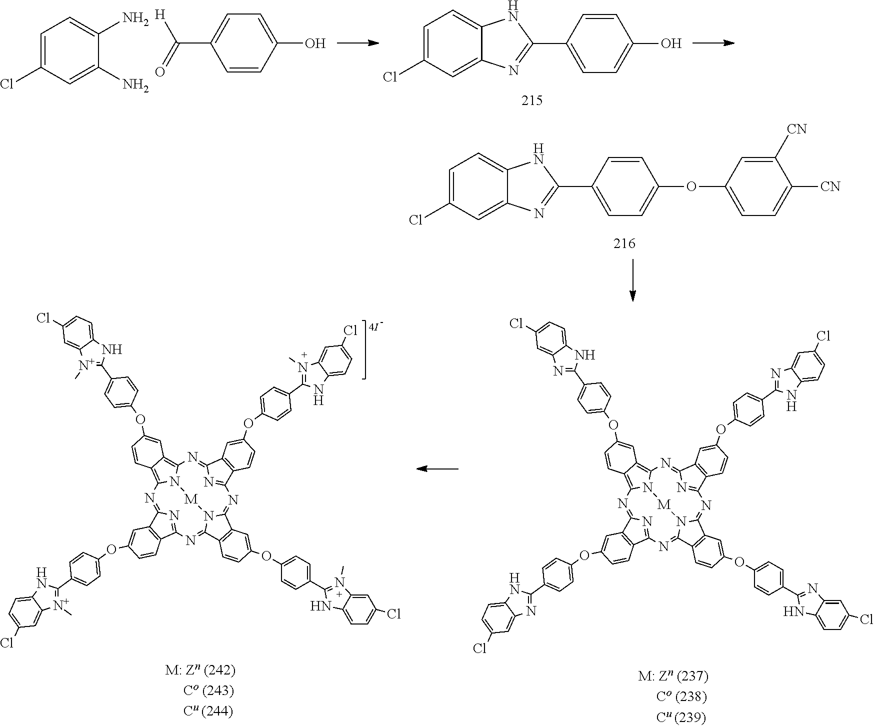

Synthesis of compounds 180, 181, 182, 184, 185 and 215

4-(4,6-dichloro-1H-benzo[d]imidazol-2-yl)phenol (180), 4-(5,6-dichloro-1H-benzo[d]imidazol-2-yl)phenol (181), 4-(5-bromo-1H-benzo[d]imidazol-2-yl)phenol (182), 4-(4-bromo-6-fluoro-1H-benzo[d]imidazol-2-yl)phenol (184), 4-(6-bromo-4-chloro-1H-benzo[d]imidazol-2-yl)phenol (185), 4-(5-chloro-1H-benzo[d]imidazol-2-yl)phenol (215).

1 eq. of the each of the 3,5-Dichloro-1,2-diaminobenzene, 4,5-Dichloro-1,2-diaminobenzene, 4-Bromo-1,2-diaminobenzene, 3-Bromo-1,2-diamino-5-fluorobenzene, 1,2-Diamino-5-bromo-3-chlorobenzene, 4-Chloro-1,2-diaminobenzene and 1 eq. of the 4-hydroxybenzaldehyde in DMF (100 ml) was treated with 1 eq. of Na₂S₂O₅. The reaction mixture heated to 90° C. for 2 h. The reaction mixture was cooled to room temperature and poured into ice/water mixture. The resulting suspension was filtered, washed with cold water and the solid was dried under vacuum.

### Characterization of Compounds 180, 181, 182, 184, 185 and 215

### Compound 180

FT-IR (UATR-TWO™) ν max/cm⁻¹: 3582 (—OH), 3292 (NH), 3088 (Ar., —CH), 1620 (C═N), 1590 (Ar., C—C), 1450-1347 (C—C), 1248 (Asym., C—O), 1178 (C—N), 1077 (Sym. C—O). ¹H-NMR (DMSO) δ ppm: 10.28 (s, 1H), 8.02 (s, 2H), 7.55 (s, 1H), 7.33 (s, 1H), 8.94 (d, 2H) 5.26 (s, 1H). ¹³C-NMR (DMSO) δ ppm: 160.14, 154.09, 138.16, 137.27, 130.49, 129.50, 127.15, 123.90, 121.21, 119.38, 119.03, 116.38, 112.17. MS (MALDI-TOF): m/z 279.46 [M]⁺, 280.39 [M+H]⁺, 303.71 [M+Na]⁺

### Compound 181

FT-IR (UATR-TWO™) ν max/cm⁻¹: 3104-2579 (NH, Ar. CH, intermolecular H bonds), 1620 (C═N), 1593 (Ar., C—C), 1447-1382 (C—C), 1244 (Asym., C—O), 1173 (C—N), 1091 (Sym. C—O). ¹H-NMR (DMSO) δ ppm: 10.30 (s, 1H), 8.02 (s, 2H), 7.87 (s, 2H), 6.98 (d, 2H), 3.82 (s, 1H). ¹³C-NMR (DMSO) δ ppm: 161.33, 153.87, 136.85, 129.72, 125.91, 117.97, 116.57, 116.06. MS (MALDI-TOF): m/z 278.56 [M]⁺, 303.90 [M+Na]⁺, 338.91 [M+Na+2H₂O]⁺.

### Compound 182

FT-IR (UATR-TWO™) ν max/cm⁻¹: 3202-2549 (NH, Ar. CH, intermolecular H bonds), 1665 (C═N), 1598 (Ar., C—C), 1455-1373 (C—C), 1242 (Asym., C—O), 1175 (C—N), 1042 (Sym. C—O). ¹H-NMR (DMSO) δ ppm: 10.15 (s, 1H), 7.98 (d, 2H), 7.71 (s, 1H), 7.49 (d, 1H), 7.30 (dd, 1H), 6.92 (d, 2H). ¹³C-NMR (DMSO) δ ppm: 162.77, 159.95, 153.49, 128.85, 124.89, 121.00, 116.23, 114.21. MS (MALDI-TOF): m/z 288.47 [M+H]⁺

### Compound 184

FT-IR (UATR-TWO™) ν max/cm⁻¹:3631 (—OH), 3388 (NH), 3082 (Ar., —CH), 1624 (C═N), 1597 (Ar., C—C), 1475-1373 (C—C), 1242 (Asym., C—O), 1178 (C—N), 1128 (Sym. C—O). ¹H-NMR (DMSO) δ ppm: 13.14 (s, 1H), 10.17 (s, 1H), 7.98 (s, 1H), 7.97 (s, 1H), 7.32 (d, 2H), 6.92 (d, 2H). ¹³C-NMR (DMSO) δ ppm: 160.04, 159.22, 157.63, 153.87, 139.79, 135.51, 128.87, 120.69, 116.28, 113.17, 111.40. MS (MALDI-TOF): m/z 306.51 [M+H]⁺

### Compound 185

FT-IR (UATR-TWO™) ν max/cm⁻¹: 3586 (—OH), 3557 (NH), 3032 (Ar., —CH), 1618 (C═N), 1593, 1573 (Ar., C—C), 1442-1362 (C—C), 1249 (Asym., C—O), 1168 (C—N), 1043 (Sym. C—O). ¹H-NMR (DMSO) δ ppm: 13.11 (s, 1H), 10.04 (s, 1H), 8.02 (d, 2H), 7.61 (s, 1H), 7.42 (s, 1H), 6.93 (d, 2H). ¹³C-NMR (DMSO) δ ppm: 160.27, 154.05, 140.87, 137.46, 129.14, 124.11, 120.46, 116.27, 113.86, 113.34, 113.32. MS (MALDI-TOF): m/z 324.50 [M+H]⁺, 346.51 [M+Na]⁺

### Compound 215

FT-IR (UATR-TWO™) ν max/cm⁻¹: 3663 (—OH), 3213 (NH), 2973 (Ar., —CH), 1609 (C═N), 1597 (Ar., C—C), 1465, 1392 (C—C), 1246 (Asym., C—O), 1178 (C—N), 1052 (Sym. C—O).

¹H-NMR (DMSO) δ ppm: 10.75 (s, 1H), 10.11 (s, 1H), 8.12 (d, 2H), 7.65 (s, 1H), 7.47 (s, 1H), 6.99 (d, 2H). ¹³C-NMR (DMSO) δ ppm: 162.89, 158.42, 154.42, 130.82, 130.74, 127.68, 124.00, 116.23, 114.21, 113.46. MS (MALDI-TOF): m/z 244.56 [M]⁺

### Synthesis of Compounds 186, 187, 188, 190, 191 and 216

4-(4-(4,6-dichloro-1H-benzo[d]imidazol-2-yl)phenoxy)phthalonitrile (186), 4-(4-(5,6-dichloro-1H-benzo[d]imidazol-2-yl)phenoxy)phthalonitrile (187), 4-(4-(5-bromo-1H-benzo[d]imidazol-2-yl)phenoxy)phthalonitrile (188), 4-(4-(4-bromo-6-fluoro-1H-benzo[d]imidazol-2-yl)phenoxy)phthalonitrile (190), 4-(4-(6-bromo-4-chloro-1H-benzo[d]imidazol-2-yl)phenoxy)phthalonitrile (191), 4-(4-(5-chloro-1H-benzo[d]imidazol-2-yl)phenoxy)phthalonitrile (216).

1 eq. of each 4-(4,6-dichloro-1H-benzo[d]imidazol-2-yl)phenol (180), 4-(5,6-dichloro-1H-benzo[d]imidazol-2-yl)phenol (181), 4-(5-bromo-1H-benzo[d]imidazol-2-yl)phenol (182), 4-(4-bromo-6-fluoro-1H-benzo[d]imidazol-2-yl)phenol (184), 4-(6-bromo-4-chloro-1H-benzo[d]imidazol-2-yl)phenol (185), 4-(5-chloro-1H-benzo[d]imidazol-2-yl)phenol (215) was mixed with the 1 eq of 4-Nitrophthalonitrile in DMF (50 mL) and degassed by nitrogen. After stirring the dissolved solution for 15 min, 2 eq. of K₂CO₃ (1.29 g, 9.32 mmol) was added gradually with stirring. The resulting suspension was stirred at room temperature for 2 days under nitrogen atmosphere. The growing of the reaction was controlled with the TLC using CHCl₃/EtOH (10/1) solvent system. The completed reaction was precipitated out by pouring into ice-water mixture. The formed precipitate was filtered off and washed with hot water, ethanol, and dried.

### Characterization of Compounds 186, 187, 188, 190, 191 and 216

### Compound 186

FT-IR (UATR-TWO™) ν max/cm⁻¹: 3281 (NH), 3082 (Ar., —CH), 2234 (C≡N), 1616 (C═N), 1582 (Ar., C—C), 1447, 1442 (C—C), 1243 (Asym., C—O), 1171 (C—N), 1080 (Sym. C—O). ¹H-NMR (DMSO) δ ppm: 9.62 (s, 1H, NH), 8.31 (d, 2H), 8.14, (d, 1H), 7.92 (s, 1H), 7.58 (s, 1H), 7.52 (d, 1H), 7.36 (d, 2H), 7.34 (s, 1H). ¹³C-NMR (DMSO) δ ppm: 160.75, 156.19, 153.66, 136.85, 129.72, 127.39, 126.78, 123.93, 123.37, 121.78, 120.87, 117.28, 116.34, 115.84, 109.32. MS (MALDI-TOF): m/z 405.77 [M]⁺.

### Compound 187

FT-IR (UATR-TWO™) ν max/cm⁻¹: 3393 (NH), 3091 (Ar., —CH), 2232 (C═N), 1663 (C═N), 1585 (Ar., C—C), 1481-1379 (C—C), 1243 (Asym., C—O), 1171 (C—N), 1090 (Sym. C—O). ¹H-NMR (DMSO) δ ppm: 9.61 (s, 1H, NH), 8.26 (d, 2H), 8.14, (d, 1H), 7.91 (s, 1H), 7.84 (s, 2H), 7.52 (d, 1H), 7.37 (d, 2H). ¹³C-NMR (DMSO) δ ppm: 162.78, 160.71, 156.29, 153.54, 136.85, 129.56, 126.99, 124.98, 123.91, 123.39, 120.97, 117.23 116.34, 115.83, 109.35.MS (MALDI-TOF): m/z 406.41 [M+H]⁺.

### Compound 188

FT-IR (UATR-TWO™) ν max/cm⁻¹:3389 (NH), 3070 (Ar., —CH), 2230 (C═N), 1659 (C═N), 1593 (Ar., C—C), 1480, 1426 (C—C), 1246 (Asym., C—O), 1169 (C—N), 1085 (Sym. C—O). ¹H-NMR (DMSO) δ ppm: 9.61 (bs, 1H, NH), 8.27 (d, 2H), 8.15 (d, 1H), 7.93 (s, 1H), 7.79 (s, 1H), 7.57 (d, 1H), 7.52 (d, 1H), 7.38 (d, 2H), 7.35 (d, 2H).

¹³C-NMR (DMSO) δ ppm: 160.80, 156.08, 152.17, 136.86, 129.40, 127.37, 125.50, 123.90, 123.35, 121.01, 117.29, 116.37, 115.85, 114.80, 109.31.MS (MALDI-TOF): m/z 415.04 [M]⁺, 437.11 [M+Na-1]⁺

### Compound 190

FT-IR (UATR-TWO™) ν max/cm⁻¹: 3321 (NH), 3070 (Ar., —CH), 2229 (C═N), 1659 (C═N), 1591 (Ar., C—C), 1478, 1413 (C—C), 1241 (Asym., C—O), 1168 (C—N), 1083 (Sym. C—O). ¹H-NMR (DMSO) δ ppm: 12.93 (bs, 1H), 8.30 (d, 2H), 8.15 (d, 1H), 7.92 (s, 1H), 7.52 (d, 1H), 7.42 (d, 1H), 7.40 (s, 1H), 7.37 (d, 2H). ¹³C-NMR (DMSO) δ ppm: 160.75, 159.48, 157.89, 156.14, 152.97, 136.86, 129.58, 127.21, 123.91, 123.36, 120.91, 117.28, 116.35, 115.84, 113.67, 113.48, 109.32. MS (MALDI-TOF): m/z 432.97 [M]⁺

### Compound 191

FT-IR (UATR-TWO™) ν max/cm⁻¹:3289 (NH), 3077 (Ar., —CH), 2232 (C≡N), 1623 (C═N), 1587 (Ar., C—C), 1478, 1420 (C—C), 1240 (Asym., C—O), 1171 (C—N), 1081 (Sym. C—O). ¹H-NMR (DMSO) δ ppm: 10.01 (s, 1H), 8.31 (d, 2H), 7.93 (d, 1H), 7.72 (s, 1H), 7.53 (dd, 1H), 7.46 (s, 1H), 7.37 (2H). ¹³C-NMR (DMSO) δ ppm: 1+60.71, 156.33, 153.15, 140.99, 136.87, 135.92, 135.74, 135.24, 129.77, 127.04, 124.43, 123.97, 123.42, 120.90, 117.29, 116.34, 115.84, 114.42, 109.37.MS (MALDI-TOF): m/z 467.25 [M+H₂O]⁺

### Compound 216

FT-IR (UATR-TWO™) ν max/cm⁻¹: 3337 (NH), 3081 (Ar., —CH), 2230 (—C═N), 1663 (C═N), 1594 (C=C), 1480, 1431 (C—C), 1243 (Asym., Ar—O—Ar), 1167 (C—N), 1053 (Sym., Ar—O—Ar). ¹H-NMR (DMSO) δ (ppm): 13.14 (d. 1H), 8.27 (d, 2H), 8.11 (d, 1H), 7.88 (d, 1H), 7.68 (d, 1H), 7.55 (d, 1H), 7.51 (dd, 1H), 7.36 (d, 2H), 7.23 (m, 1H). ¹³C-NMR (DMSO) δ (ppm): 160.79, 156.05, 152.50, 136.80, 129.39, 127.43, 123.79, 123.28, 122.61, 120.98, 120.57, 118.73, 117.27, 116.31, 115.80, 113.12, 111.54, 109.30. MS (MALDI-TOF): m/z 370.91 [M]⁺.

### Synthesis of 189, 203, 211, 221, 225, 237, 197, 204, 212, 222, 226, 238, 198, 205, 213, 223, 227 and 239:

1 eq. of the each of the 4-(4-(4,6-dichloro-1H-benzo[d]imidazol-2-yl)phenoxy)phthalonitrile (186), 4-(4-(5,6-dichloro-1H-benzo[d]imidazol-2-yl)phenoxy)phthalonitrile (187), 4-(4-(5-bromo-1H-benzo[d]imidazol-2-yl)phenoxy)phthalonitrile (188), 4-(4-(4-bromo-6-fluoro-1H-benzo[d]imidazol-2-yl)phenoxy)phthalonitrile (190), 4-(4-(6-bromo-4-chloro-1H-benzo[d]imidazol-2-yl)phenoxy)phthalonitrile (191), 4-(4-(5-chloro-1H-benzo[d]imidazol-2-yl)phenoxy)phthalonitrile (216) was mixed 0.25 eq. of anhydrous ZnCl₂, Co(OAc)₂ and CuCl₂ and catalytic amount of DBU in DMAE (4 mL). The mixture was degassed by nitrogen and stirred at 140° C. for 18 h. After being brought to ambient temperature, the mixture was allowed to precipitate with the addition of water/methanol (1/1) and subsequently collected by centrifugation. The collected compound was washed several times with ethanol, acetone and water remove the soluble by-products and any un-reacted metal salt. A better quality of the Pc was supplied by dissolving in DMF and precipitating in hot acetone for 3 times. The pure ZnPcs (189, 203, 211, 221, 225, and 237), CoPcs (197, 204, 212, 222, 226, and 238) and CuPcs (198, 205, 213, 223, 227, and 239) was accomplished as a green solid.

### Characterization of 189, 203, 211, 221, 225, 237, 197, 204, 212, 222, 226, 238, 198, 205, 213, 223, 227 and 239:

### ZnPcs:

### Compound 189: Tetrakis [4-(4-(4,6-dichloro-1H-benzo[d]imidazol-2-yl)phenoxy phthalocyaninato] zinc(II)

FT-IR (UATR-TWO™) ν max/cm⁻¹: 3375 (NH), 3065 (Ar., —CH), 1618 (C═N), 1590 (Ar., C—C), 1466, 1390 (C—C), 1228 (Asym., C—O), 1169 (C—N), 1084 (Sym. C—O), 833. UV-Vis (DMSO): λmax (nm) 679, 614, 318. MS (MALDI-TOF): m/z 1688.30 [M+2]^{+ 1}H-NMR (DMSO) δ ppm:

### Compound 203: Tetrakis [4-(4-(5,6-dichloro-1H-benzo[d]imidazol-2-yl)phenoxy phthalocyaninato] zinc(II)

FT-IR (UATR-TWO™) ν max/cm⁻¹: 3377 (NH), 3060 (Ar., —CH), 1612 (C═N), 1595 (Ar., C—C), 1474, 1413 (C—C), 1235 (Asym., C—O), 1169 (C—N), 1088 (Sym. C—O), 843. UV-Vis (DMSO): λmax (nm) 679, 613, 321. MS (MALDI-TOF): m/z 1690.45 [M+4]⁺

### Compound 211: Tetrakis [4-(4-(5-bromo-1H-benzo[d]imidazol-2-yl)phenoxy phthalocyaninato] zinc(II)

FT-IR (UATR-TWO™) ν max/cm⁻¹: 3375 (NH), 3122 (Ar., —CH), 1614 (C═N), 1596 (Ar., C—C), 1456, 1396 (C—C), 1233 (Asym., C—O), 1169 (C—N), 1049 (Sym. C—O), 835. UV-Vis (DMSO): λmax (nm) 679, 612, 317. MS (MALDI-TOF): m/z 1728.30 [M+2]⁺

### Compound 221: Tetrakis [4-(4-(4-bromo-6-fluoro-1H-benzo[d]imidazol-2-yl)phenoxy phthalocyaninato] zinc(II)

FT-IR (UATR-TWO™) ν max/cm⁻¹: 3403 (NH), 3009 (Ar., —CH), 1602 (C═N), 1596 (Ar., C—C), 1468, 1399 (C—C), 1234 (Asym., C—O), 1170 (C—N), 1094 (Sym. C—O), 835. UV-Vis (DMSO): λmax (nm) 680, 614, 316. MS (MALDI-TOF): m/z 1801.21 [M+3]⁺

### Compound 225: Tetrakis [4-(4-(6-bromo-4-chloro-1H-benzo[d]imidazol-2-yl)phenoxy phthalocyaninato] zinc(II)

FT-IR (UATR-TWO™) ν max/cm⁻¹: 3413 (NH), 3077 (Ar., —CH), 1642 (C═N), 1602, 1574 (Ar., C—C), 1483-1392 (C—C), 1231 (Asym., C—O), 1168 (C—N), 1088 (Sym. C—O), 835. UV-Vis (DMSO): λmax (nm) 679, 613, 318. MS (MALDI-TOF): m/z 1868.92 [M+4]⁺

### Compound 237: Tetrakis [4-(4-(5-chloro-1H-benzo[d]imidazol-2-yl)phenoxy phthalocyaninato] zinc(II)

FT-IR (UATR-TWO™) ν max/cm⁻¹: 3403 (NH), 3061 (Ar., —CH), 1608 (C═N), 1596 (Ar., C—C), 1471-1392 (C—C), 1233 (Asym., C—O), 1168 (C—N), 1094 (Sym. C—O), 835. UV-Vis (DMSO): λmax (nm) 679, 612, 316.MS (MALDI-TOF): m/z 1550.23 [M+2]⁺. ¹H-NMR (DMSO) δ (ppm): 13.05 (s, 4H), 8.89 (d, 4H), 8.54 (d, 4H), 8.40 (d, 4H), 8.24 (d, 4H), 7.93-7.78 (m, 4H), 7.69 (d, 8H), 7.53 (d, 8H), 7.19-7.14 (m, 4H).

CoPcs:

### Compound 197: Tetrakis [4-(4-(4,6-dichloro-1H-benzo[d]imidazol-2-yl)phenoxy phthalocyaninato] cobalt(II)

FT-IR (UATR-TWO™) ν max/cm⁻¹: 3389 (NH), 3080 (Ar., —CH), 1605 (C═N), 1529 (Ar., C—C), 1469, 1399 (C—C), 1233 (Asym., C—O), 1171 (C—N), 1090 (Sym. C—O), 838. UV-Vis (DMSO): λmax (nm) 666, 607, 319. MS (MALDI-TOF): m/z 1683.32 [M+4]⁺

### Compound 204: Tetrakis [4-(4-(5,6-dichloro-1H-benzo[d]imidazol-2-yl)phenoxy phthalocyaninato] cobalt(II)

FT-IR (UATR-TWO™) νmax/cm⁻¹: 3405(NH), 3085 (Ar., —CH), 1624 (C═N), 1598, 1525 (Ar., C—C), 1472, 1408 (C—C), 1233 (Asym., C—O), 1168 (C—N), 1091 (Sym. C—O), 840.UV-Vis (DMSO): λmax (nm) 661, 601, 321. MS (MALDI-TOF): m/z 1681.59 [M+4]⁺

### Compound 212: Tetrakis [4-(4-(5-bromo-1H-benzo[d]imidazol-2-yl)phenoxy phthalocyaninato] cobalt(II)

FT-IR (UATR-TWO™) νmax/cm⁻¹: 3412 (NH), 3058 (Ar., —CH), 1608 (C═N), 1600 (Ar., C—C), 1466, 1409 (C—C), 1232 (Asym., C—O), 1167 (C—N), 1091 (Sym. C—O), 835. UV-Vis (DMSO): λmax (nm) 662, 605, 317. MS (MALDI-TOF): m/z 1723.55 [M+4]⁺

### Compound 222: Tetrakis [4-(4-(4-bromo-6-fluoro-1H-benzo[d]imidazol-2-yl)phenoxy phthalocyaninato] cobalt(II)

FT-IR (UATR-TWO™) νmax/cm⁻¹: 3393 (NH), 3010 (Ar., —CH), 1607 (C═N), 1594 (Ar., C—C), 1466, 1400 (C—C), 1234 (Asym., C—O), 1169 (C—N), 1094 (Sym. C—O), 833. UV-Vis (DMSO): λmax (nm) 662, 601, 314. MS (MALDI-TOF): m/z 1793.16 [M+2]⁺

### Compound 226: Tetrakis [4-(4-(6-bromo-4-chloro-1H-benzo[d]imidazol-2-yl)phenoxy phthalocyaninato] cobalt(II)

FT-IR (UATR-TWO™) νmax/cm⁻¹: 3198 (NH), 3125 (Ar., —CH), 1605 (C═N), 1596 (Ar., C—C), 1468, 1398 (C—C), 1234 (Asym., C—O), 1172 (C—N), 1097 (Sym. C—O), 835. UV-Vis (DMSO): λmax (nm) 667, 603, 318. MS (MALDI-TOF): m/z 1856.09 [M]⁺

### Compound 238: Tetrakis [4-(4-(5-chloro-1H-benzo[d]imidazol-2-yl)phenoxy phthalocyaninato] cobalt(II)

FT-IR (UATR-TWO™) ν max/cm⁻¹: 3403 (NH), 3061 (Ar., —CH), 1608 (C═N), 1595 (Ar., C—C), 1460-1410 (C—C), 1233 (Asym., C—O), 1168 (C—N), 1094 (Sym. C—O), 838. UV-Vis (DMSO): λmax (nm) 665, 603, 316. MS (MALDI-TOF): m/z 1544.24 [M+1]⁺

### CuPcs:

### Compound 198: Tetrakis [4-(4-(4,6-dichloro-1H-benzo[d]imidazol-2-yl)phenoxy phthalocyaninato] copper(II)

FT-IR (UATR-TWO™) ν max/cm⁻¹: 3382 (NH), 3069 (Ar., —CH), 1615 (C═N), 1599 (Ar., C—C), 1440, 1395 (C—C), 1230 (Asym., C—O), 1165 (C—N), 1087 (Sym. C—O), 831. UV-Vis (DMSO): λmax (nm) 677, 625, 319.MS (MALDI-TOF): m/z 1687.57 [M+3]⁺

### Compound 205: Tetrakis [4-(4-(5,6-dichloro-1H-benzo[d]imidazol-2-yl)phenoxy phthalocyaninato] copper(II)

FT-IR (UATR-TWO™) ν max/cm⁻¹: 3389 (NH), 3072 (Ar., —CH), 1611 (C═N), 1597 (Ar., C—C), 1468, 1410 (C—C), 1231 (Asym., C—O), 1166 (C—N), 1089 (Sym. C—O), 840. UV-Vis (DMSO): λmax (nm) 678, 617, 321.MS (MALDI-TOF): m/z 1687.93 [M+½H₂O]⁺

### Compound 213: Tetrakis [4-(4-(5-bromo-1H-benzo[d]imidazol-2-yl)phenoxy phthalocyaninato] copper(II)

FT-IR (UATR-TWO™) ν max/cm⁻¹: 3392 (NH), 3076 (Ar., —CH), 1602 (C═N), 1599 (Ar., C—C), 1463, 1408 (C—C), 1232 (Asym., C—O), 1168 (C—N), 1092 (Sym. C—O), 836. UV-Vis (DMSO): λmax (nm) 676, 612, 315.MS (MALDI-TOF): m/z 1727.55 [M+3]⁺

### Compound 223: Tetrakis [4-(4-(4-bromo-6-fluoro-1H-benzo[d]imidazol-2-yl)phenoxy phthalocyaninato] copper(II)

FT-IR (UATR-TWO™) ν max/cm⁻¹: 3420 (NH), 3067 (Ar., —CH), 1622 (C═N), 1594, 1512 (Ar., C—C), 1471, 1399 (C—C), 1236 (Asym., C—O), 1167 (C—N), 1095 (Sym. C—O), 940, 836.UV-Vis (DMSO): λmax (nm) 677, 616, 312.MS (MALDI-TOF): m/z 1797.31 [M+1]⁺

### Compound 227: Tetrakis [4-(4-(6-bromo-4-chloro-1H-benzo[d]imidazol-2-yl)phenoxy phthalocyaninato] copper(II)

FT-IR (UATR-TWO™) νmax/cm⁻¹: 3396 (NH), 3071 (Ar., —CH), 1614 (C═N), 1598 (Ar., C—C), 1470, 1387 (C—C), 1233 (Asym., C—O), 1167 (C—N), 1096 (Sym. C—O), 834. UV-Vis (DMSO): λmax (nm) 682, 631, 318. MS (MALDI-TOF): m/z 1864.16 [M+2]⁺

### Compound 239: Tetrakis [4-(4-(5-chloro-1H-benzo[d]imidazol-2-yl)phenoxy phthalocyaninato] copper(II)

FT-IR (UATR-TWO™) ν max/cm⁻¹: 3399 (NH), 3061 (Ar., —CH), 1608 (C═N), 1595 (Ar., C—C), 1460 (C—C), 1233 (Asym., C—O), 1168 (C—N), 1094 (Sym. C—O), 838. UV-Vis (DMSO): λmax (nm) 679, 616, 316. MS (MALDI-TOF): m/z 1547.28 [M+1]⁺

### Synthesis of Quaternized Phthalocyanines;

ZnPcs (189, 203, 211, 221, 225, 237), CoPcs (197, 204, 212, 222, 226, 238) and CuPcs (198, 205, 213, 223, 227, 239), separately, was dissolved in DMF and excess CH₃I was added to the mixture. After this time, the mixture was cooled to room temperature and poured into diethyl ether. The formed precipitate was collected by centrifugation. The collected crude compound was washed several times with CHCl₃ and ethanol. The resulting quaternized Pcs was dried in vacuo.

### Characterization of 193, 200, 201, 207, 208, 209, 217, 218, 219, 229, 230, 231, 233, 234, 235, 242, 243 and 244

### ZnPcs:

### Compound 193: Tetrakis [4-(4-(4,6-dichloro-1H-benzo[d]imidazol-2-yl)phenoxy phthalocyaninato] zinc(II) tetraiodide

FT-IR (UATR-TWO™) ν max/cm⁻¹: 3415 (NH), 3005 (Ar., —CH), 2944 (Aliph., C—H), 1659 (C═N), 1594 (Ar., C—C), 1471, 1395 (C—C), 1236 (Asym., C—O), 1167 (C—N), 1088 (Sym. C—O), 939, 851. UV-Vis (DMSO): λmax (nm) 677, 610, 352. MS (MALDI-TOF): m/z 435.76 [M−4I−1]⁴⁺

### Compound 207: Tetrakis [4-(4-(5,6-dichloro-1H-benzo[d]imidazol-2-yl)phenoxy phthalocyaninato] zinc(II) tetraiodide

FT-IR (UATR-TWO™) ν max/cm⁻¹: 3408 (NH), 3008 (Ar., —CH), 2977 (Aliph., C—H), 1659 (C═N), 1595 (Ar., C—C), 1457, 1396 (C—C), 1235 (Asym., C—O), 1167 (C—N), 1089 (Sym. C—O), 838. UV-Vis (DMSO): λmax (nm) 677, 610, 356. MS (MALDI-TOF): m/z 454.11 [M−4I−H₂O]⁴⁺

### Compound 217: Tetrakis [4-(4-(5-bromo-1H-benzo[d]imidazol-2-yl)phenoxy phthalocyaninato] zinc(II) tetraiodide

FT-IR (UATR-TWO™) ν max/cm⁻¹: 3361 (NH), 3009 (Ar., —CH), 2975 (Aliph., C—H), 1661 (C═N), 1594 (Ar., C—C), 1476, 1408 (C—C), 1241 (Asym., C—O), 1167 (C—N), 1051 (Sym. C—O), 850. UV-Vis (DMSO): λmax (nm) 676, 610, 355. MS (MALDI-TOF): m/z 445.00 [M−4I−1]⁴⁺, 462.09 [M−4I−1+H₂O]⁴⁺

### Compound 229: Tetrakis [4-(4-(4-bromo-6-fluoro-1H-benzo[d]imidazol-2-yl)phenoxy phthalocyaninato] zinc(II) tetraiodide

FT-IR (UATR-TWO™) ν max/cm⁻¹: 3419 (NH), 3009 (Ar., —CH), 2898 (Aliph., C—H), 1659 (C═N), 1593, 1510 (Ar., C—C), 1463, 1400 (C—C), 1234 (Asym., C—O), 1166 (C—N), 1094 (Sym. C—O), 831. UV-Vis (DMSO): λmax (nm) 672, 606, 351. MS (MALDI-TOF): m/z 463.10 [M−4I−1]⁴⁺

### Compound 233: Tetrakis [4-(4-(6-bromo-4-chloro-1H-benzo[d]imidazol-2-yl)phenoxy phthalocyaninato] zinc(II) tetraiodide

FT-IR (UATR-TWO™) ν max/cm⁻¹: 3416 (NH), 3063 (Ar., —CH), 2992 (Aliph., C—H), 1661 (C═N), 1597 (Ar., C—C), 1466, 1395 (C—C), 1236 (Asym., C—O), 1170 (C—N), 1092 (Sym. C—O), 836. UV-Vis (DMSO): λmax (nm) 677, 611, 362. MS (MALDI-TOF): m/z 479.07 [M−4I−1]4+

### Compound 242: Tetrakis [4-(4-(5-chloro-1H-benzo[d]imidazol-2-yl)phenoxy phthalocyaninato] zinc(II) tetraiodide

FT-IR (UATR-TWO™) ν max/cm⁻¹: 3424 (NH), 3015 (Ar., —CH), 2916 (Aliph., C—H), 1659 (C═N), 1598 (Ar., C—C), 1465, 1400 (C—C), 1239 (Asym., C—O), 1168 (C—N), 1091 (Sym. C—O), 864. UV-Vis (DMSO): λmax (nm) 675, 610, 350. MS (MALDI-TOF): m/z 400.67 [M−4I−1]⁴⁺

### CoPcs:

### Compound 200: Tetrakis [4-(4-(4,6-dichloro-1H-benzo[d]imidazol-2-yl)phenoxy phthalocyaninato] cobalt(II) tetraiodide

FT-IR (UATR-TWO™) ν max/cm⁻¹: 3421 (NH), 3019 (Ar., —CH), 2938 (Aliph., C—H), 1648 (C═N), 1597, 1518 (Ar., C—C), 1468, 1404 (C—C), 1238 (Asym., C—O), 1168 (C—N), 1094 (Sym. C—O), 849. UV-Vis (DMSO): λmax (nm) 666, 606, 322. MS (MALDI-TOF): m/z 435.74 [M−4I]⁴⁺

### Compound 208: Tetrakis [4-(4-(5,6-dichloro-1H-benzo[d]imidazol-2-yl)phenoxy phthalocyaninato] cobalt(II) tetraiodide

FT-IR (UATR-TWO™) ν max/cm⁻¹: 3421 (NH), 3007 (Ar., —CH), 2971 (Aliph., C—H), 1649 (C═N), 1598 (Ar., C—C), 1458, 1404 (C—C), 1237 (Asym., C—O), 1168 (C—N), 1094 (Sym. C—O), 849. UV-Vis (DMSO): λmax (nm) 659, 598, 338. MS (MALDI-TOF): m/z 452.13 [M−4I+H₂O]⁴⁺.

### Compound 218: Tetrakis [4-(4-(5-bromo-1H-benzo[d]imidazol-2-yl)phenoxy phthalocyaninato] zinc(II) tetraiodide

FT-IR (UATR-TWO™) ν max/cm⁻¹: 3419 (NH), 3006 (Ar., —CH), 2974 (Aliph., C—H), 1642 (C═N), 1597 (Ar., C—C), 1469, 1403 (C—C), 1239 (Asym., C—O), 1167 (C—N), 1093 (Sym. C—O), 850. UV-Vis (DMSO): λmax (nm) 661, 605, 345. MS (MALDI-TOF): m/z 444.62 [M−4I]⁴⁺, 462.68 [M−4I+H₂O]⁴⁺.

### Compound 230: Tetrakis [4-(4-(4-bromo-6-fluoro-1H-benzo[d]imidazol-2-yl)phenoxy phthalocyaninato] cobalt(II) tetraiodide

FT-IR (UATR-TWO™) ν max/cm⁻¹: 3417 (NH), 3013 (Ar., —CH), 2973 (Aliph., C—H), 1645 (C═N), 1601, 1598 (Ar., C—C), 1484, 1403 (C—C), 1240 (Asym., C—O), 1170 (C—N), 1084 (Sym. C—O), 846. UV-Vis (DMSO): λmax (nm) 659, 600, 351. MS (MALDI-TOF): m/z 463.10 [M−4I]⁴⁺, 480.16 [M−4I+H₂O]⁴⁺.

### Compound 234: Tetrakis [4-(4-(6-bromo-4-chloro-1H-benzo[d]imidazol-2-yl)phenoxy phthalocyaninato] cobalt(II) tetraiodide

FT-IR (UATR-TWO™) ν max/cm⁻¹: 3421 (NH), 3016 (Ar., —CH), 2985 (Aliph., C—H), 1642 (C═N), 1594, 1514 (Ar., C—C), 1461, 1404 (C—C), 1236 (Asym., C—O), 1165 (C—N), 1095 (Sym. C—O), 838. UV-Vis (DMSO): λmax (nm) 667, 606, 347. MS (MALDI-TOF): m/z 478.81 [M−4I−1]⁴⁺, 496.02 [M−4I+−1+H₂O]⁴⁺.

### Compound 243: Tetrakis [4-(4-(5-chloro-1H-benzo[d]imidazol-2-yl)phenoxy phthalocyaninato] cobalt(II) tetraiodide

FT-IR (UATR-TWO™) ν max/cm⁻¹: 3420 (NH), 3005 (Ar., —CH), 2939 (Aliph., C—H), 1638 (C═N), 1594, 1514 (Ar., C—C), 1470, 1399 (C—C), 1239 (Asym., C—O), 1167 (C—N), 1095 (Sym. C—O), 938. UV-Vis (DMSO): λmax (nm) 659, 598, 332. MS (MALDI-TOF): m/z 400.68 [M−4I]4+, 496.02 [M−4I+−1+H₂O]⁴⁺.

### CuPcs:

### Compound 201: Tetrakis [4-(4-(4,6-dichloro-1H-benzo[d]imidazol-2-yl)phenoxy phthalocyaninato] copper(II) tetraiodide

FT-IR (UATR-TWO™) ν max/cm⁻¹: 3404 (NH), 3002 (Ar., —CH), 2956 (Aliph., C—H), 1642 (C═N), 1590 (Ar., C—C), 1461, 1396 (C—C), 1229 (Asym., C—O), 1164 (C—N), 1086, (Sym. C—O), 844. UV-Vis (DMSO): λmax (nm) 675, 607, 307. MS (MALDI-TOF): m/z 435.65 [M−4I]⁴⁺

### Compound 209: Tetrakis [4-(4-(5,6-dichloro-1H-benzo[d]imidazol-2-yl)phenoxy phthalocyaninato] copper(II) tetraiodide

FT-IR (UATR-TWO™) ν max/cm⁻¹: 3392 (NH), 3005 (Ar., —CH), 2975 (Aliph., C—H), 1641 (C═N), 1593 (Ar., C—C), 1454, 1396 (C—C), 1231 (Asym., C—O), 1165 (C—N), 1087, (Sym. C—O), 834. UV-Vis (DMSO): λmax (nm) 676, 609, 354. MS (MALDI-TOF): m/z 435.66 [M−4I]⁴⁺, 453.16 [M−4I+H₂O]⁴⁺

### Compound 219: Tetrakis [4-(4-(5-bromo-1H-benzo[d]imidazol-2-yl)phenoxy phthalocyaninato] copper(II) tetraiodide

FT-IR (UATR-TWO™) ν max/cm⁻¹: 3417 (NH), 3000 (Ar., —CH), 2974 (Aliph., C—H), 1658 (C═N), 1596 (Ar., C—C), 1470, 1401 (C—C), 1238 (Asym., C—O), 1167 (C—N), 1052, (Sym. C—O), 851. UV-Vis (DMSO): λmax (nm) 674, 606, 347. MS (MALDI-TOF): m/z 445.63 [M−4I]⁴⁺.

### Compound 231: Tetrakis [4-(4-(4-bromo-6-fluoro-1H-benzo[d]imidazol-2-yl)phenoxy phthalocyaninato] copper(II) tetraiodide

FT-IR (UATR-TWO™) ν max/cm⁻¹: 3411 (NH), 3008 (Ar., —CH), 2978 (Aliph., C—H), 1654 (C═N), 1591 (Ar., C—C), 1468, 1421 (C—C), 1232 (Asym., C—O), 1165 (C—N), 1087, (Sym. C—O), 851. UV-Vis (DMSO): λmax (nm) 675, 608, 353. MS (MALDI-TOF): m/z 464.76 [M−4I]⁴⁺.

### Compound 235: Tetrakis [4-(4-(6-bromo-4-chloro-1H-benzo[d]imidazol-2-yl)phenoxy phthalocyaninato] copper(II) tetraiodide

FT-IR (UATR-TWO™) ν max/cm⁻¹: 3411 (NH), 3014 (Ar., —CH), 2940 (Aliph., C—H), 1651 (C═N), 1592, 1508 (Ar., C—C), 1460, 1403 (C—C), 1236 (Asym., C—O), 1166 (C—N), 1094, (Sym. C—O), 850. UV-Vis (DMSO): λmax (nm) 676, 608, 351. MS (MALDI-TOF): m/z 479.79 [M−4I−1]⁴⁺.

### Compound 244: Tetrakis [4-(4-(5-chloro-1H-benzo[d]imidazol-2-yl)phenoxy phthalocyaninato] copper(II) tetraiodide

FT-IR (UATR-TWO™) ν max/cm⁻¹: 3438 (NH), 3007 (Ar., —CH), 2939 (Aliph., C—H), 1647 (C═N), 1529, 1513 (Ar., C—C), 1469, 1400 (C—C), 1238 (Asym., C—O), 1167 (C—N), 1097, (Sym. C—O), 939. UV-Vis (DMSO): λmax (nm) 677, 607, 359. MS (MALDI-TOF): m/z 400.66 [M−4I−1]⁴⁺.

### Antimicrobial Studies and Anticancer Studies

### In Vitro Studies

500 μM stock solutions of the samples were prepared in ultra-pure water (5% DMSO) and used at different concentrations in the biological assays.

### Organisms and Growth Conditions

Two bacterial strains were used for the PACT studies; S. aureus as a gram-(+) bacterium and E. coli as a gram-(−) negative bacterium. Stock cultures were grown aerobically in 6 mL of nutrient broth incubated at 37° C. on a rotary shaker (~200 rpm) to obtain bacteria cultures. The incubation was maintained until the optical density of the culture at 600 nm was determined to be between 0.6 and 0.8. The resulting bacterial solution was centrifuged to remove the broth, and bacterial cells were washed three times with PBS. The solution of the bacteria was prepared by diluting with PBS to obtain 10-5 M bacteria solutions corresponding to ~10⁹ and ~10⁸ colony forming units (CFU)/mL, respectively.

### Photodynamic Inactivation of Bacteria

Pc solutions were prepared by dissolving in water containing 5% DMSO and applied over the range of 0-10 μM for S. aureus and 0-15 μM for E. coli. The bacteria/photosensitizer mixtures were incubated in an oven equipped with a shaker for 30 min in the dark at 37° C. before plating. 100 μL of solution was taken from the suspension and was immediately inoculated on agar plates to determine the activity at 0 min without treatment. The number of colonies was then counted on each plate after 18 h of incubation at 37° C. Control treatments were performed in the absence of photosensitizer. All experiments were carried out three times. The data for CFU/mL were converted to the logarithmic form.

### In Vitro Dark Cytotoxicity

MCF-7 (human breast adenocarcinoma) cells were cultured in Dulbecco's modified Eagle's medium-10% fetal bovine serum (DMEM-FBS) and PSA (penicillin/streptomycin/Amphotericin B) at 37° C. in an incubator (5% CO₂) in 75 cm² vented flasks. The cultures were grown as a monolayer. The growth cells were transferred to well plates by trypsinizing with 0.25% trypsin-EDTA. The Pc stock concentrations were prepared by separately dissolving them in DMSO (1%) and diluting in water and the Pc gradient concentration was prepared by taking known aliquots from the stock and finally made up to marked volume with supplemented media with phenol red.

It was studied from 5 to 120 μM both in the dark. 100 μL each of the newly prepared gradient Pcs concentration of 0 μg/mL (control), 5 μg/mL, 10 μg/mL, 20 μg/mL, 40 μg/mL, 60 μg/mL 80 μg/mL, 100 μg/mL and 120 μg/mL were administered on the incubated seeded cells. The 96 well cell culture plates containing the cells and the Pcs were incubated at 37° C. and 5% CO₂ in the dark for 24 h. After 24 h treatment, the wells were rinsed with 100 μL DPBS. Cell survival was expressed as percentage of control cells (cells without Pcs). Post treatment cell viability was measured using the cell proliferation neutral red reagent (WST-1 assay) on a Synergy 2 multi-mode microplate reader (BioTek®) at a wavelength of 450 nm according to the following equation 1.

$$\% Cell Viability = \frac{Absorbance of samples at 450 nm}{Absorbance of control at 450 nm} \qquad ( 1 )$$

The results of all compounds are separated into separated discussions for 6 chemically distinct series of compounds, which are as follows:
Series 1: Compounds 193, 200 and 201
Series 2: Compounds 207, 208 and 209
Series 3: Compounds 217, 218 and 219
Series 4: Compounds 229, 230 and 231
Series 5: Compounds 233, 234 and 235
Series 6: Compounds 242, 243 and 244

### Results

Series 1: Antimicrobial and anticancer studies of compounds 193, 200 and 201

### Compound 193

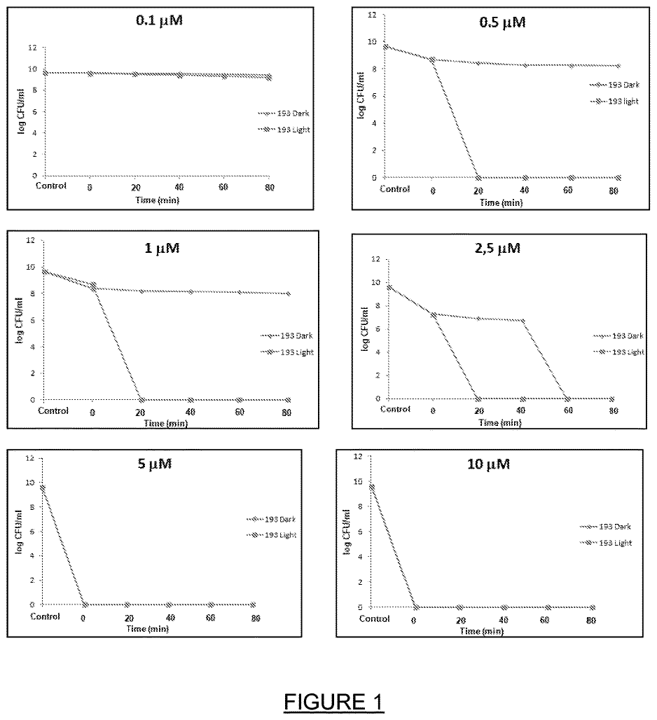

As seen from FIG. 1, compound 193 showed photocytotoxicities against S. aureus after irradiation 20 min at 0.5, 1 and 2.5 μM concentrations with the log reduction of 9.63. Compound 193 showed dark toxicities at 0 min (as soon as mix the drug with bacteria solution) at 5 and 10 μM concentrations with the log reduction of 9.63.

### Compound 200

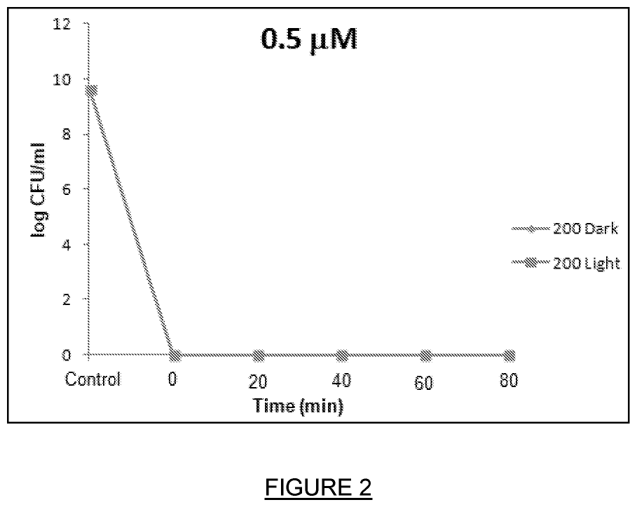

As seen from FIG. 2, compound 200 showed complete destruction at 0.5 μM concentration, and was therefore not performed at any other concentration values.

Compound 200 showed dark toxicities at 0 min (as soon as mix the drug with bacteria solution) at 0.5-10 μM concentration with the log reduction of 9.63.

### Compound 201

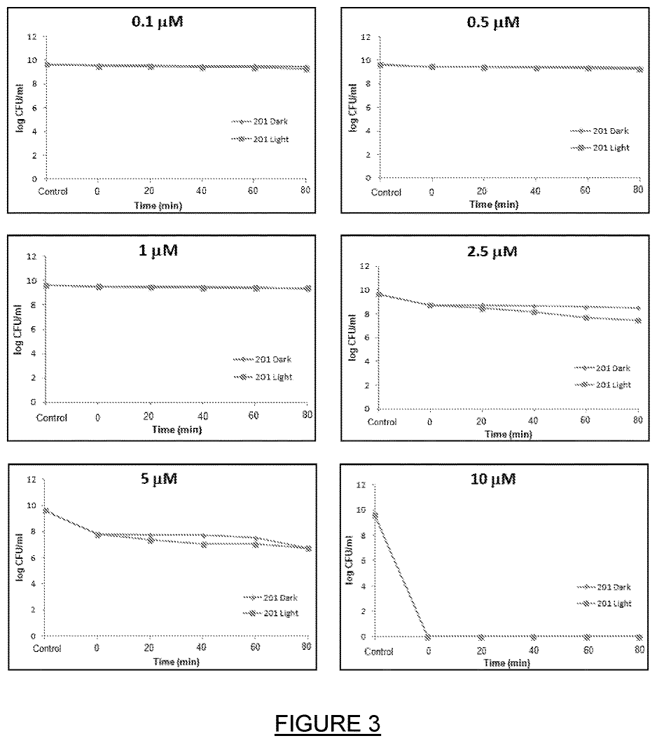

As seen from FIG. 3, compound 201 showed dark toxicities at 0 min (as soon as mix the drug with bacteria solution) at 10 μM concentration with the log reduction of 9.63.

The concentration studies for compounds 193, 200 and 201 are shown in FIG. 4(a) and in FIG. 4(b). The logarithmic reduction, as per Table 1 below, of FIG. 4(a) S. aureus, and FIG. 4(b) E. coli in the presence of compounds 193, 200, 201 in the dark, treated in each case with 0-10 μM for S. aureus and 0-15 μM for E. coli of the photosensitizer dyes (0 μM=Control).

TABLE 1
Logarithmic reduction values corresponding
to concentrations study at 0 min.

| | |
|---|---|
| Staph | Ecoli |

| Compounds | μM | log CFU/ml | μM | log CFU/ml |
|---|---|---|---|---|
| 193 | 4 | 9.61 | 1 | 8.79 |
| 200 | 0.5 | | 1 | |
| 201 | 8 | | 12.5 | |

### Visualization of Bacteria Cell Death of Compounds 193, 200 and 201

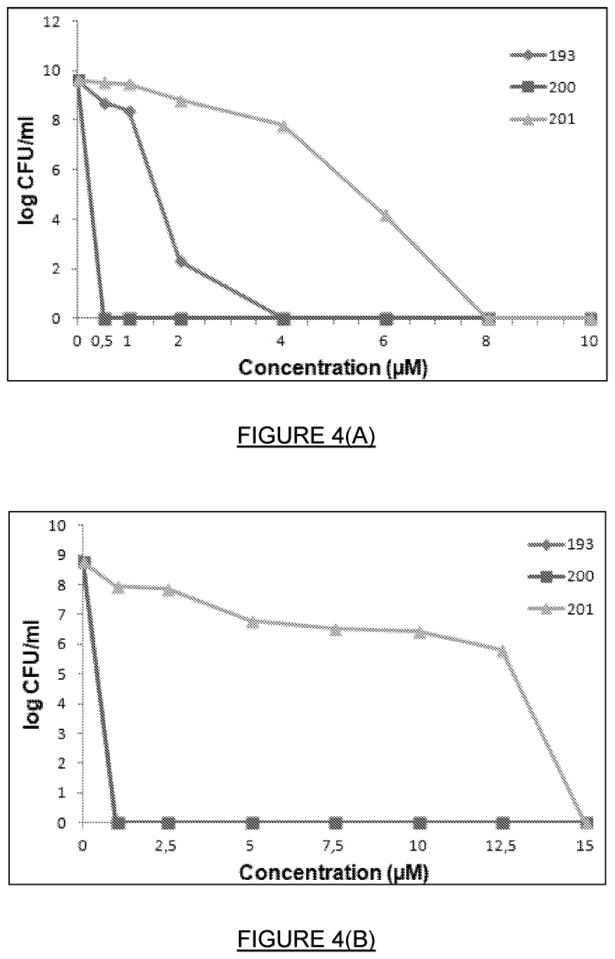

The visualization of bacteria cell death of compounds 193, 200 and 201 is shown in FIG. 5(a) to FIG. 5(d). In these Figures, the agar plate micrographs are shown depicting the (a) S. aureus colonies 5% DMSO in PBS as control, (b) 5, 0.5 10 μM of 193, 200, 201 at 0. min., (b) E. coli colonies 5% DMSO in PBS as control, and (d) 1, 1, 12.5 μM of 193, 200, 201 at 0. min.

### Series 1: Anticancer Studies of Compounds 193, 200 and 201

The study was performed for cancer cells and the anticancer results for compounds 193, 200 and 201 are shown in Table 2. Future studies involving human cells are underway.

TABLE 2
Anticancer results for compounds 193, 200 and 201

| Compounds | Min. Death conc. (μM) | Cell death (%) |
|---|---|---|
| 193 | 40 | 72.7 |
| 200 | 40 | 80.9 |
| 201 | 120 | 45.4 |

From the above, the optimal concentrations are 40 μM for compound 193, 40 μM for compound 200, and 120 μM for compound 201 to be able to kill the cancer cells with 72.7%, 80.9%, 45.4%, respectively.

### Series 2: Antimicrobial and Anticancer Studies of Compounds 207, 208 and 209

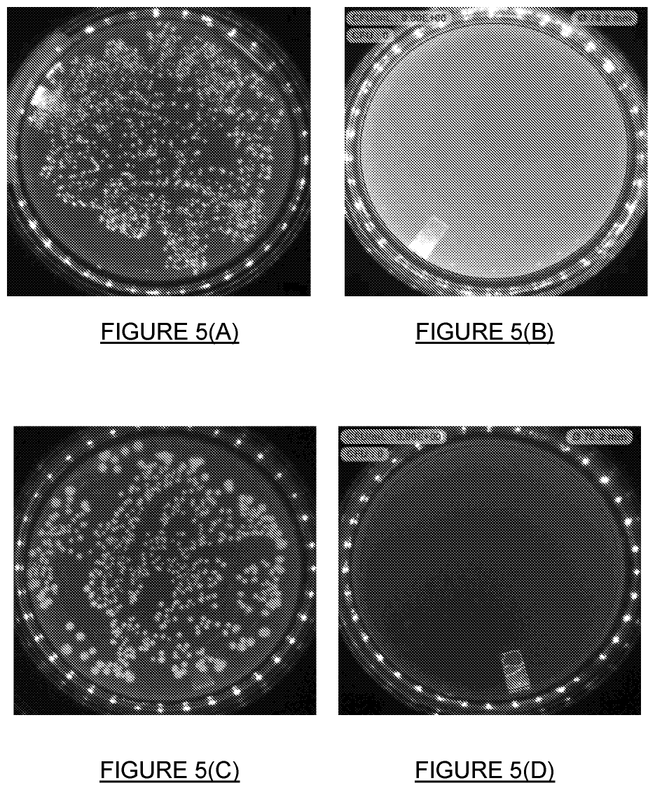

The concentration studies for compounds 207, 208 and 209 are shown in FIG. 6(a) and in FIG. 6(b).

The logarithmic reduction, as per Table 3 below, of FIG. 6(a) S. aureus, and FIG. 6(b) E. coli in the presence of 207, 208, 209 in the dark, treated in each case with 0-10 μM for S. aureus and 0-15 μM for E. coli of the photosensitizer dyes (0 μM=Control).

TABLE 3
Logarithmic reduction values corresponding
to concentrations study at 0 min

| | |
|---|---|
| Staph | Ecoli |

| Compounds | μM | log CFU/ml | μM | log CFU/ml |
|---|---|---|---|---|
| 207 | 4 | 9.31 | 2.5 | 8.79 |
| 208 | 2 | | 1 | |
| 209 | 10 | | 2.5 | |

### Series 2: Visualization of Bacteria Cell Death of Compounds 207, 208 and 209

The visualization of bacteria cell death of compounds 207, 208 and 209 is shown in FIG. 7(a) to FIG. 7(d). In these Figures, the agar plate micrographs are shown depicting the (a) S. aureus colonies 5% DMSO in PBS as control, (b) 4, 2, 10 μM of 207, 208, 209 at 0. min., (b) E. coli colonies 5% DMSO in PBS as control, (d) 2.5, 1, 2.5 μM of compounds 207, 208, 209 at 0. min.

### Series 2: Anticancer Studies of Compounds 207, 208 and 209

The anticancer results for compounds 207, 208 and 209 are shown in Table 4 below.

TABLE 4
Anticancer results for complexes

| Compounds | Min. Death conc. (μM) | Cell death (%) |
|---|---|---|
| 207 | 120 | 38.3 |
| 208 | 20 | 78.4 |
| 209 | 20 | 80.4 |

### Series 3: Antimicrobial and Anticancer Studies of Compounds 217, 218 and 219

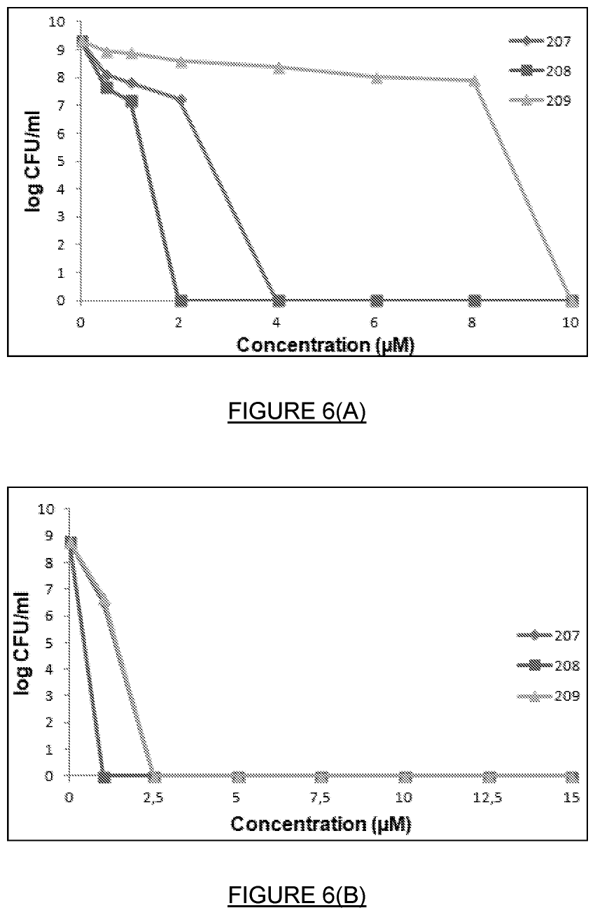

The concentration studies for compounds 217, 218 and 219 are shown in FIG. 8(a) and in FIG. 8(b). The logarithmic reduction, as per Table 5 below, of FIG. 8(a) S. aureus, and FIG. 8(b) E. coli in the presence of 217, 218, 219 in the dark, treated in each case with 0-10 μM for S. aureus and 0-15 μM for E. coli of the photosensitizer dyes (0 μM=Control).

TABLE 5
Logarithmic reduction values corresponding
to concentrations study at 0 min

| | |
|---|---|
| Staph | Ecoli |

| Compounds | μM | log CFU/ml | μM | log CFU/ml |
|---|---|---|---|---|
| 217 | 1 | 9.15 | 5 | 8.78 |
| 218 | 0.5 | | 1 | |
| 219 | 10 | | 12.5 | |

### Series 3: Visualization of Bacteria Cell Death for Compounds 217, 218 and 219

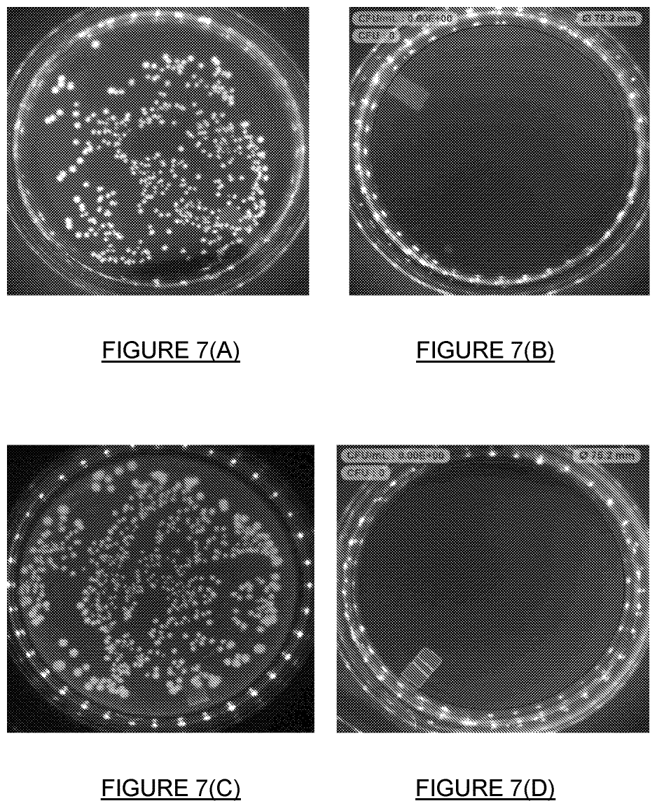

The visualization of bacteria cell death of compounds 217, 218 and 219 is shown in FIG. 9(a) to FIG. 9(d). In these Figures, the agar plate micrographs are shown depicting the (a) S. aureus colonies 5% DMSO in PBS as control, (b) 1, 0.5, 10 μM of 217, 218, 219 at 0. min., (b) E. coli colonies 5% DMSO in PBS as control, and (d) 5, 1, 12.5 μM of 217, 218, 219 at 0. min.

### Series 3: Anticancer Studies of Compounds 217, 218 and 219

The anticancer results for compounds 217, 218 and 219 are shown in Table 6 below.

TABLE 6
Anticancer results for complexes

| Compounds | Min. Death conc. (μM) | Cell death (%) |
|---|---|---|
| 217 | 5 | 40.1 |
| 218 | 10 | 56.6 |
| 219 | 10 | 36.9 |

### Series 4: Antimicrobial and Anticancer Studies of Compounds 229, 230 and 231

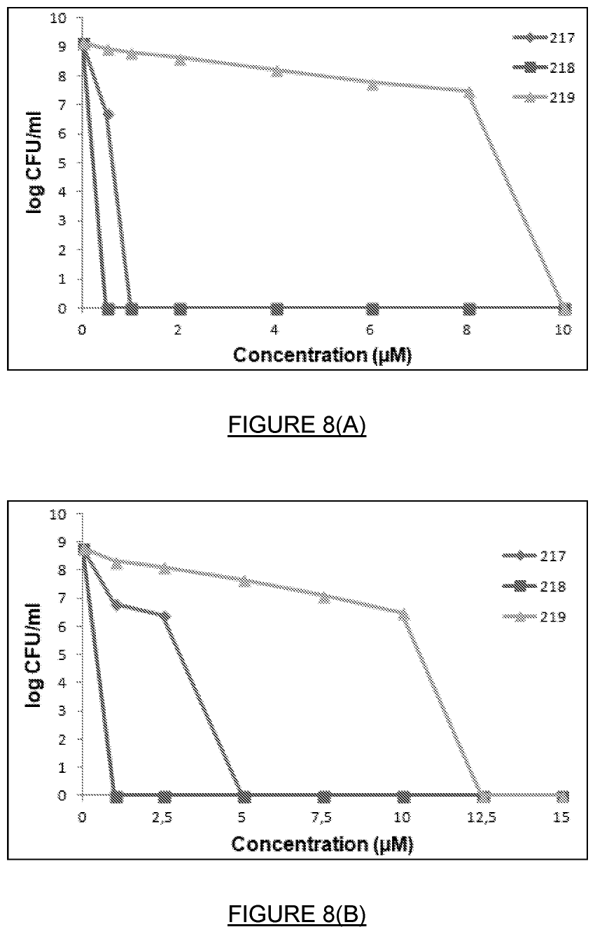

The concentration studies for compounds 229, 230 and 231 are shown in FIG. 10(a) and in FIG. 10(b). The logarithmic reduction, as per Table 7 below, of FIG. 10(a) S. aureus, and FIG. 10(b) E. coli in the presence of 229, 230, 231 in the dark, treated in each case with 0-10 μM for S. aureus and 0-15 μM for E. coli of the photosensitizer dyes (0 μM=Control).

TABLE 7
Logarithmic reduction values corresponding
to concentrations study at 0 min

| | |
|---|---|
| Staph | Ecoli |

| Compounds | μM | log CFU/ml | μM | log CFU/ml |
|---|---|---|---|---|
| 229 | 0.5 | 9.25 | 10 | 8.79 |
| 230 | 0.5 | | 1 | |
| 231 | 0.5 | | 12.5 | |

### Series 4: Visualization of Bacteria Cell Death of Compounds 229, 230 and 231

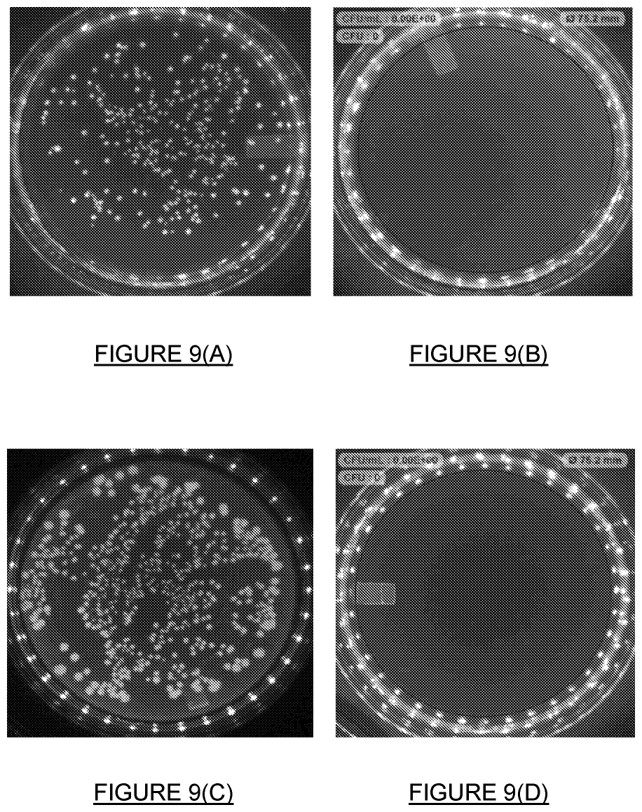

The visualization of bacteria cell death of compounds 229, 230 and 231 is shown in FIG. 11(a) to FIG. 11(d). In these Figures, the agar plate micrographs are shown depicting the (a) S. aureus colonies 5% DMSO in PBS as control, (b) 0.5, 0.5, 0.5 μM of 229, 230, 231 at 0. min., (b) E. coli colonies 5% DMSO in PBS as control, and (d) 10, 1, 12.5 μM of 229, 230, 231 at 0. min.

### Series 4: Anticancer Studies of Compounds 229, 230 and 231

The anticancer results for compounds 229, 230 and 231 are shown in Table 8 below.

TABLE 8
Anticancer results for complexes

| Compounds | Min. Death conc. (μM) | Cell death (%) |
|---|---|---|
| 229 | 10 | 39.4 |
| 230 | 5 | 54.9 |
| 231 | 5 | 56.08 |

### Series 5: Antimicrobial and Anticancer Studies of Compounds 233, 234 and 235

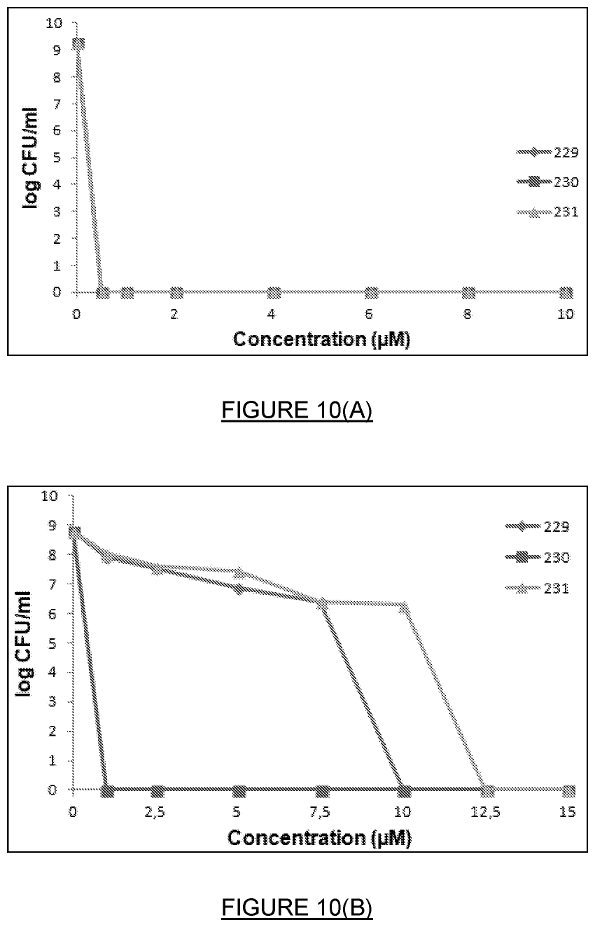

The concentration studies for compounds 233, 234 and 235 are shown in FIG. 12(a) and in FIG. 12(b). The logarithmic reduction, as per Table 9 below, of FIG. 12(a) S. aureus, and (b) E. coli in the presence of 233, 234, 235 in the dark, treated in each case with 0-10 μM for S. aureus and 0-15 μM for E. coli of the photosensitizer dyes (0 μM=Control).

TABLE 9
Logarithmic reduction values corresponding
to concentrations study at 0 min

| | |
|---|---|
| Staph | Ecoli |

| Compounds | μM | log CFU/ml | μM | log CFU/ml |
|---|---|---|---|---|
| 233 | 4 | 9.25 | 1 | 8.79 |
| 234 | 0.5 | | 2.5 | |
| 235 | 10 | | 12.5 | |

### Series 5: Visualization of Bacteria Cell Death of Compounds 233, 234 and 235

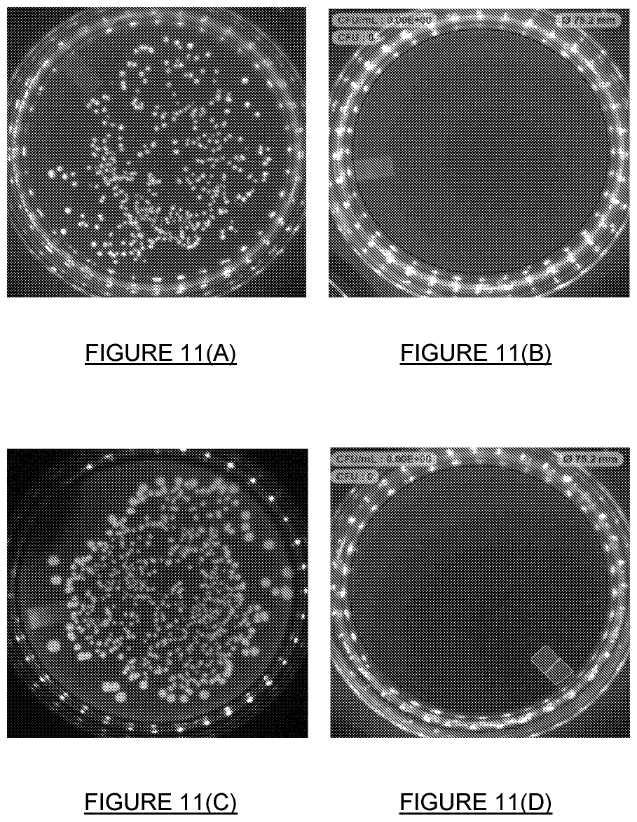

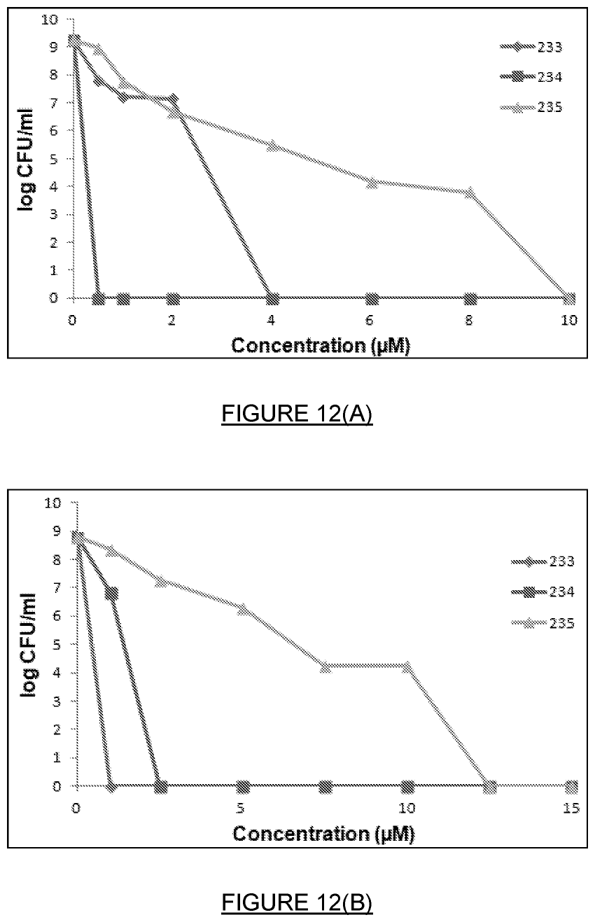

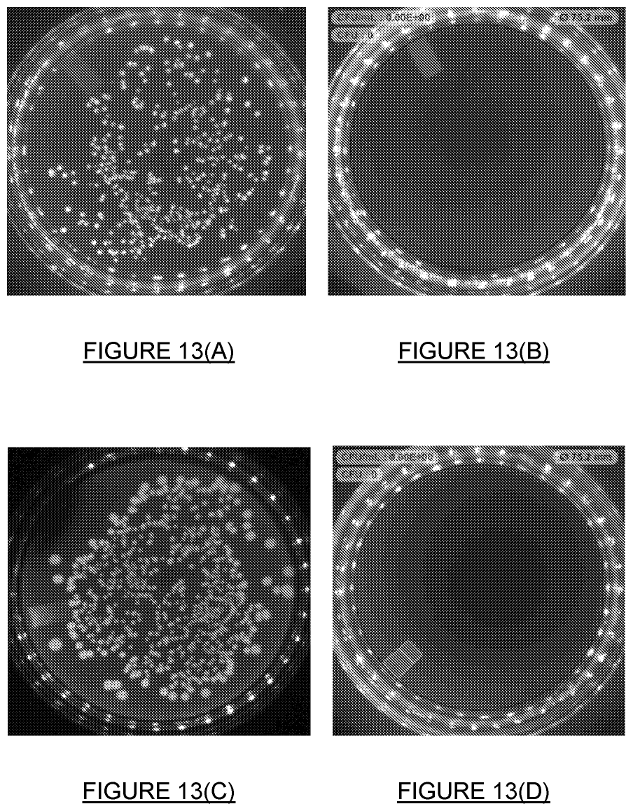

The visualization of bacteria cell death of compounds 233, 234 and 235 is shown in FIG. 13(a) to FIG. 13(d). In these Figures, the agar plate micrographs are shown depicting the (a) S. aureus colonies 5% DMSO in PBS as control, (b) 4, 0.5, 10 μM of 233, 234, 235 at 0. min., (b) E. coli colonies 5% DMSO in PBS as control, (d) 1, 2.5, 12.5 μM of 233, 234, 235 at 0. min.

### Series 5: Anticancer Studies of Compounds 233, 234 and 235

The anticancer results for compounds 233, 234 and 235 are shown in Table 10 below.

TABLE 10
Anticancer results for complexes

| Compounds | Min. Death conc. (μM) | Cell death (%) |
|---|---|---|
| 233 | 60 | 91.7 |
| 234 | 60 | 90.4 |
| 235 | 10 | 34.8 |

### Series 6: Antimicrobial and Anticancer Studies of Compounds 242, 243 and 244

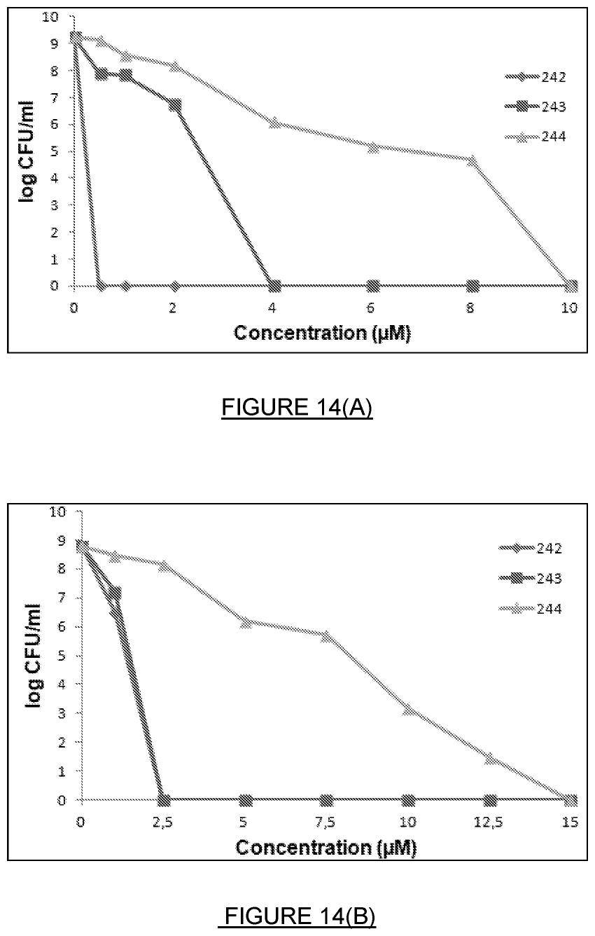

The concentration studies for compounds 242, 243 and 244 are shown in FIG. 14(a) and in FIG. 14(b). The logarithmic reduction, as per Table 11 below, of FIG. 14(a) S. aureus, and (b) E. coli in the presence of 242, 243, 244 in the dark, treated in each case with 0-10 μM for S. aureus and 0-15 μM for E. coli of the photosensitizer dyes (0 μM=Control).

TABLE 11
Logarithmic reduction values corresponding
to concentrations study at 0 min

| | |
|---|---|
| Staph | Ecoli |

| Compounds | μM | log CFU/ml | μM | log CFU/ml |
|---|---|---|---|---|
| 242 | 0.5 | 9.24 | 2.5 | 8.79 |
| 243 | 4 | | 2.5 | |
| 244 | 10 | | 15 | |

### Series 6: Visualization of Bacteria Cell Death of Compounds 242, 243 and 244

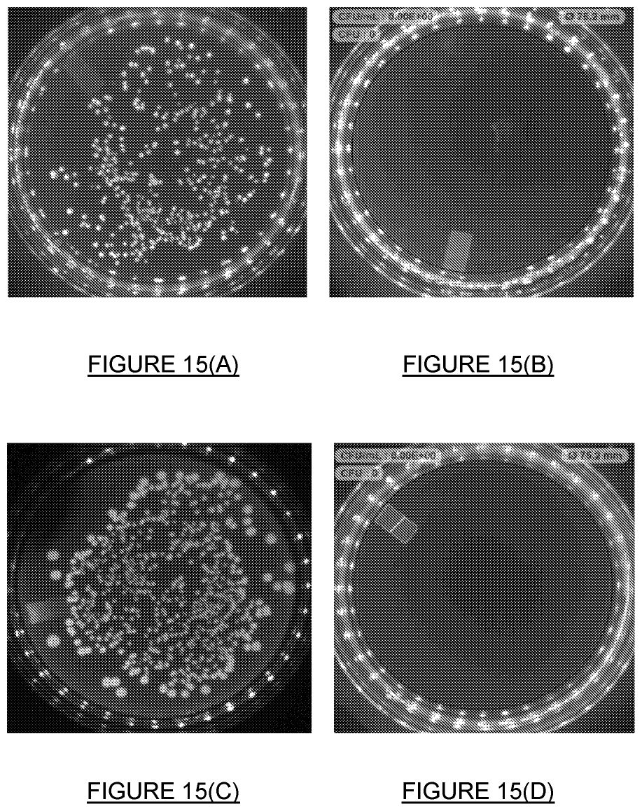

The visualization of bacteria cell death of compounds 242, 243, 244 is shown in FIG. 15(a) to FIG. 15(d). In these Figures, the agar plate micrographs are shown depicting the (a) S. aureus colonies 5% DMSO in PBS as control, (b) 0.5, 4, 10 μM of 242, 243, 244 at 0. min, (b) E. coli colonies 5% DMSO in PBS as control, and (d) 2.5, 2.5, 15 μM of 242, 243, 244 at 0. min.

### Series 6: Anticancer Studies of Compounds 242, 243 and 244

The anticancer results for compounds 242, 243, 244 are shown in Table 12 below.

TABLE 12
Anticancer results for complexes

| Compounds | Min. Death conc. (μM) | Cell death (%) |
|---|---|---|
| 242 | 120 | 49.8 |
| 243 | 120 | 62.4 |
| 244 | 80 | 90.8 |

### Spectroscopic Characterisation and Discussion

The estimated chemical structures were confirmed with the spectroscopic methods such as, ¹H-NMR, ¹³C-NMR, UV/Vis, FT-IR and MS analysis. All spectral data showed that the synthesized compounds are in the expected structure.

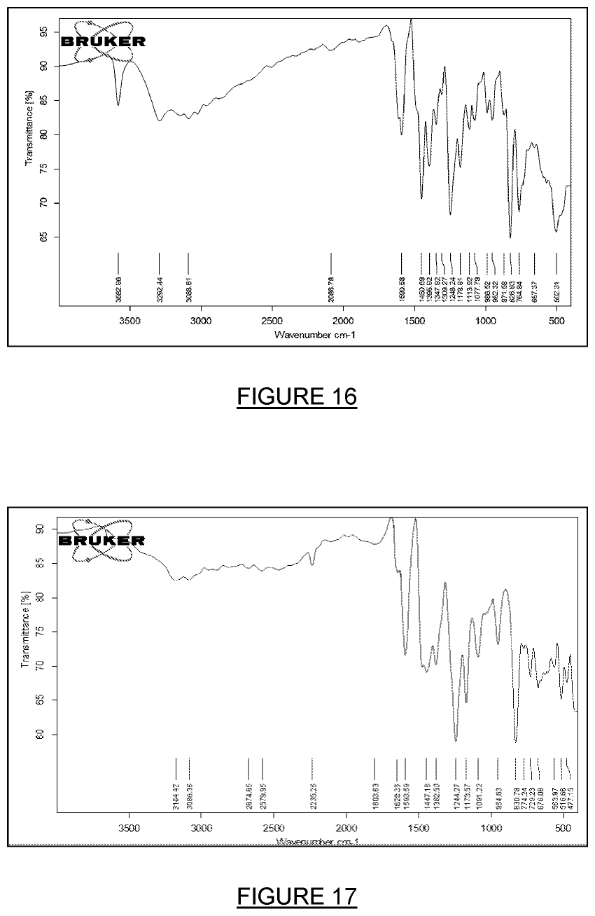

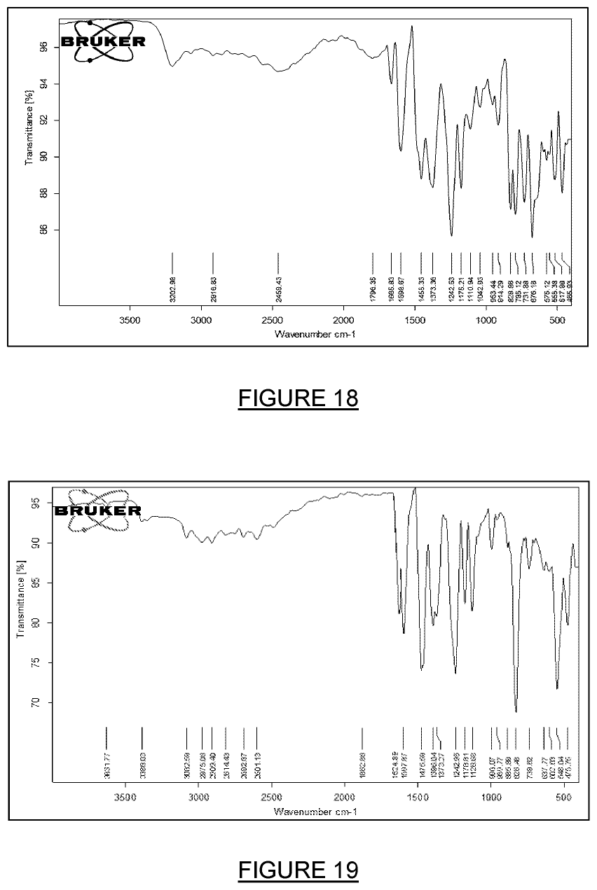

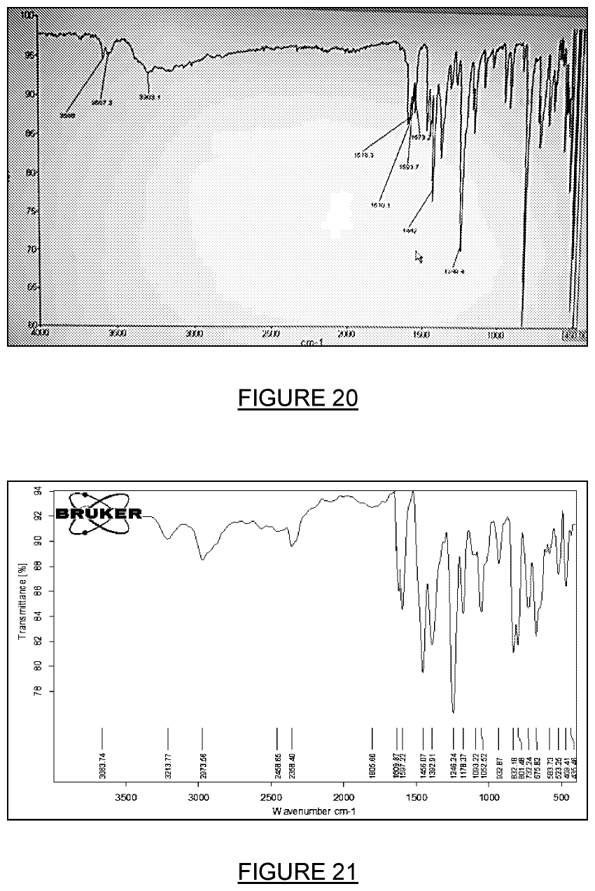

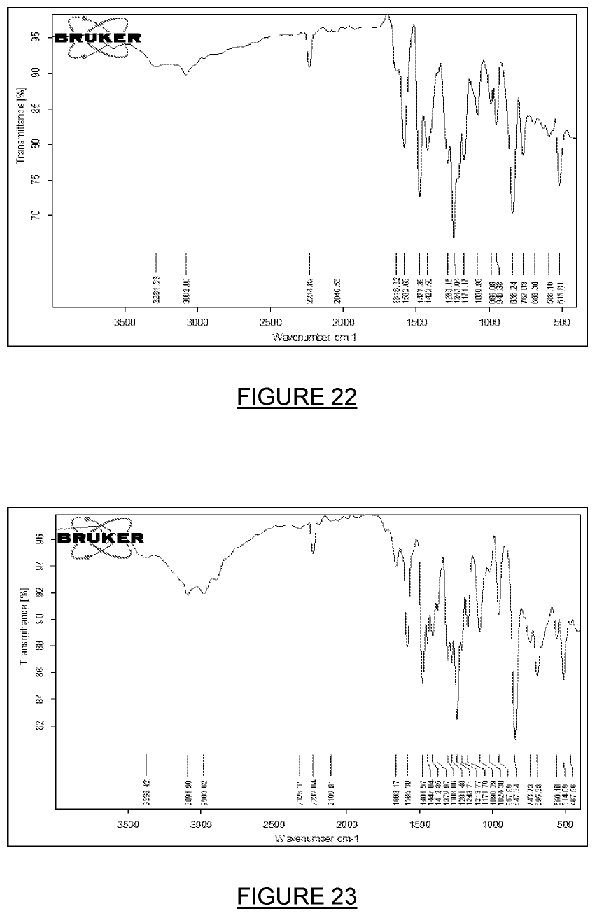

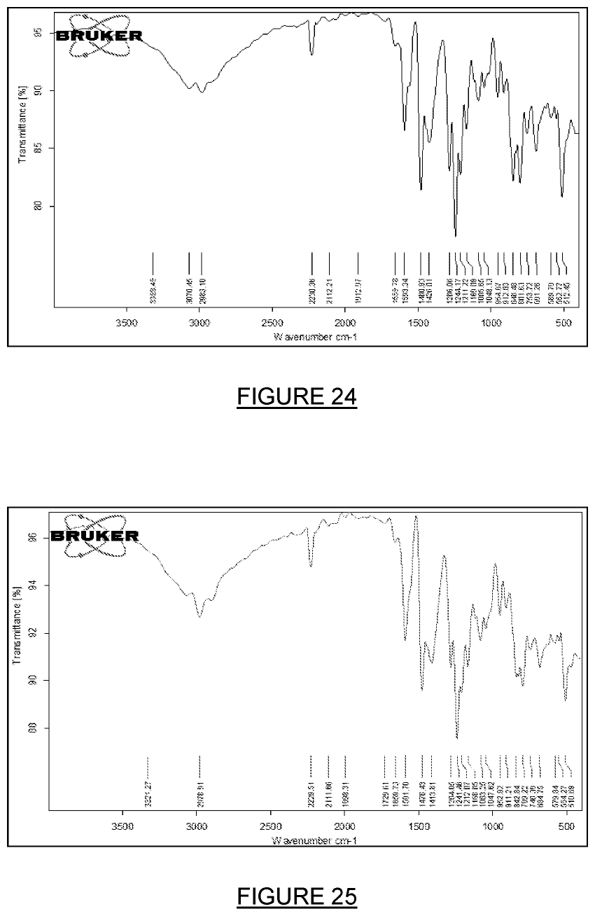

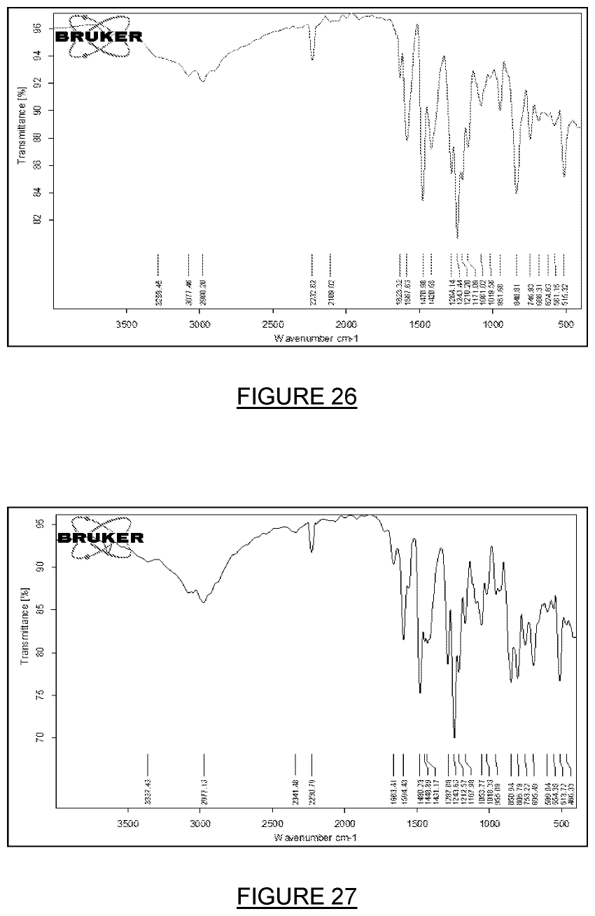

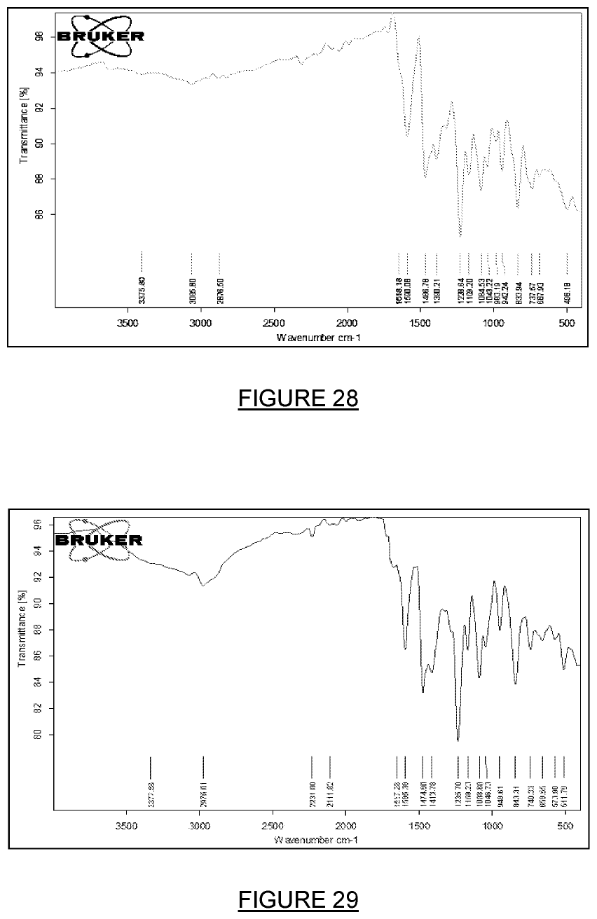

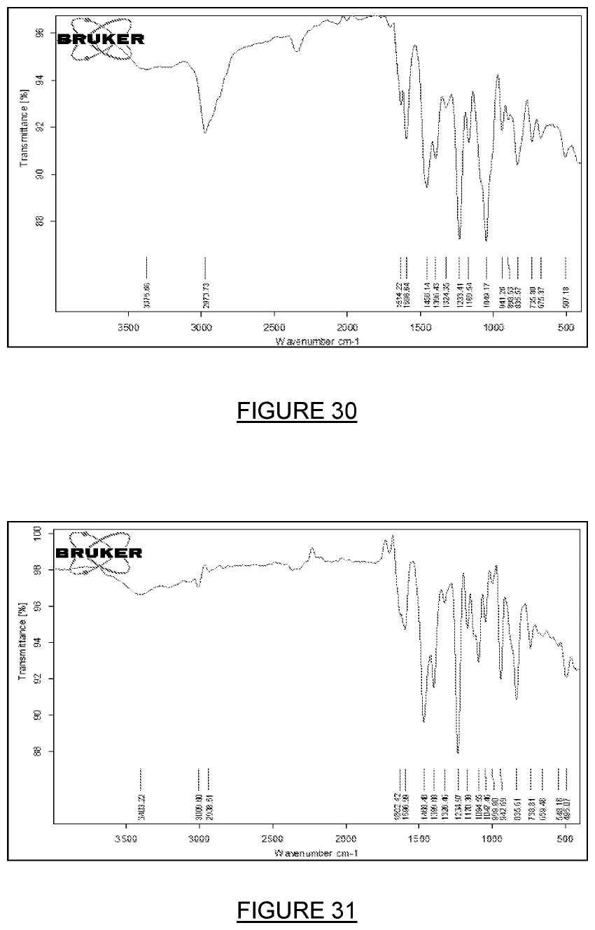

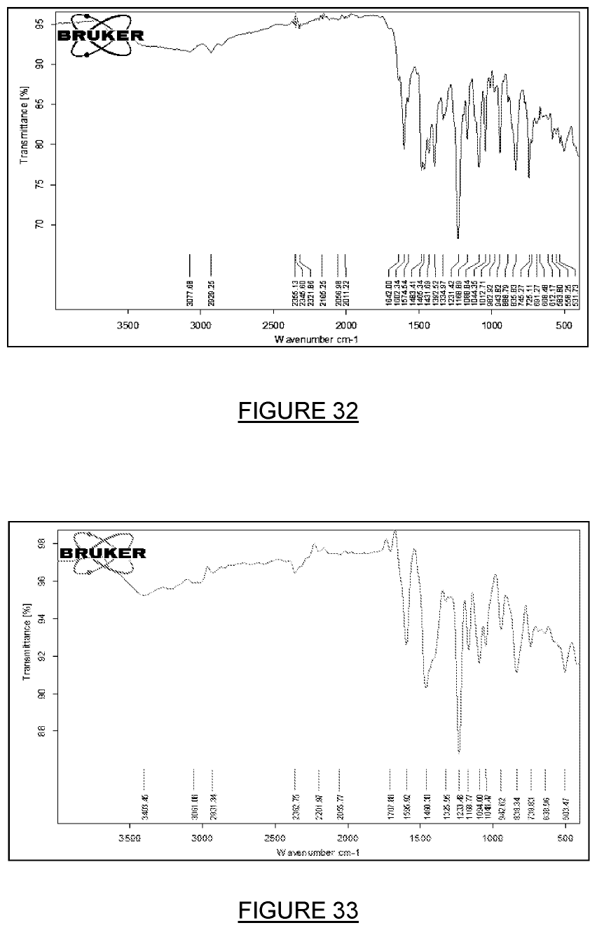

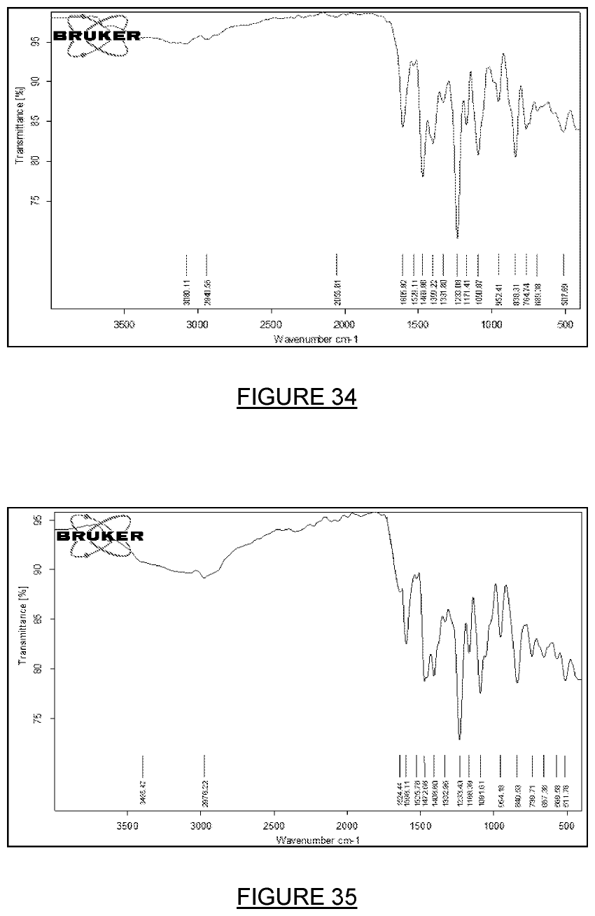

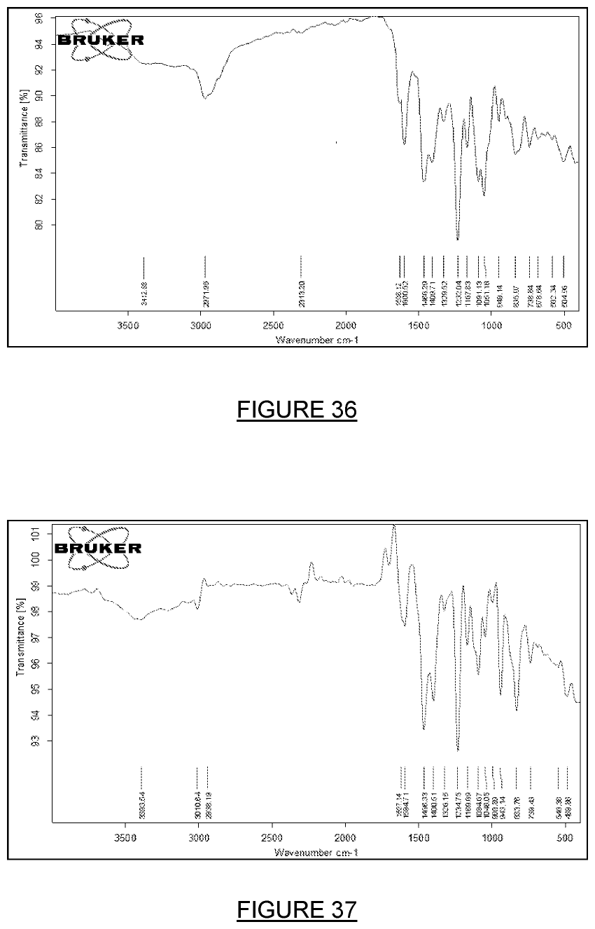

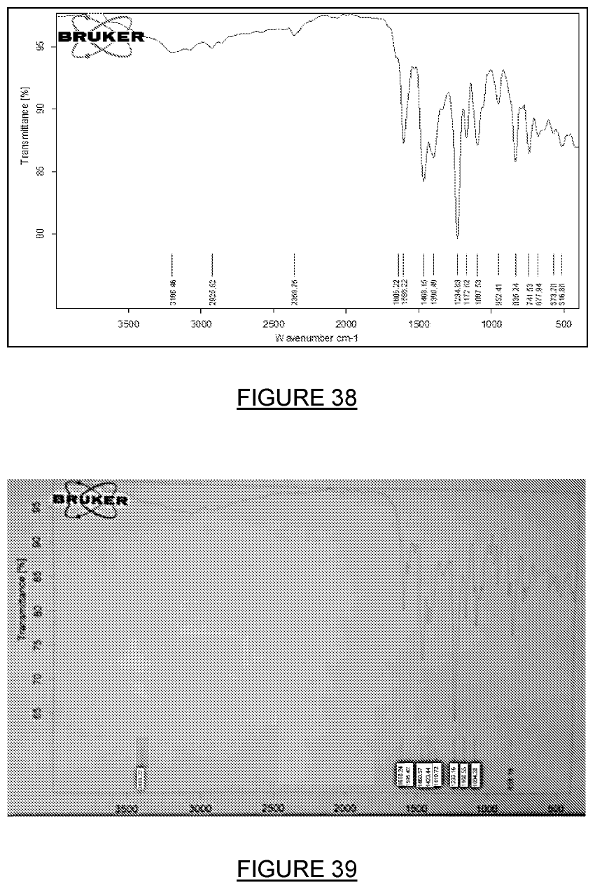

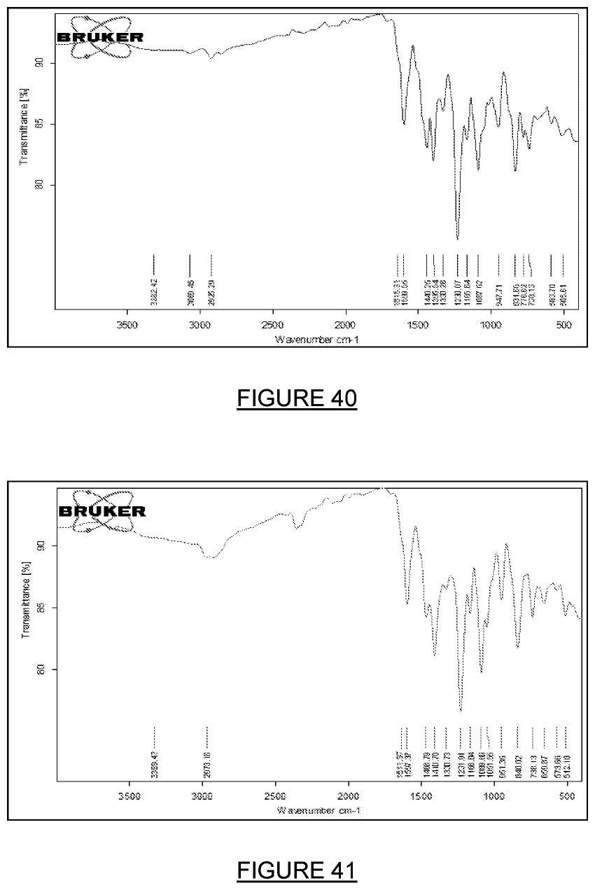

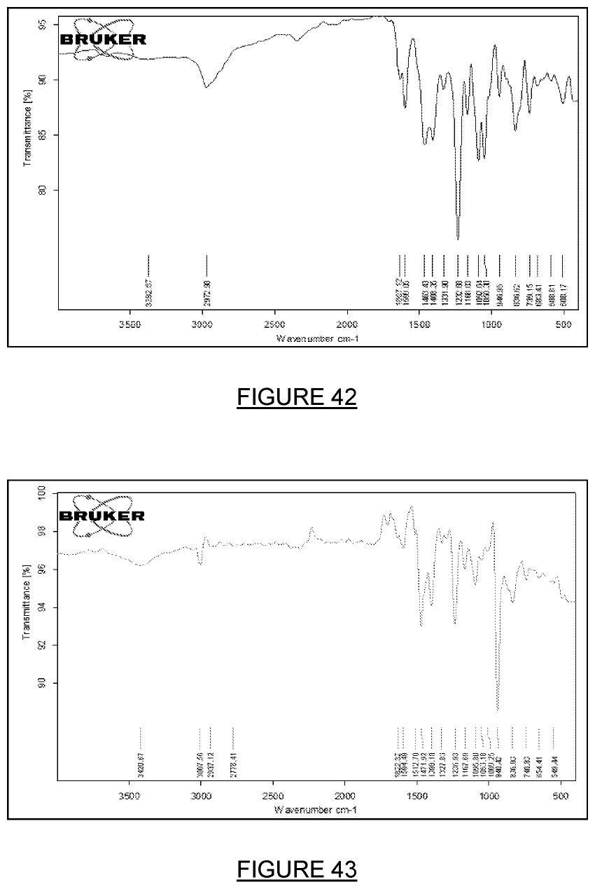

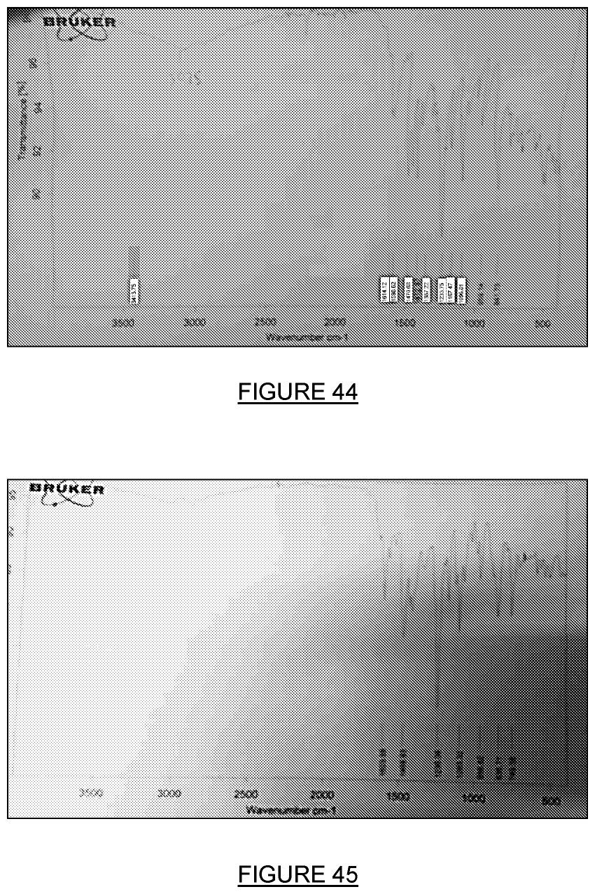

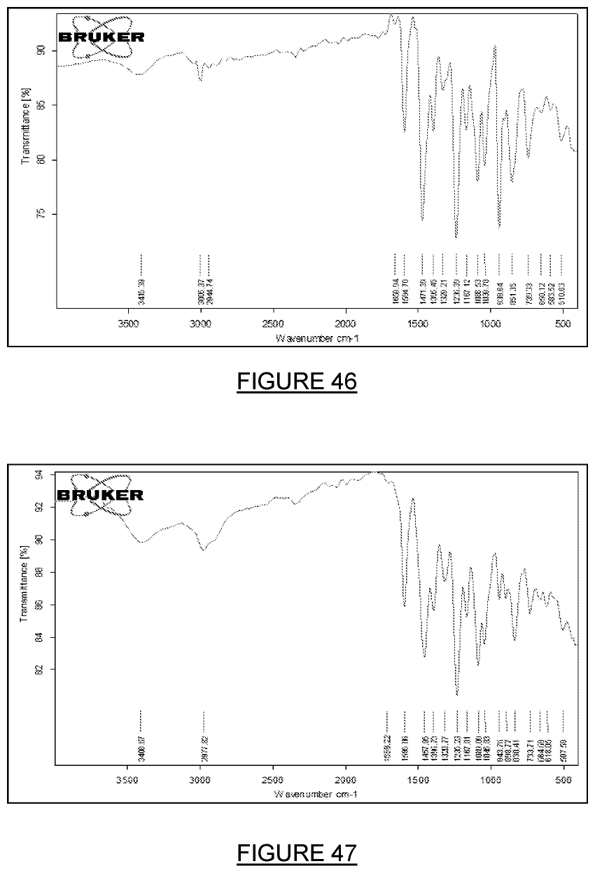

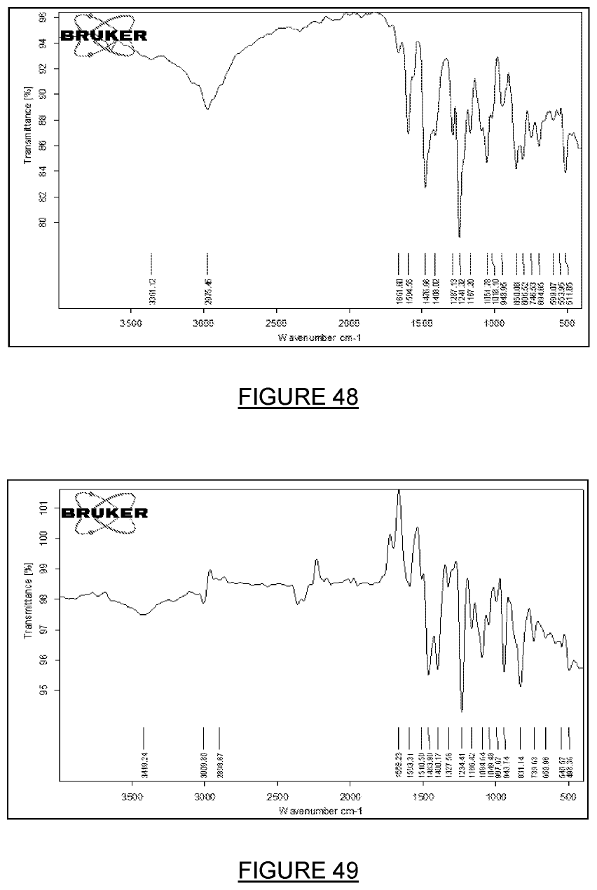

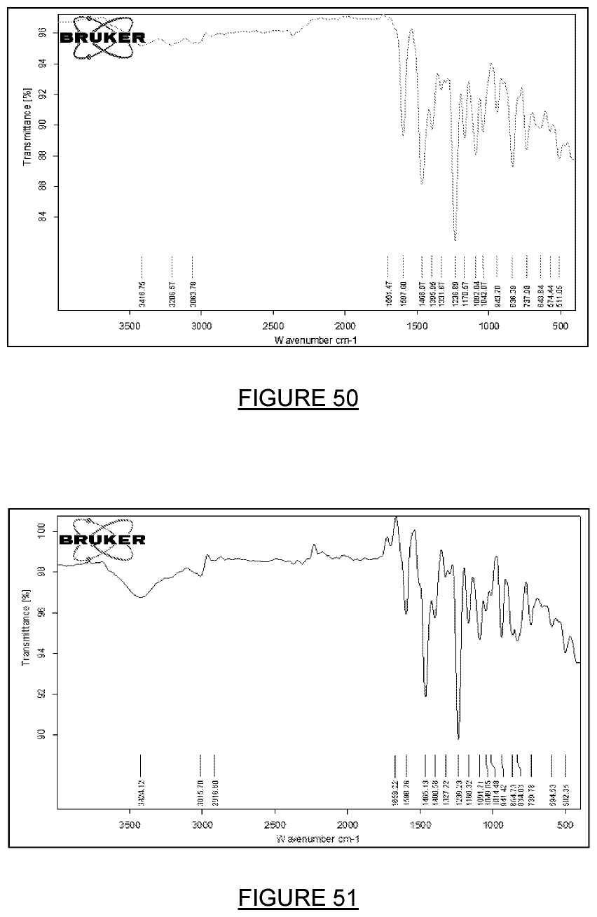

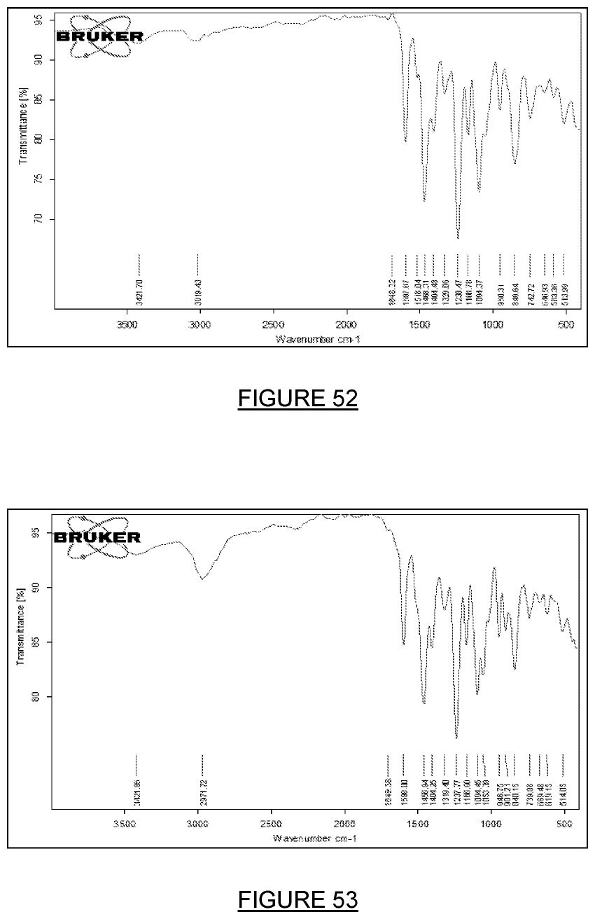

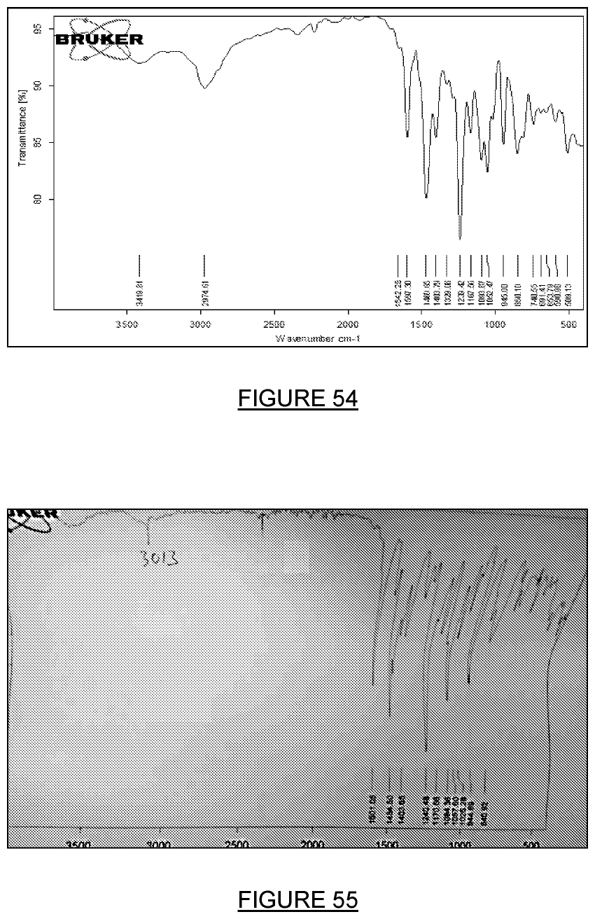

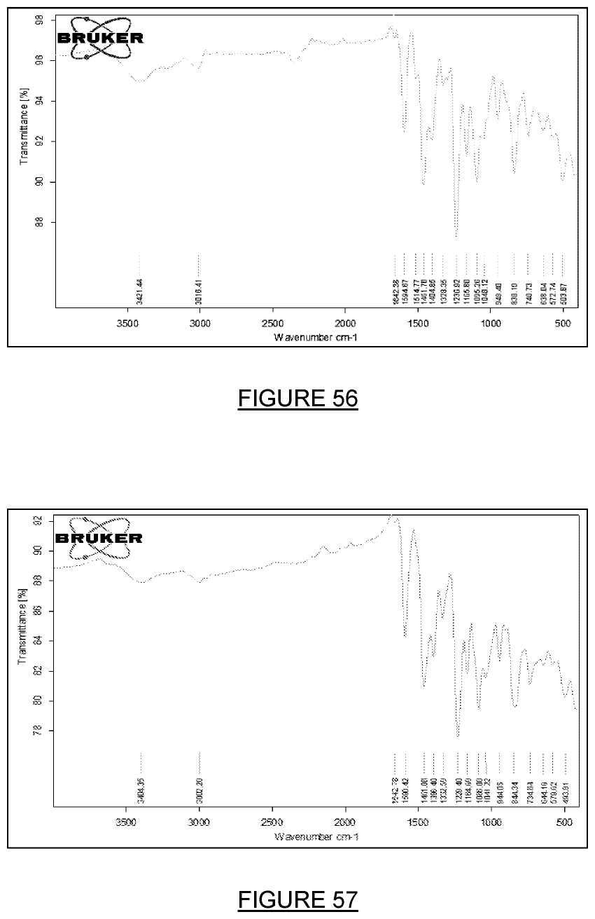

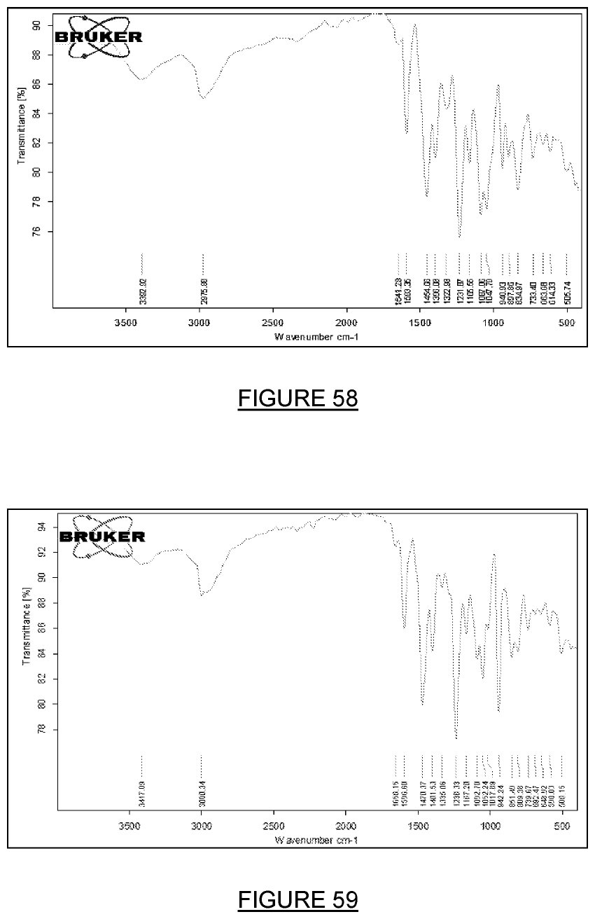

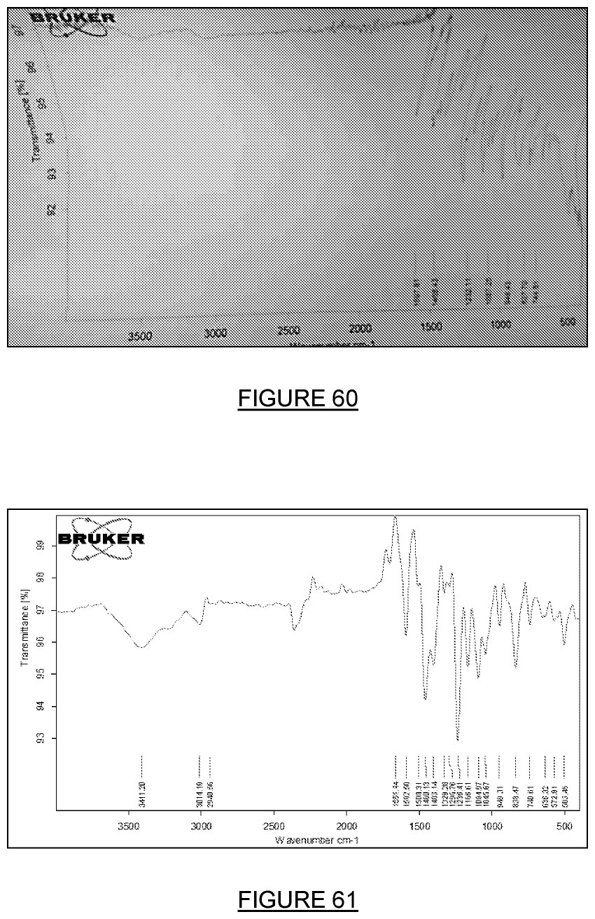

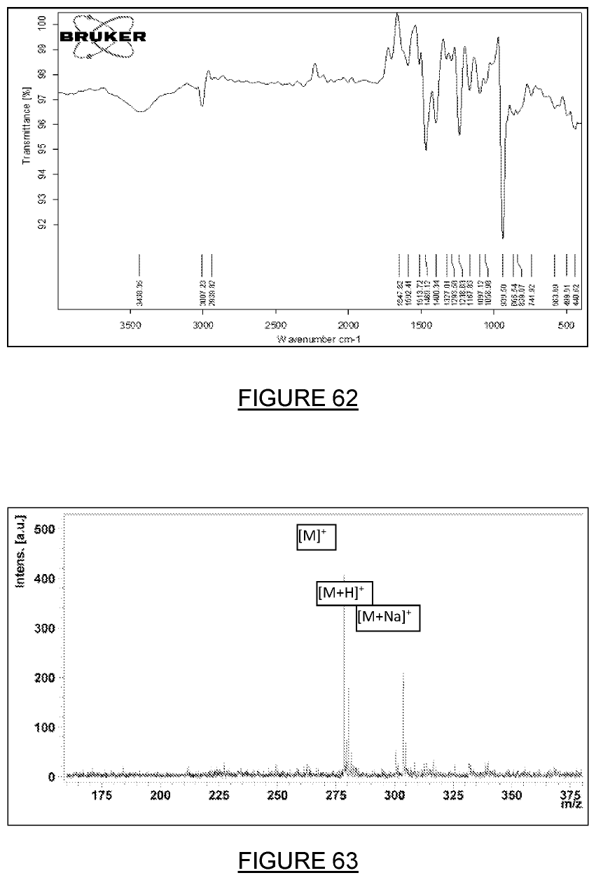

The FT-IR spectra for the compounds are shown in FIG. 16 to FIG. 62.

In the FT-IR spectrum of 180, 181, 182, 184, 185 and 215 the significant difference is that carbonyl vibration of the aldehyde functional group and NH₂ groups disappeared and (—C═N) band was observed at 1620 cm⁻¹, 1620 cm⁻¹, 1665 cm⁻¹, 1624 cm⁻¹, 1618 cm⁻¹, 1609 cm⁻¹, respectively. The —OH and —NH vibration bands were observed at 3582 cm⁻¹ and 3292 cm⁻¹ for 180, 3104-2579 (NH, Ar. CH, intermolecular H bonds) for 181, 3202-2549 (NH, Ar. CH, intermolecular H bonds) for 182, 3631 cm⁻¹ and 3388 cm⁻¹ for 184, 3586 cm⁻¹ and 3557 cm⁻¹ for 185, 3663 cm⁻¹ and 3213 for 215. The presence of new vibration bands at 2234 cm⁻¹ and 1243 cm⁻¹ for 186, 2232 cm⁻¹ and 1243 cm⁻¹ for 187, 2230 cm⁻¹ and 1246 cm⁻¹ for 188, 2229 cm⁻¹ and 1241 cm⁻¹ for 190, 2232 cm⁻¹ and 1240 cm⁻¹ for 191, 2230 cm⁻¹ and 1243 cm⁻¹ for 216, referring to —C═N and Ar—O—R, which indicate the existence of phthalonitrile for all samples.

The tetramerization of the phthalonitrile derivatives into the corresponding metallo phthalocyanines (189, 203, 211, 221, 225, 237, 197, 204, 212, 222, 226, 238, 198, 205, 213, 223, 227 and 239) was approved with the disappearance of the nitrile (C═N) peaks.

The FT-IR spectra were similar for all Pcs. The NH and C=N bands coming from the benzimidazole units appeared at 3375 cm⁻¹ and 1618 cm⁻¹ for 189, 3377 cm⁻¹ and 1612 cm⁻¹ for 203, 3375 cm⁻¹ and 1614 cm⁻¹ for 211, 3403 cm⁻¹ and 1602 cm⁻¹ for 221, 3413 cm⁻¹ and 1642 cm⁻¹ for 225, 3403 cm⁻¹ and 1608 cm⁻¹ for 237, 3389 cm⁻¹ and 1605 cm⁻¹ for 197, 3405 cm⁻¹ and 1624 cm⁻¹ for 204, 3412 cm⁻¹ and 1608 cm⁻¹ for 212, 3393 cm⁻¹ and 1607 cm⁻¹ for 222, 3198 cm⁻¹ and 1605 cm⁻¹ for 226, 3403 cm⁻¹ and 1608 cm⁻¹ for 238, 3382 cm⁻¹ and 1615 cm⁻¹ for 198, 3389 cm⁻¹ and 1611 cm⁻¹ for 205, 3392 cm⁻¹ and 1602 cm⁻¹ for 213, 3420 cm⁻¹ and 1622 cm⁻¹ for 223, 3396 cm⁻¹ and 1614 cm⁻¹ for 227, 3399 cm⁻¹ and 1608 cm⁻¹ for 239.

After the quaternization reactions to obtain 193, 200, 201, 207, 208, 209, 217, 218, 219, 229, 230, 231, 233, 234, 235, 242, 243 and 244, the imine band shifted to higher frequency. The new C=N bands took place at 1659 cm⁻¹ for 193, 1659 cm⁻¹ for 207, 1661 cm⁻¹ for 217, 1659 cm⁻¹ for 229, 1661 cm⁻¹ for 233, 1659 cm⁻¹ for 242, 1648 cm⁻¹ for 200, 1649 cm⁻¹ for 208, 1642 cm⁻¹ for 218, 1645 cm⁻¹ for 230, 1642 cm⁻¹ for 234, 1638 cm⁻¹ for 243, 1642 cm⁻¹ for 201, 1641 cm⁻¹ for 209, 1658 cm⁻¹ for 219, 1654 cm⁻¹ for 231, 1651 cm⁻¹ for 235, 1642 cm⁻¹ for 244.

New bands arising from the —CH₃ group were observed between 2977-2898 cm⁻¹.

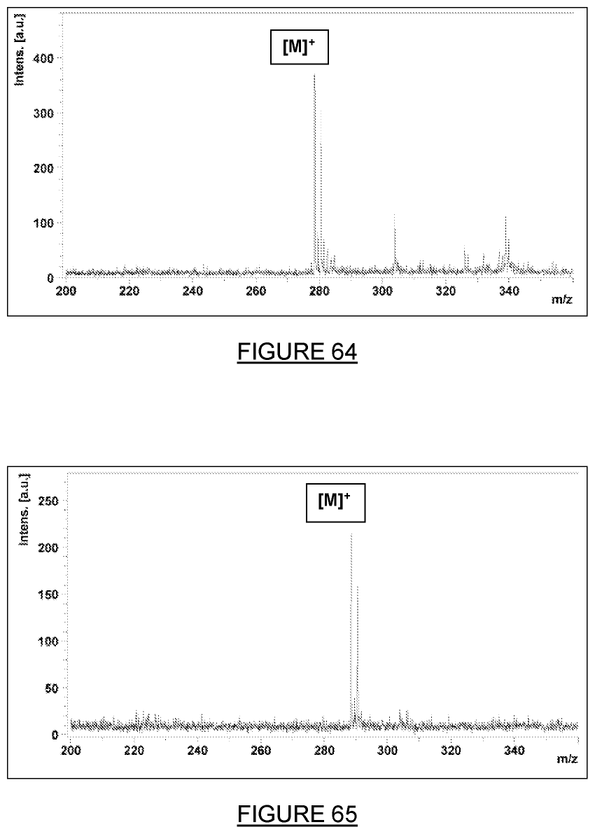

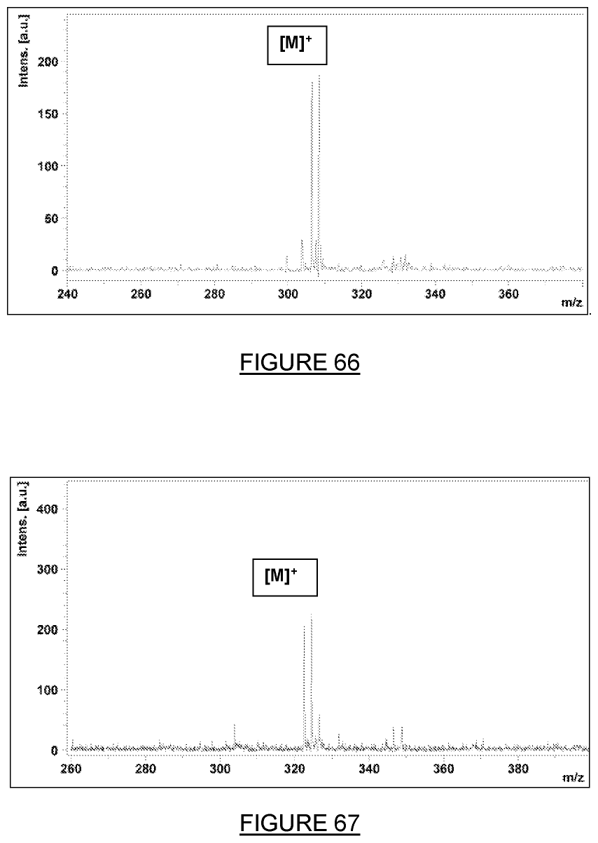

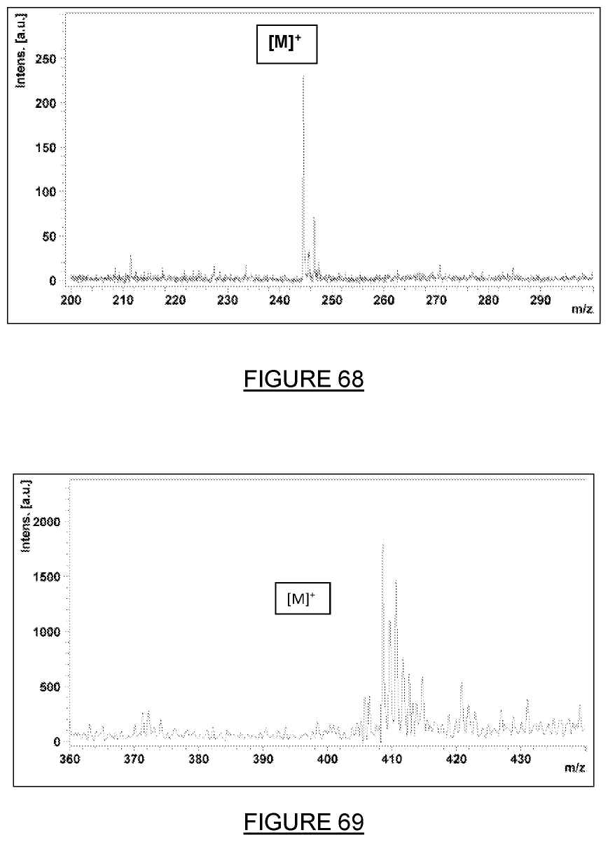

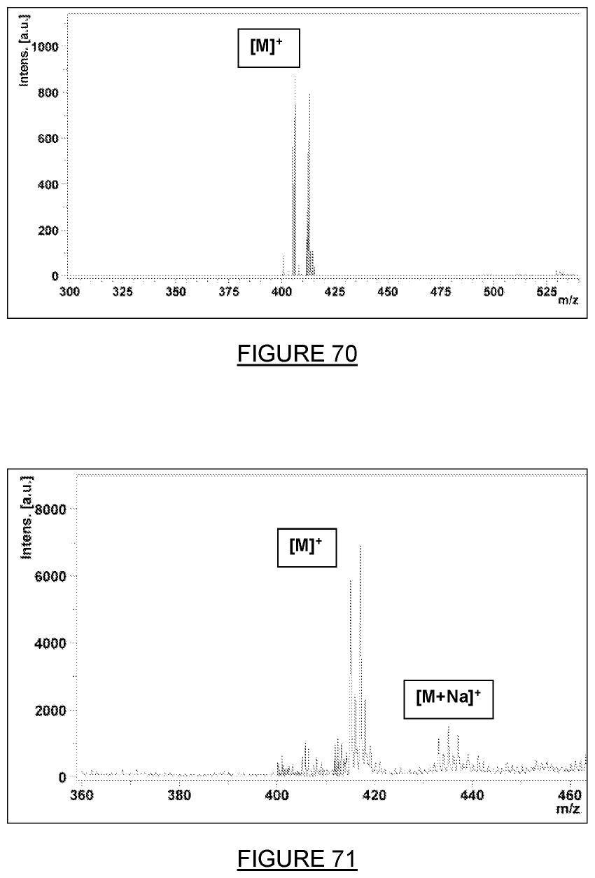

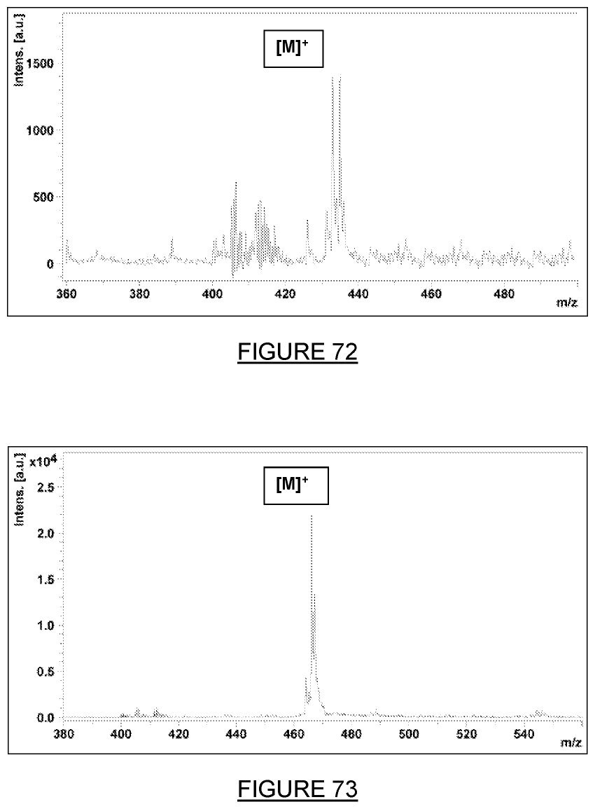

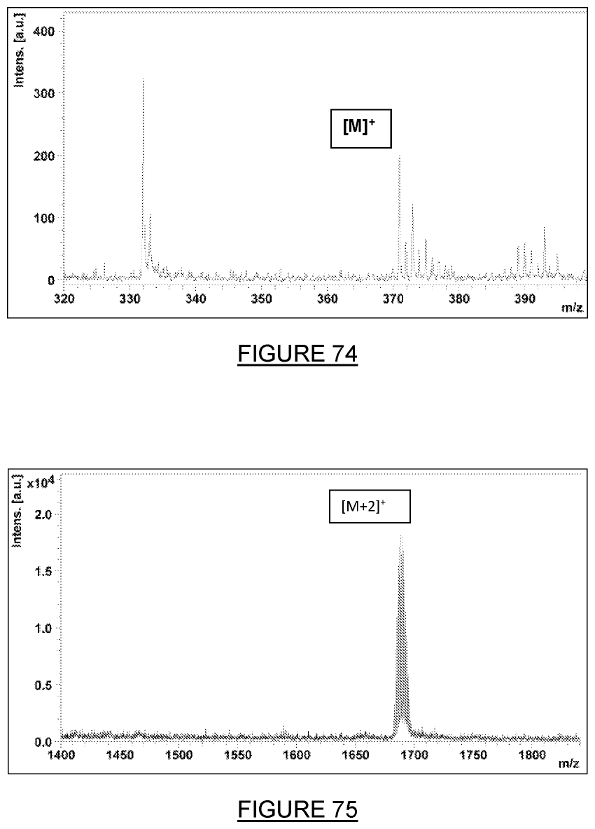

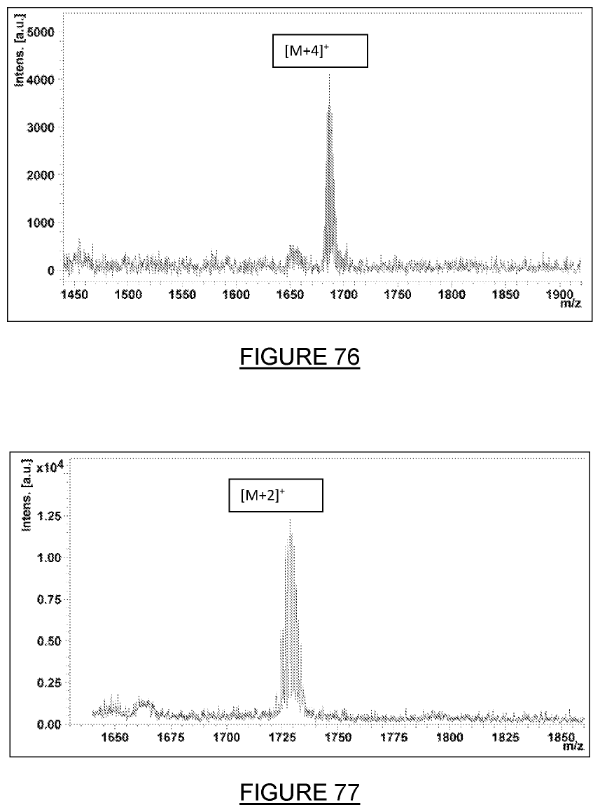

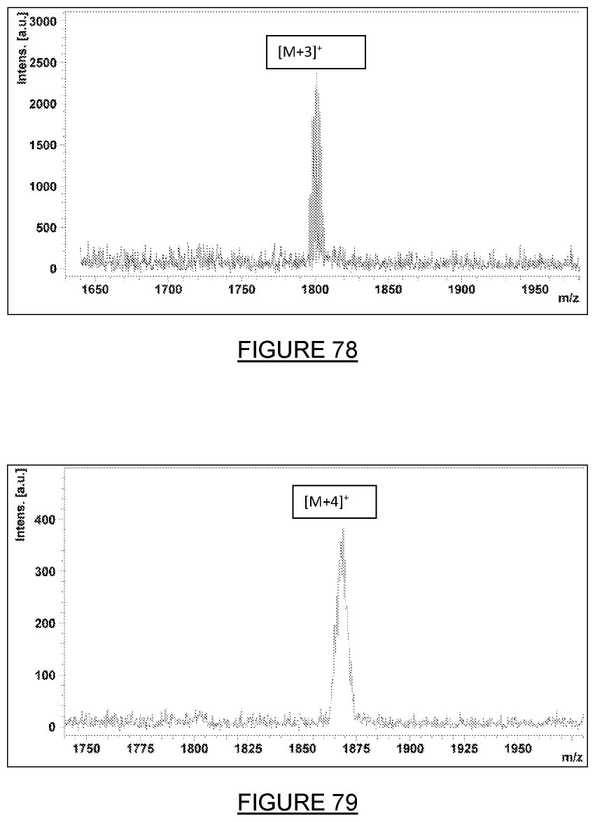

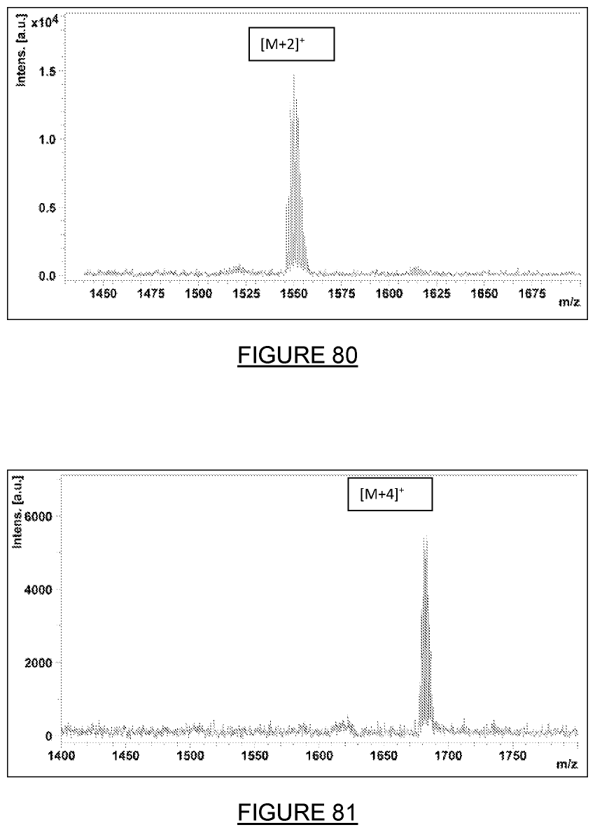

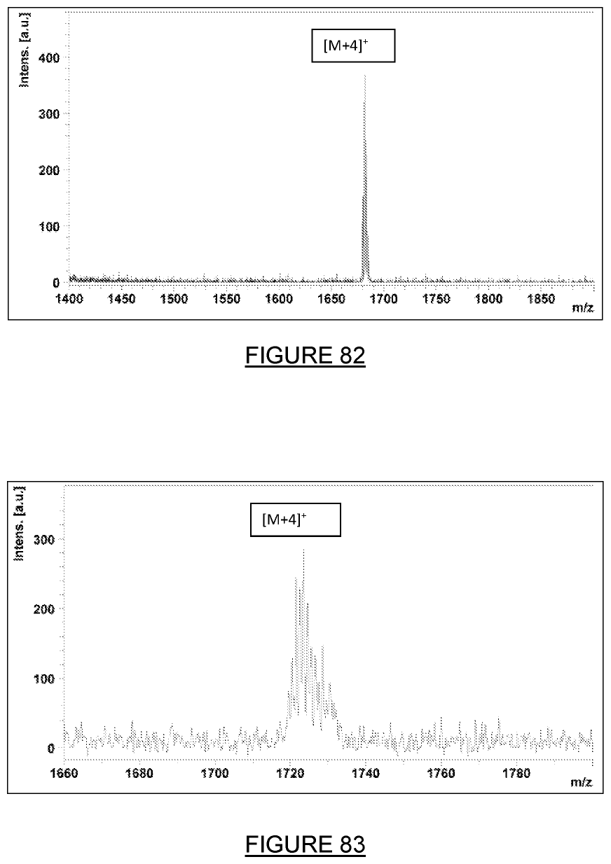

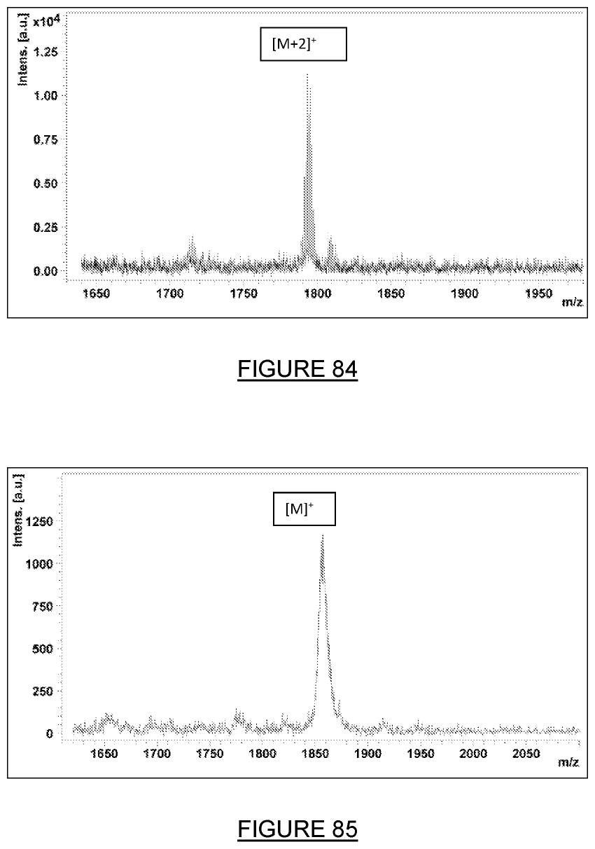

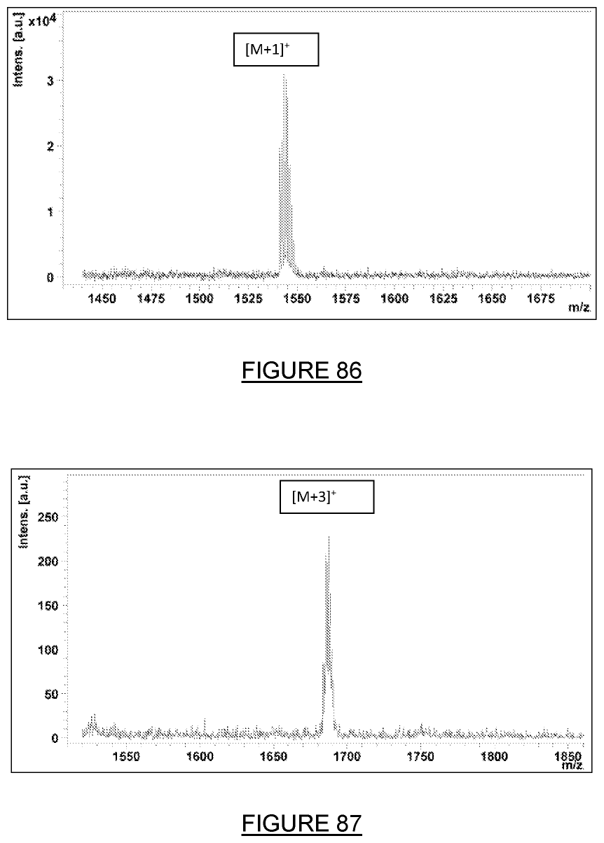

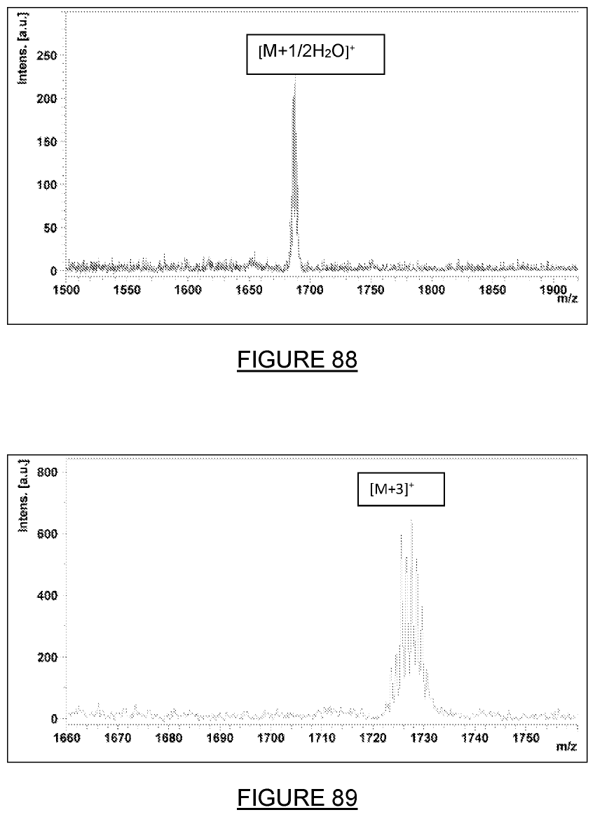

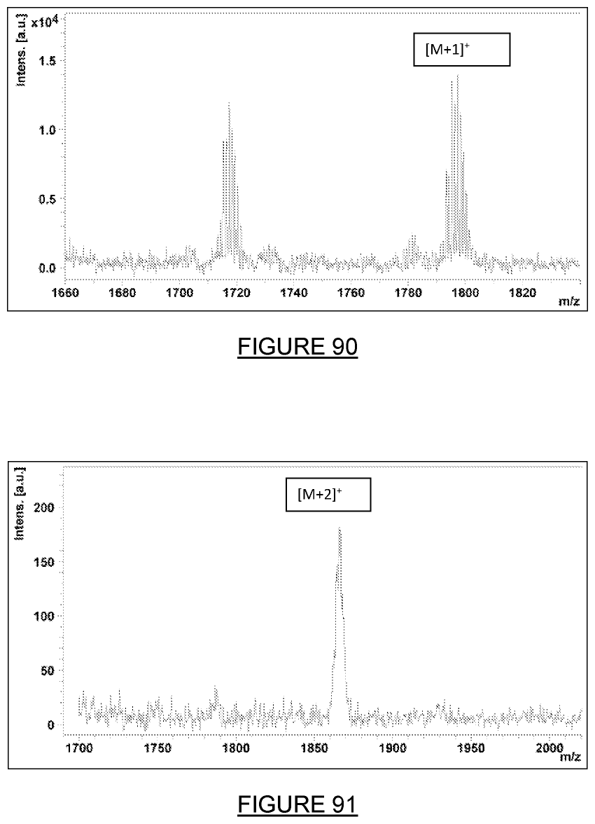

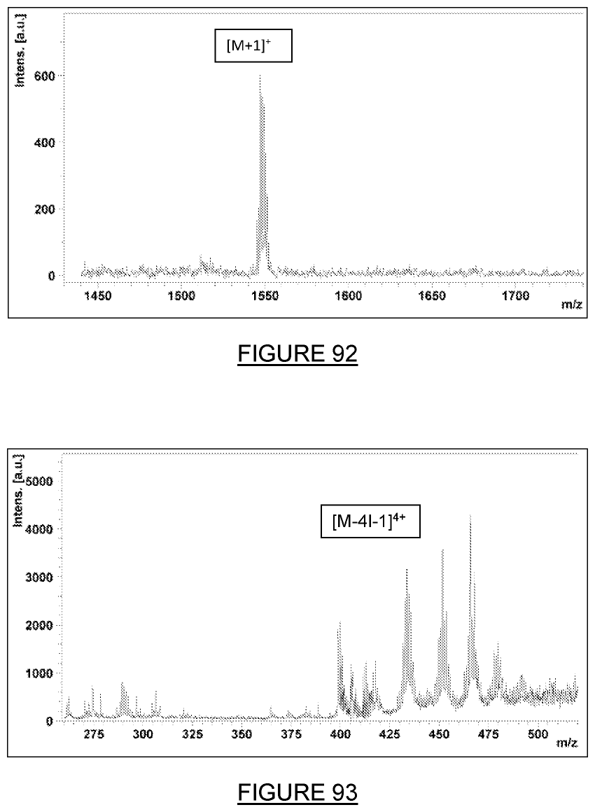

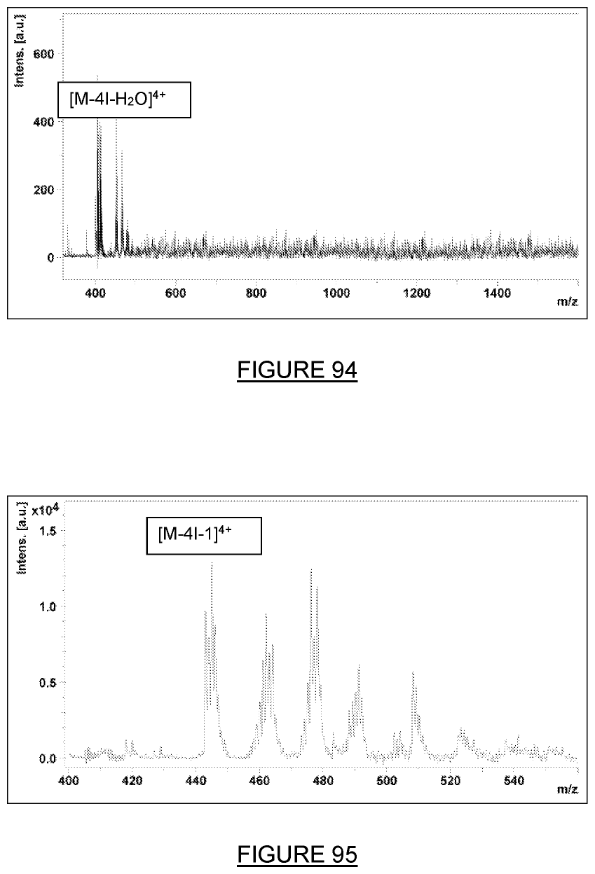

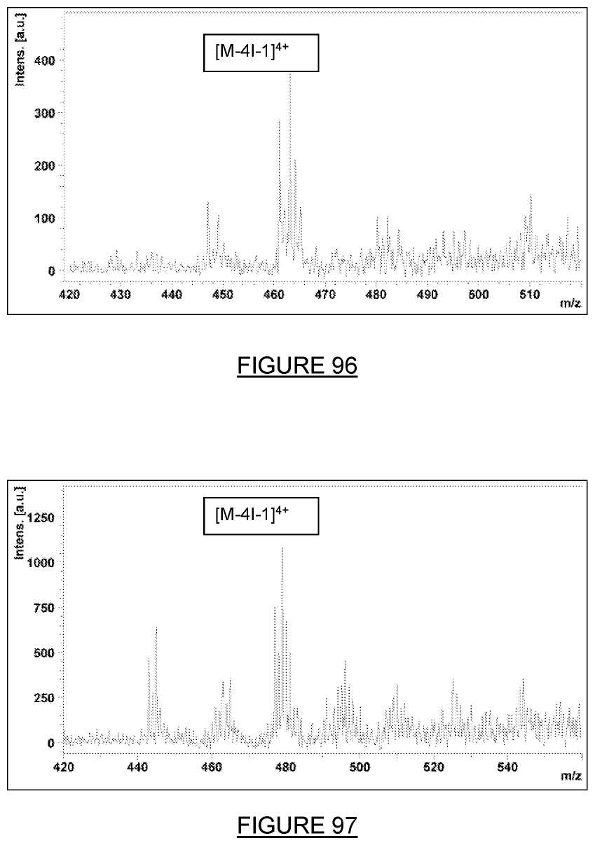

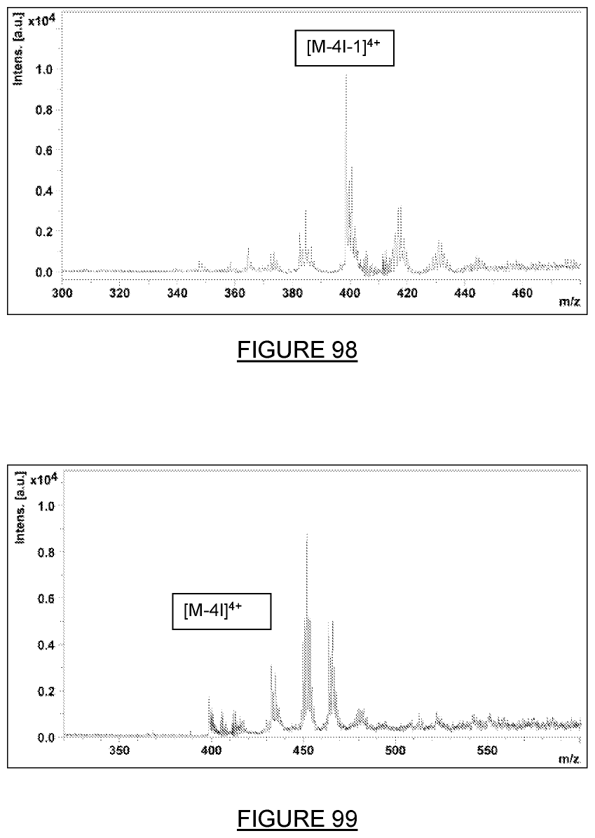

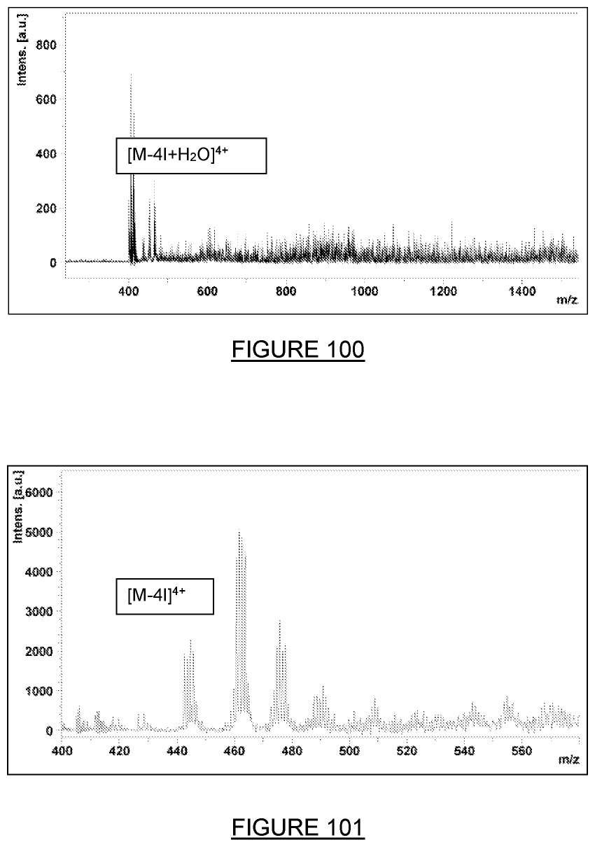

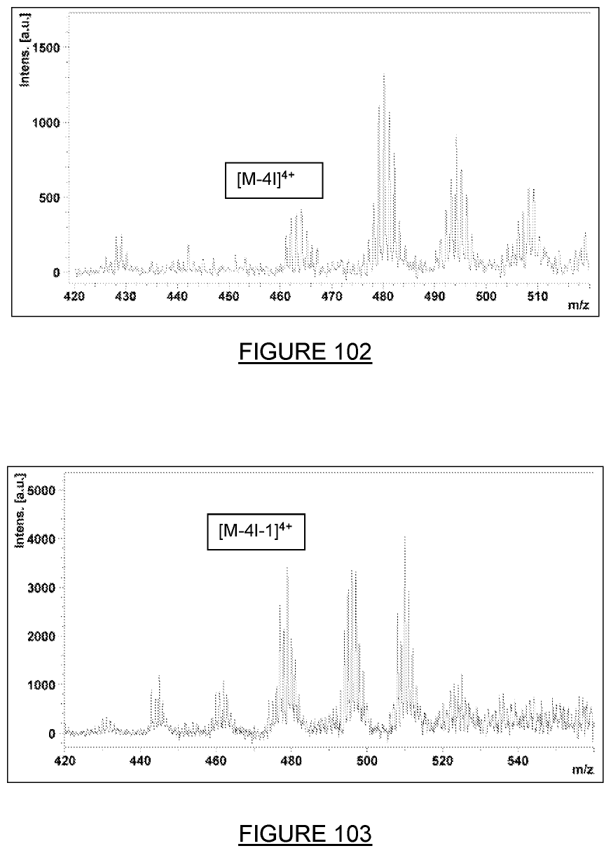

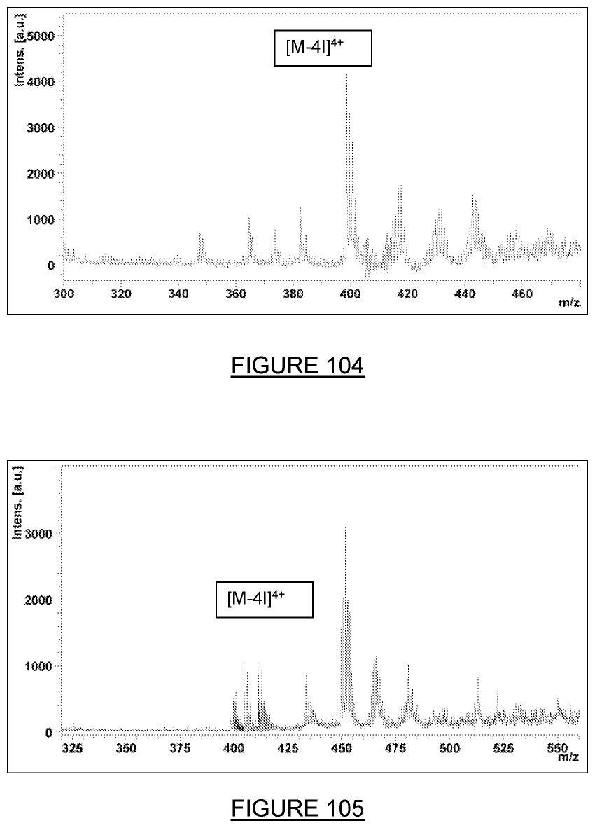

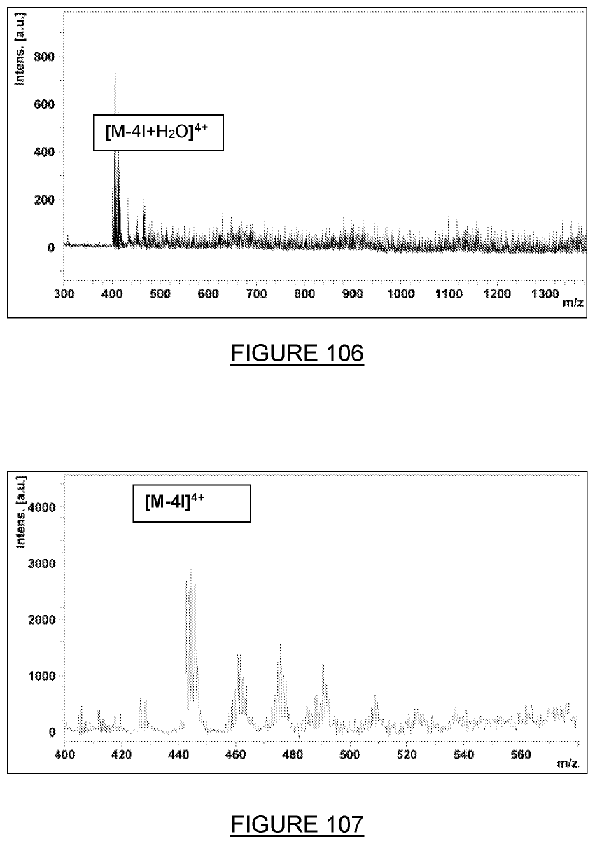

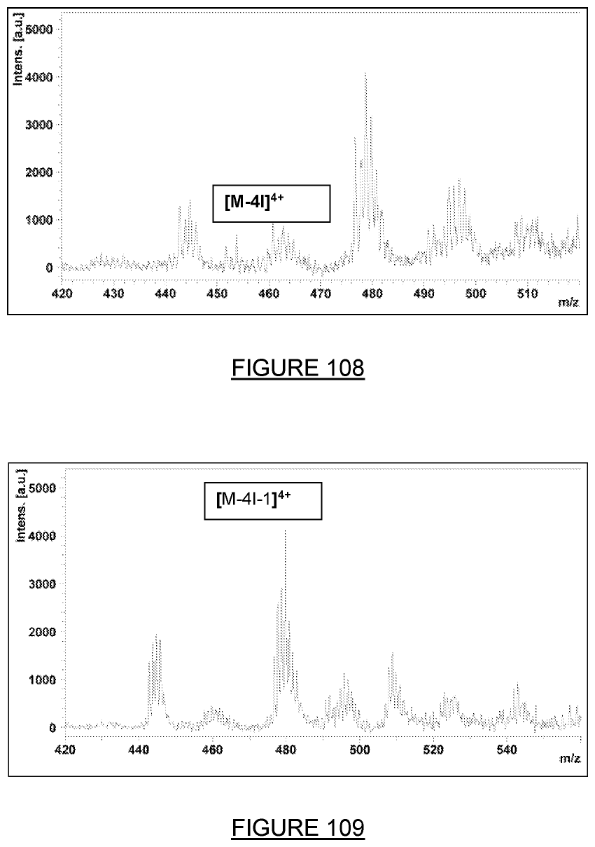

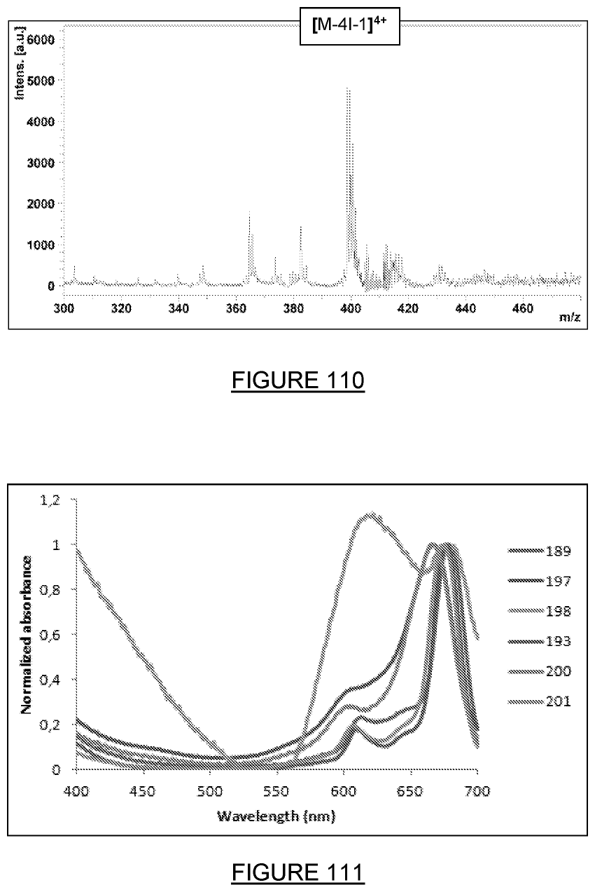

The MALDI-TOF MS data for the compounds are shown in FIG. 63 to FIG. 101.

The MALDI-TOF MS data for all newly synthesized starting compounds agreed with the expected structures. Molecular ion peaks were observed at m/z 279.46 [M]⁺ for 180, m/z 278.56 [M]⁺ for 181, m/z 288.47 [M+H]⁺ for 182, m/z 306.51 [M+H]⁺ for 184, m/z 324.50 [M+H]⁺ for 185, m/z 244.56 [M]⁺ for 215, m/z 405.77 [M]⁺ for 186, m/z 406.41 [M+H]⁺ for 187, m/z 415.04 [M]⁺ for 188, m/z 432.97 [M]⁺ for 190, m/z 467.25 [M+H₂O]⁺ for 191, m/z 370.91 [M]⁺ for 216.

The MALDI-TOF MS data for neutral Pcs (189, 203, 211, 221, 225, 237, 197, 204, 212, 222, 226, 238, 198, 205, 213, 223, 227 and 239) were consistent with the expected structures by showing the molecular ion peaks at m/z 1688.30 [M+2]⁺ for 189, m/z 1690.45 [M+4]⁺ for 203, m/z 1728.30 [M+2]⁺ for 211, m/z 1801.21 [M+3]⁺ for 221, m/z 1868.92 [M+4]⁺ for 225, m/z 1550.23 [M+2]⁺ for 237, m/z 1683.32 [M+4]⁺ for 197, m/z 1681.59 [M+4]⁺ for 204, m/z 1723.55 [M+4]⁺ for 212, m/z 1793.16 [M+2]⁺ for 222, m/z 1856.09 [M]⁺ for 226, m/z 1544.24 [M+1]⁺ for 238, m/z 1687.57 [M+3]⁺ for 198, m/z 1687.93 [M+½H₂O]⁺ for 205, m/z 1727.55 [M+3]⁺ for 213, m/z 1797.31 [M+1]⁺ for 223, m/z 1864.16 [M+2]⁺ for 227, m/z 1547.28 [M+1]⁺ for 239.

After the quaternization reactions to obtain 193, 200, 201, 207, 208, 209, 217, 218, 219, 229, 230, 231, 233, 234, 235, 242, 243 and 244, the charged ion peaks were observed at m/z 435.76 [M−4I−1]⁴⁺ for 193, m/z 454.11 [M−4I−H₂O]⁴⁺ for 207, m/z 445.00 [M−4I−1]⁴⁺ for 217, m/z 463.10 [M−4I−1]⁴⁺ for 229, m/z 479.07 [M−4I−1]⁴⁺ for 233, m/z 400.67 [M−4I−1]⁴⁺ for 242, m/z 435.74 [M−4I]⁴⁺ for 200, m/z 452.13 [M−4I+H₂O]⁴⁺ for 208, m/z 444.62 [M−4I]⁴⁺ for 218, m/z 463.10 [M−4I]⁴⁺ for 230, m/z 478.81 [M−4I−1]⁴⁺ for 234, m/z 400.68 [M−4I]⁴⁺ for 243, m/z 435.65 [M−4I]⁴⁺ for 201, m/z 435.66 [M−4I]⁴⁺ for 209, m/z 445.63 [M−4I]⁴⁺ for 219, m/z 464.76 [M−4I]⁴⁺ for 231, m/z 479.79 [M−4I−1]⁴⁺ for 235, m/z 400.66 [M−4I−1]⁴⁺ for 244.

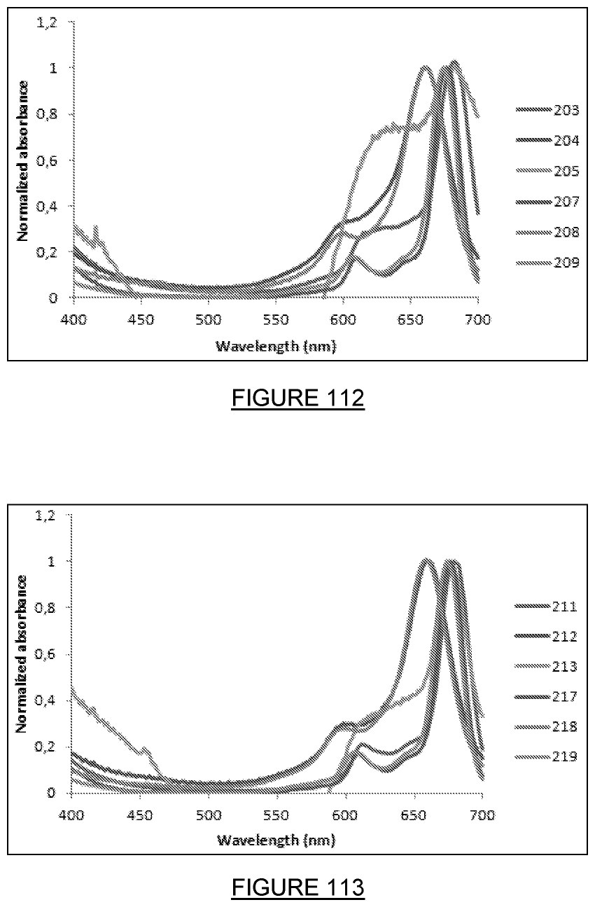

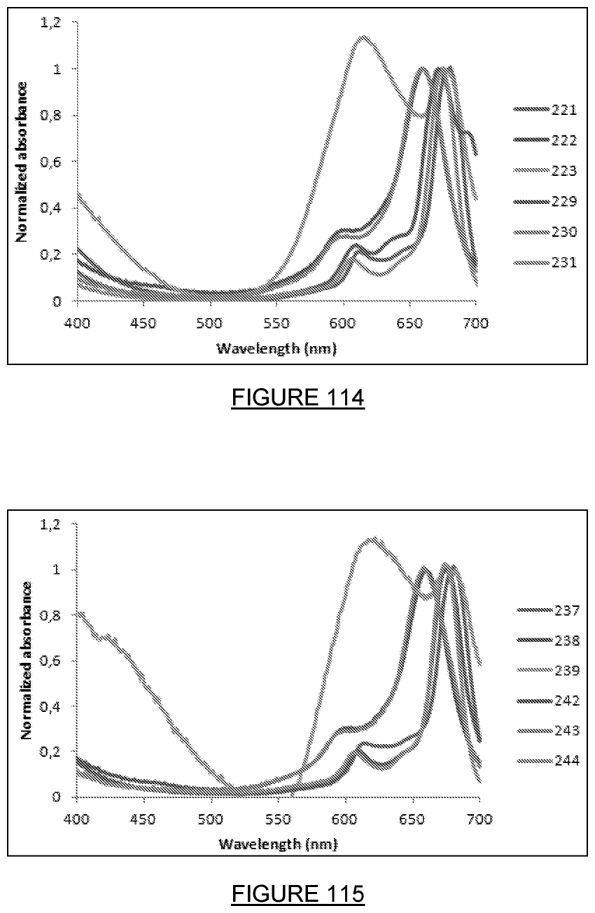

The normalized visible absorption spectra for the compounds are shown in FIG. 111 to FIG. 115.

In the UV-vis spectra of the prepared phthalocyanines, the Q bands, which are one of the characteristic bands for phthalocyanines, were observed in the visible region at 670 nm for 189, 666 nm for 197, 677 nm for 198, 677 nm for 193, 666 nm for 200, 675 nm for 201, 679 nm for 203, 661 nm for 204, 678 nm for 205, 677 nm for 207, 659 nm for 208, 676 nm for 209, 679 nm for 211, 662 nm for 212, 676 nm for 213, 676 nm for 217, 661 nm for 218, 674 nm for 219, 680 nm for 221, 662 nm for 222, 677 nm for 223, 672 nm for 229, 659 nm for 230, 675 nm for 231, 680 nm for 221, 662 nm for 222, 677 nm for 223, 672 nm for 229, 659 nm for 230, 675 nm for 231, 679 nm for 237, 665 nm for 238, 679 nm for 239, 675 nm for 242, 659 nm for 243, 677 nm for 244.

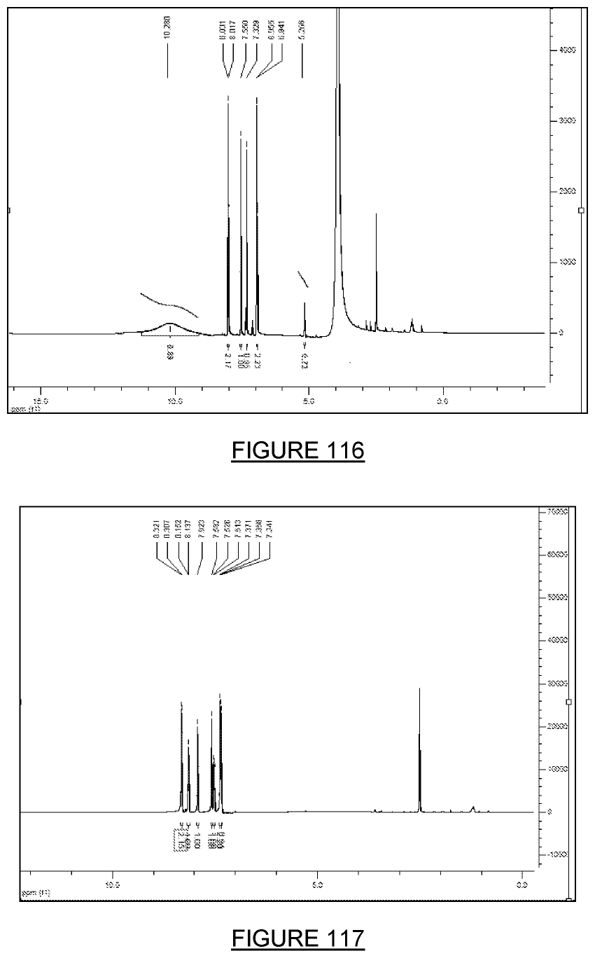

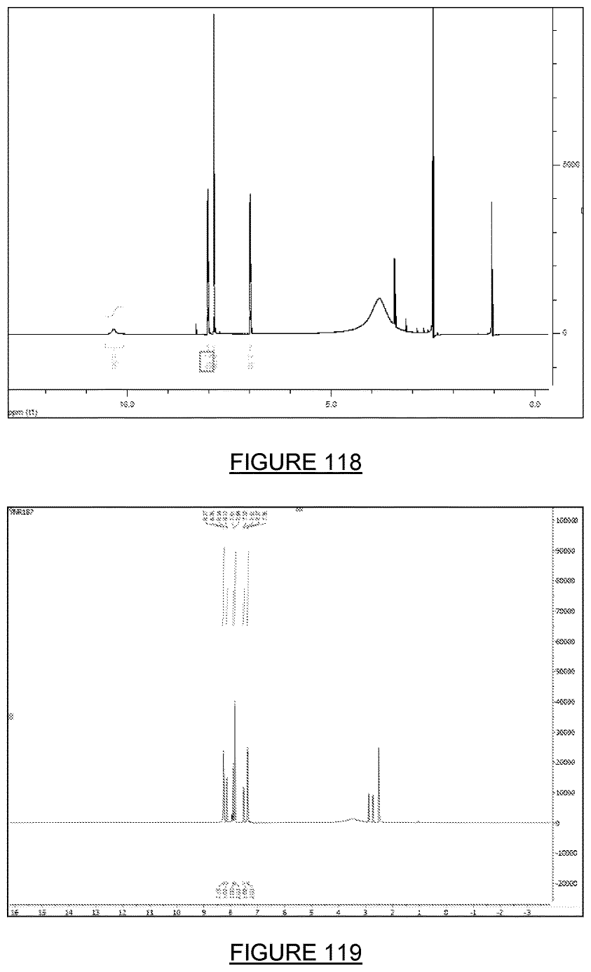

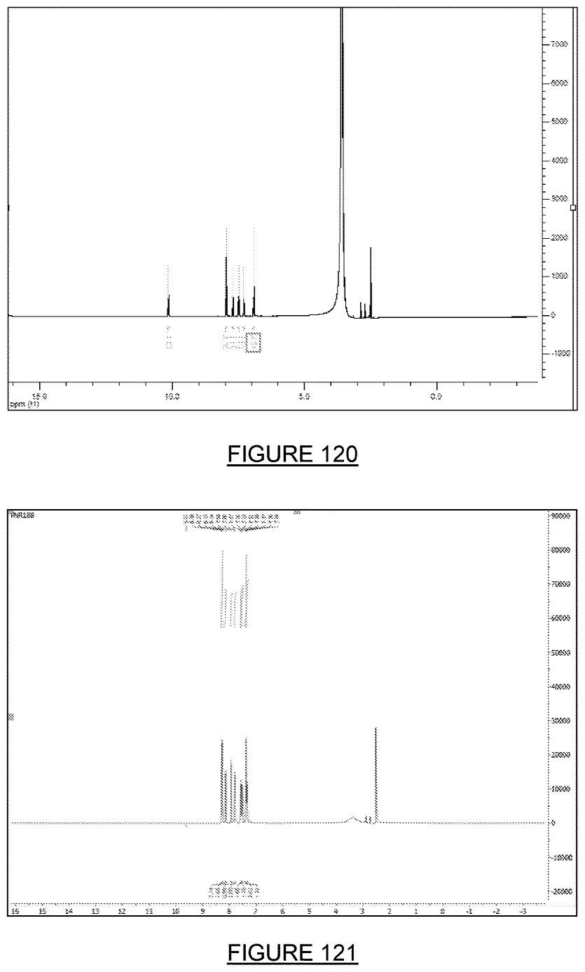

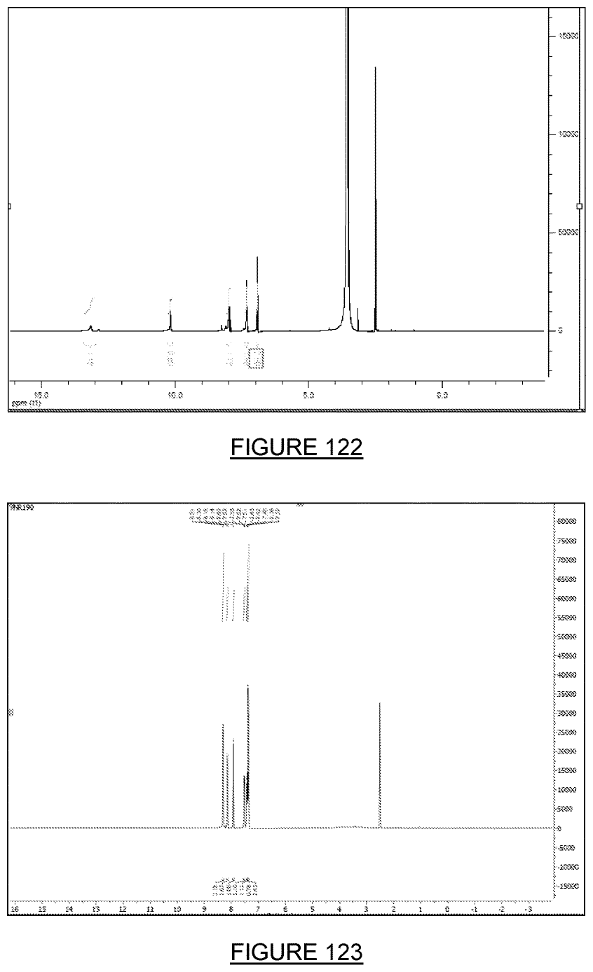

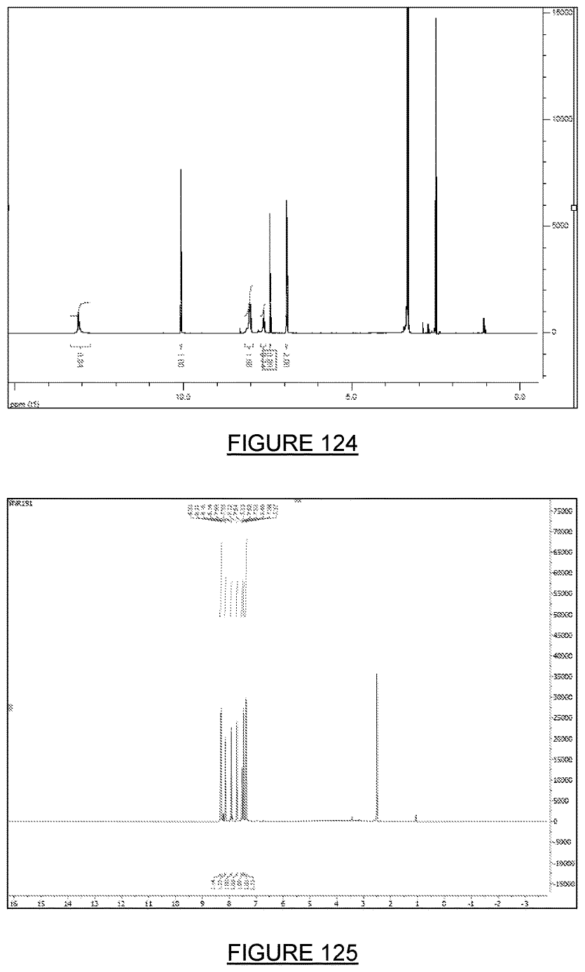

The ¹H-NMR and ¹³C-NMR data for the compounds are shown in FIG. 116 to FIG. 125.

The ¹H-NMR and ¹³C-NMR data presented consistent results for studied structures. When compared the ¹H-NMR spectra of compound 180 and 186, 181 and 187, 182 and 188, 184 and 190, 185 and 191, 215 and 216, the absence of —OH proton signal of 4-(4,6-dichloro-1H-benzo[d]imidazol-2-yl)phenol (180), 4-(5,6-dichloro-1H-benzo[d]imidazol-2-yl)phenol (181), 4-(5-bromo-1H-benzo[d]imidazol-2-yl)phenol (182), 4-(4-bromo-6-fluoro-1H-benzo[d]imidazol-2-yl)phenol (184), 4-(6-bromo-4-chloro-1H-benzo[d]imidazol-2-yl)phenol (185), 4-(5-chloro-1H-benzo[d]imidazol-2-yl)phenol (215) and the arising the new peaks in aromatic region at 8.31-7.34 ppm for 186, 8.26-7.37 ppm for 187, 8.27-7.35 ppm for 188, 8.30-7.37 ppm for 190, 8.31-7.37 ppm for 191, 8.27-7.23 ppm for 216 were the proof that the nitro displacement reaction was occurred to give phthalonitrile derivatives.

In the ¹³C-NMR spectrum of the phthalonitrile derivatives, presence of the signals at 116.34 ppm, 115.84 ppm for 186, 116.34 ppm, 115.83 ppm for 187, 116.37 ppm, 115.85 ppm for 188, 116.35 ppm, 115.84 ppm for 190, 116.34 ppm, 115.84 ppm for 191, 116.31, 115.80 for 216 based on the nitrile carbon atoms are clearly different from their starting compounds.

1.-22. (canceled)

23. A compound of general Formula (I) 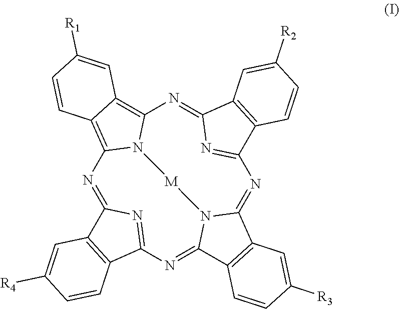
wherein
M is metal selected from the group consisting of zine, indium, gallium cobalt, copper and nickel;
R₁-R₄ is selected from the group consisting of 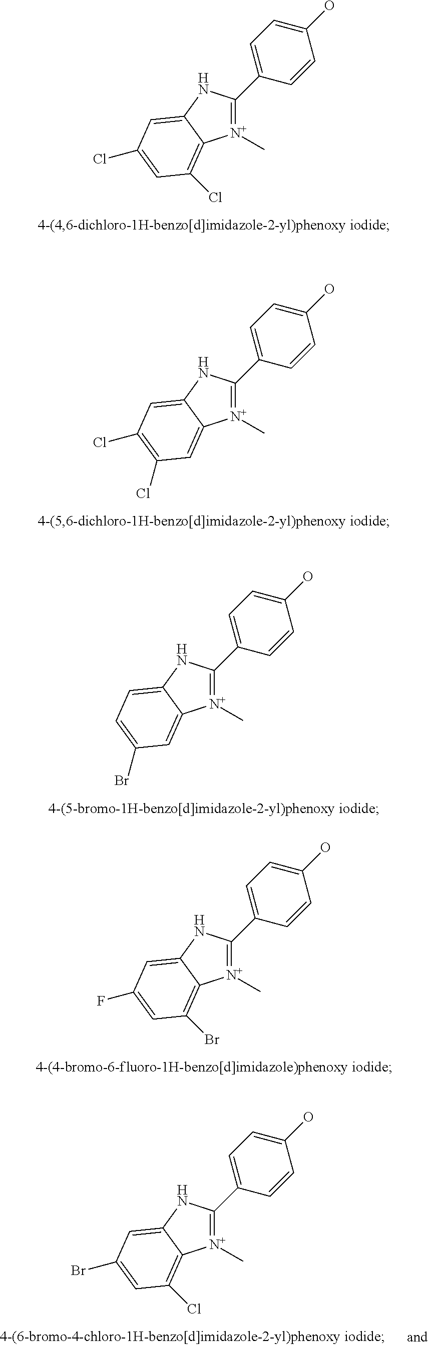 [SMILES: C[n+]1c(-c2ccc(O)cc2)[nH]c2cc(Cl)cc(Cl)c21 ; C[n+]1c(-c2ccc(O)cc2)[nH]c2cc(Cl)c(Cl)cc21 ; C[n+]1c(-c2ccc(O)cc2)[nH]c2ccc(Br)cc21 ; C[n+]1c(-c2ccc(O)cc2)[nH]c2cc(F)cc(Br)c21 ; C[n+]1c(-c2ccc(O)cc2)[nH]c2cc(Br)cc(Cl)c21] 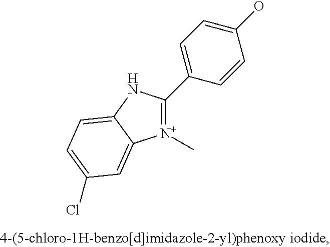 [SMILES: C[n+]1c(-c2ccc(O)cc2)[nH]c2ccc(Cl)cc21]
and combinations thereof.

24. The compound of claim 23, wherein the compound is selected from:

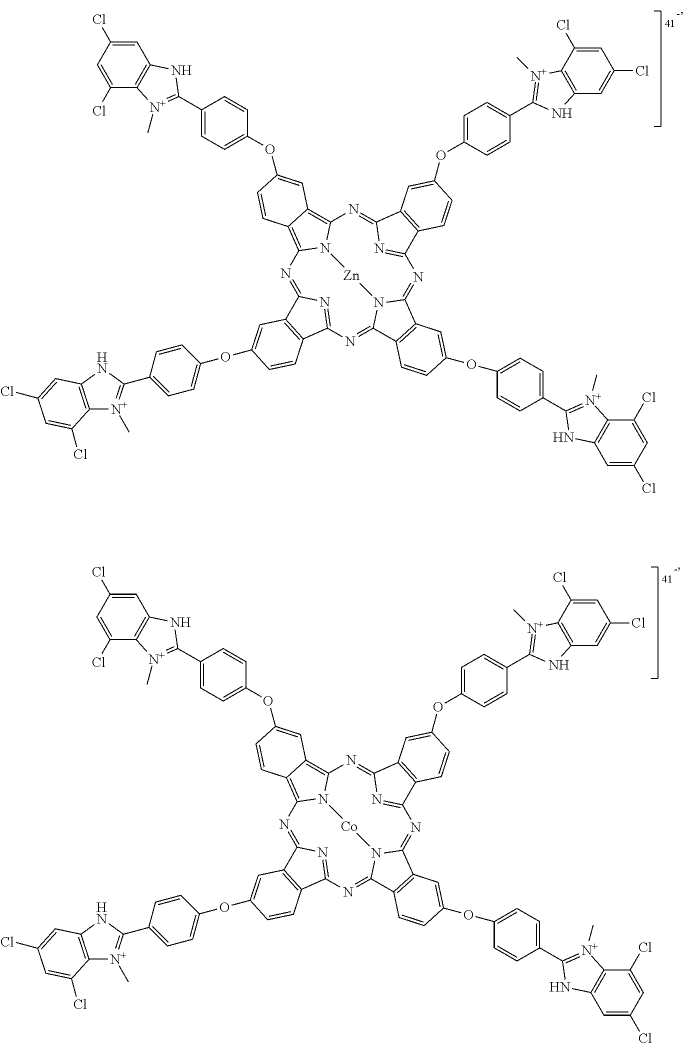

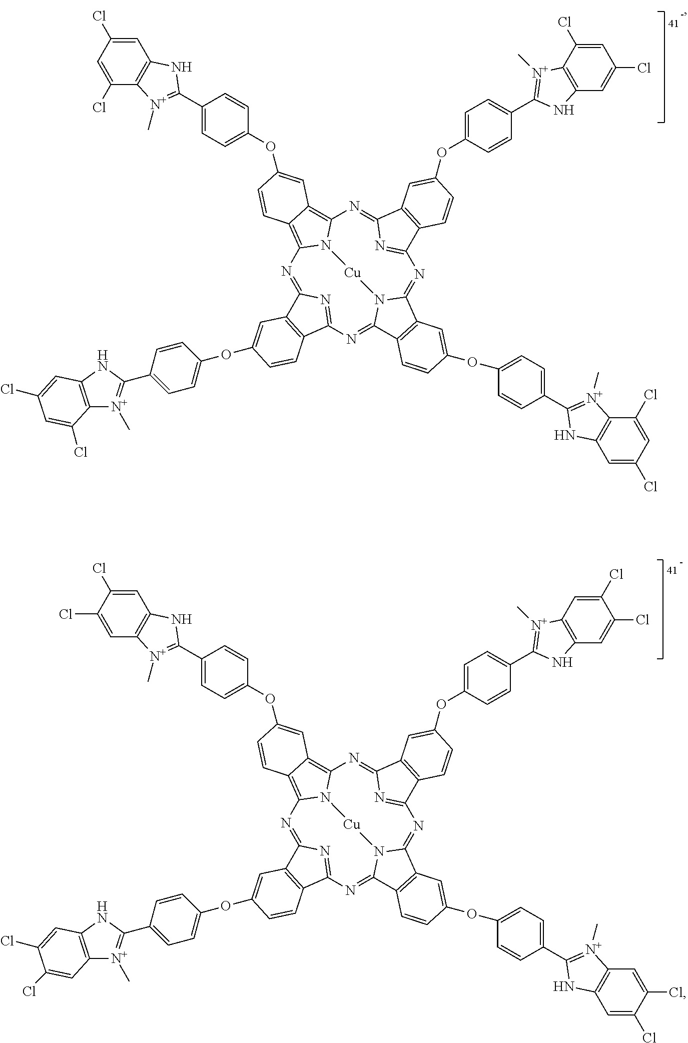

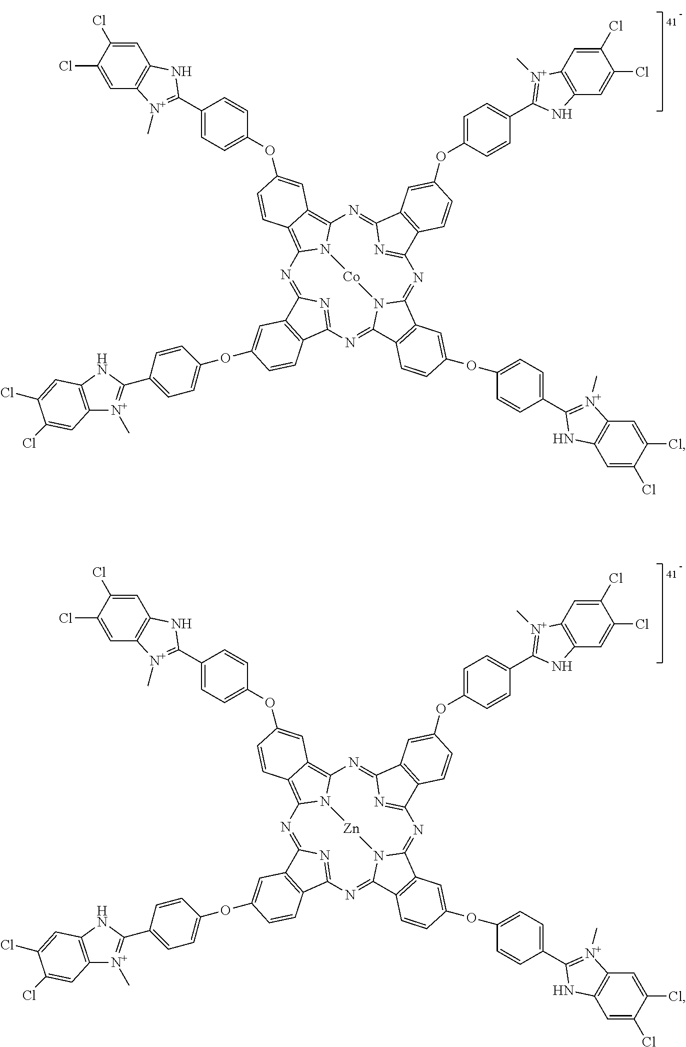

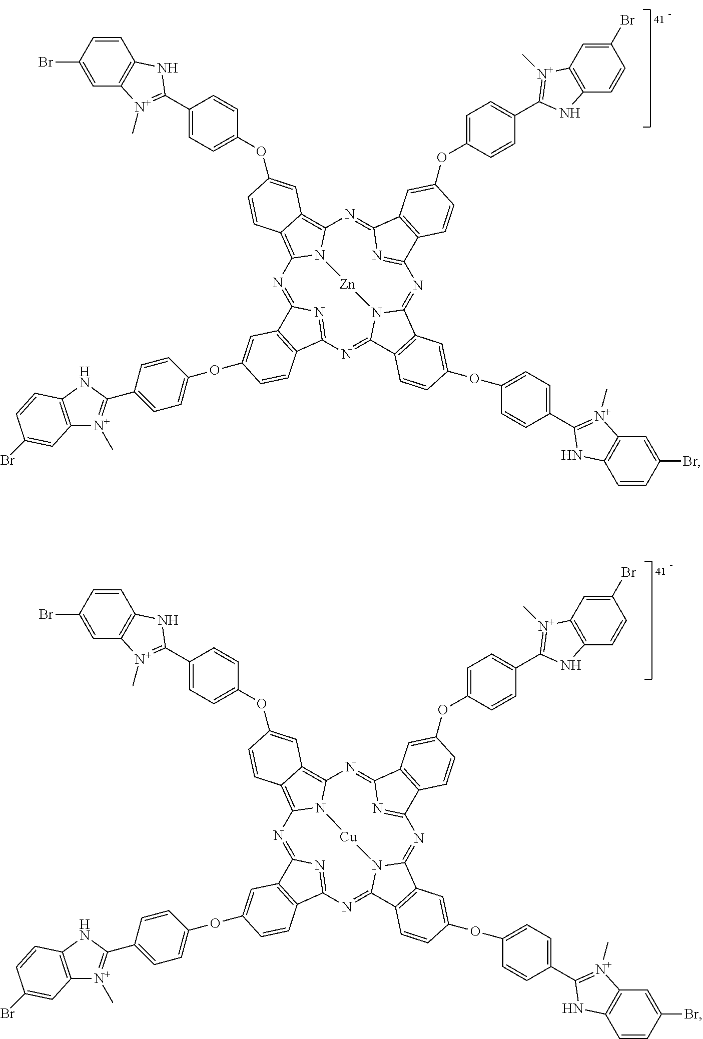

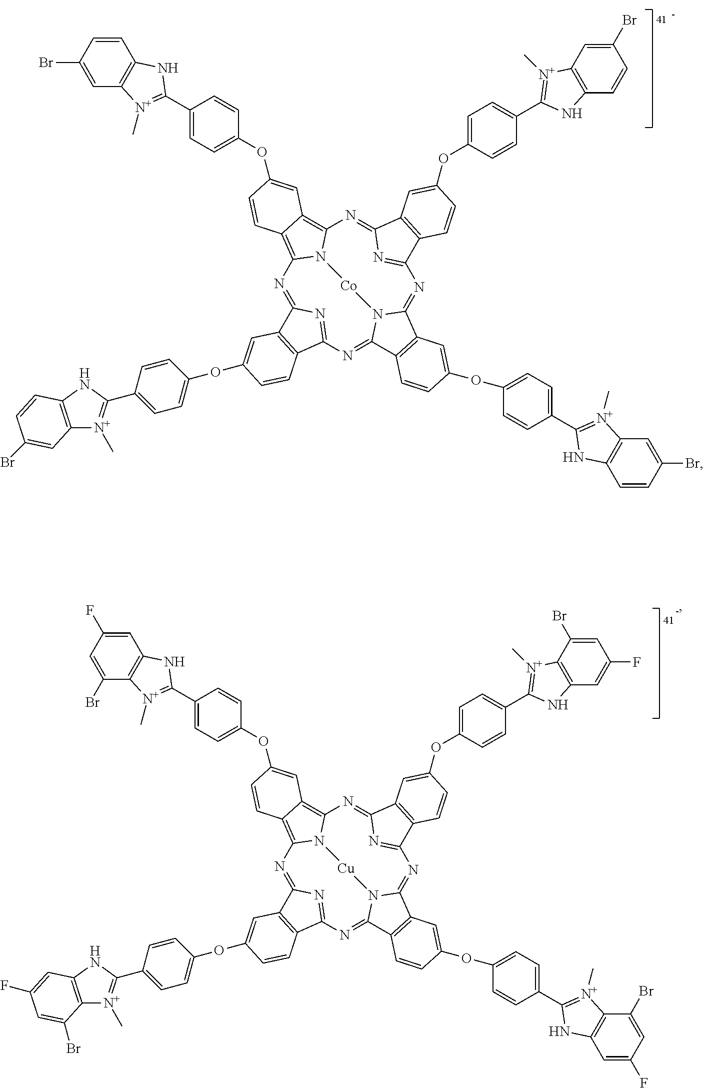

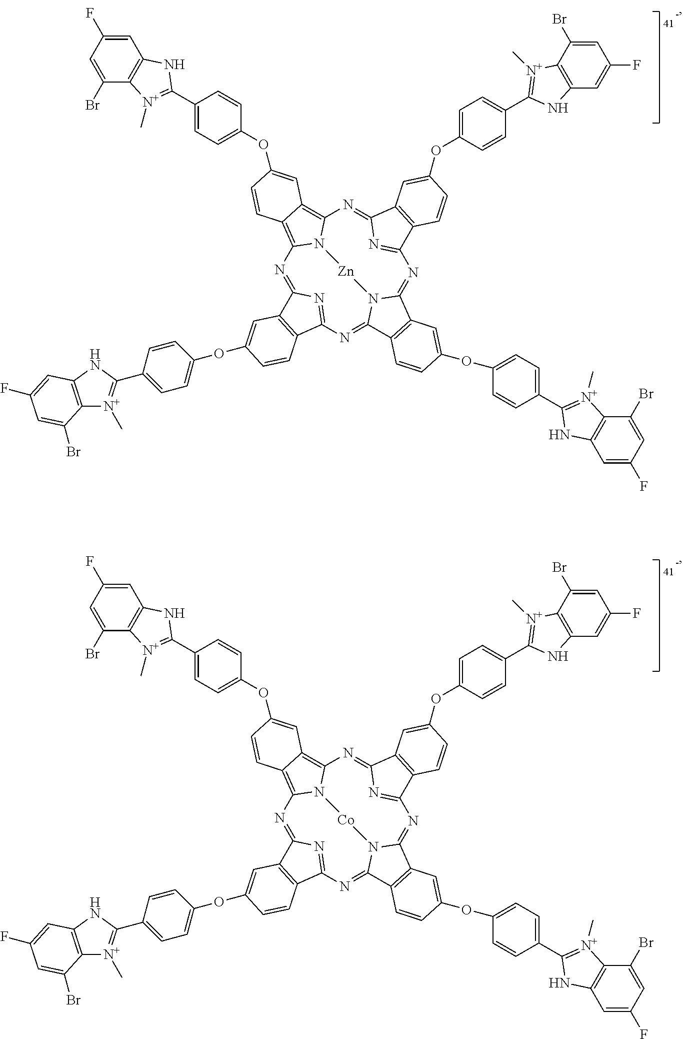

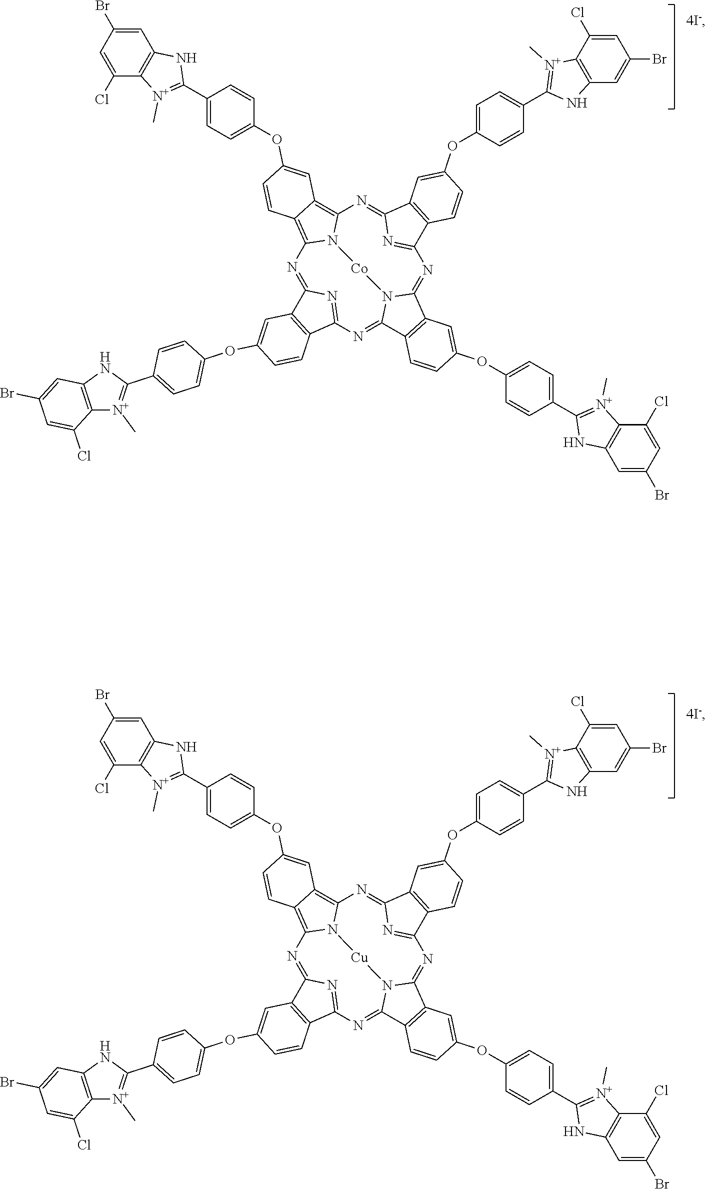

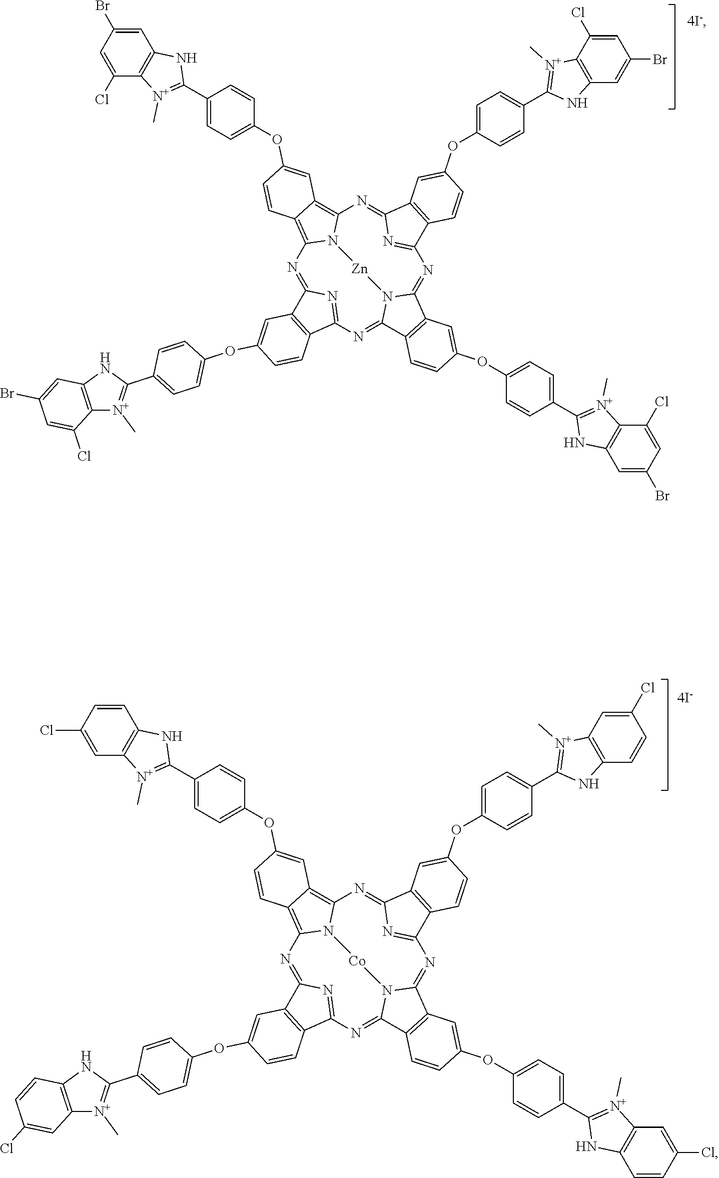

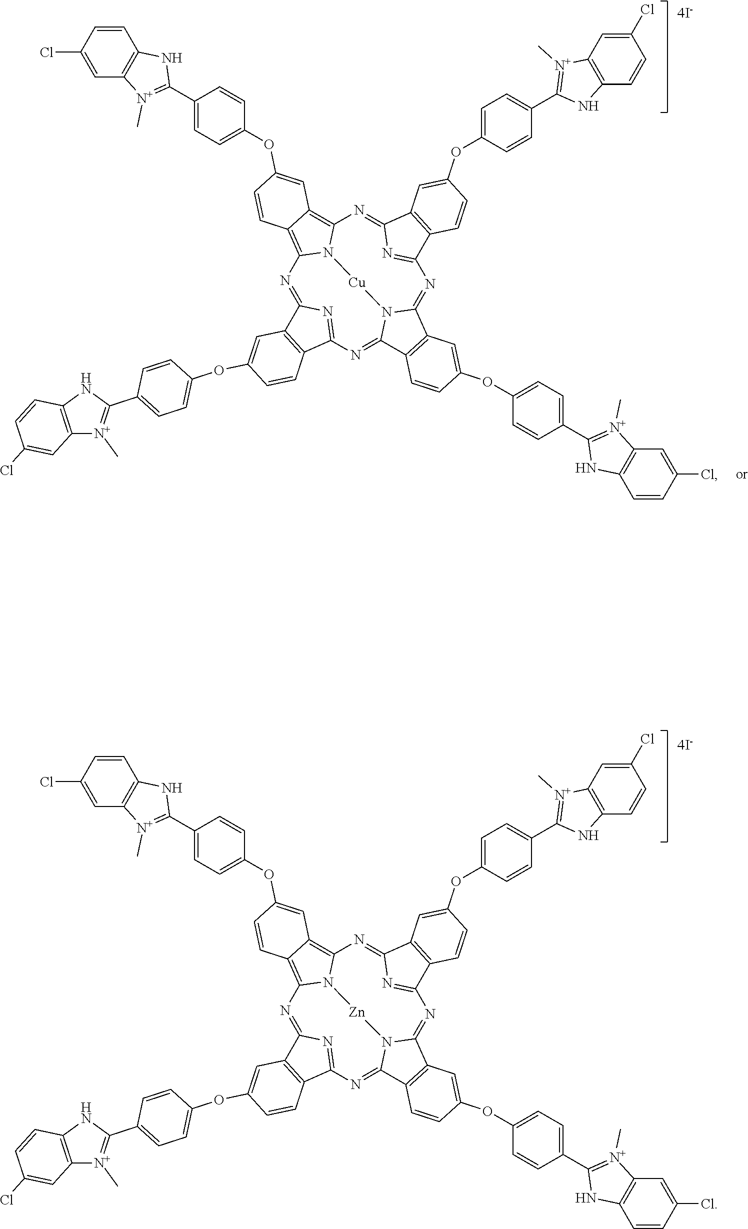

25. A method of treatment of microbial infections, comprising administering a therapeutically effective amount of the compound of claim 23 to a subject in need thereof.

26. The method of claim 25, wherein the microbial infections are Escherichia coli (E. coli) infections.

27. The method of claim 25, wherein the microbial infections are Staphylococcus aureus (S. aureus) infections.

28. The method of claim 25, wherein the microbial infections are protozoan infections.

29. A method of treatment of microbial infections, comprising administering a therapeutically effective amount of the compound of claim 24 to a subject in need thereof.

30. The method of claim 29, wherein the microbial infections are Escherichia coli (E. coli) infections.

31. The method of claim 29, wherein the microbial infections are Staphylococcus aureus (S. aureus) infections.

32. The method of claim 29, wherein the microbial infections are protozoan infections.
